(19) United States
(12) Patent Application Publication
(10) Pub. No.: US 2014/0045826 A1
(43) Pub. Date: Feb. 13, 2014

(54) METHODS AND COMPOSITIONS FOR TREATING NEURODEGENERATIVE DISEASES

(71) Applicants: William C. Shakespeare, Southborough, MA (US); Frank G. Haluska, Wayland, MA (US)

(72) Inventors: William C. Shakespeare, Southborough, MA (US); Frank G. Haluska, Wayland, MA (US)

(73) Assignee: ARIAD PHARMACEUTICALS, INC., Cambridge, MA (US)

(21) Appl. No.: 14/009,565

(22) PCT Filed: Apr. 6, 2012
(86) PCT No.: PCT/US12/32566

§ 371 Date: Oct. 3, 2013

Related U.S. Application Data
(60) Provisional application No. 61/472,961, filed on Apr. 7, 2011.
(60) Provisional application No. 61/518,427, filed on May 5, 2011.

(51) Int. Cl. C07D 487/04; C07D 471/04

(52) U.S. Cl. CPC C07D 487/04; C07D 471/04
USPC 514/218; 514/249; 514/300; 514/253.04; 514/248; 514/245; 514/233.2

(57) ABSTRACT

The invention discloses methods and compositions for treating or preventing neurodegenerative disease by administering a compound of Formula I: or a pharmaceutically acceptable salt, solvate or hydrate thereof, wherein the variables are defined as herein.

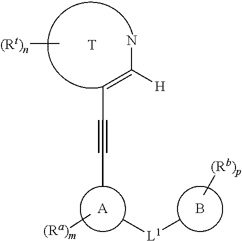

### RELATED APPLICATION DATA

This application claims the benefit of U.S. Provisional Application Nos. 61/472,961 filed Apr. 7, 2011 and 61/518,427 filed May 5, 2011, both of which are hereby incorporated herein by reference in their entirety.

### TECHNICAL FIELD

This invention relates to methods for treating or preventing neurodegenerative diseases by administering a targeted tyrosine kinase inhibitor disclosed herein or a pharmaceutically acceptable salt thereof.

### BACKGROUND

While many details concerning the biological mechanisms underlying the development and progression of neurodegenerative diseases remain unclear, there have been important recent developments. For instance, both Parkinson's disease (PD) and Alzheimer's disease (AD), have been linked to protein misfolding and aggregation (see Gregersen N. J Inherit Metab Dis 29:456 (2006), and Whatley et al., Biochim Biophys Acta 1782:700 (2008)). The accumulation of misfolded proteins in these diseases appears to arise from an imbalance in the generation and clearance of misfolded proteins. Protein misfolding can occur as a result of genetic mutations, environmental insults or oxidative damage (Olzmann et al., Curr Med Chem 15:47 (2008)). As for clearance of such misfolded proteins, the ubiquitin-proteasome system and the aggresome-autophagy pathway both appear to be important cellular defense mechanisms against toxic build-up of misfolded proteins (Kopito RR. Trends Cell Biol 10:524-530 (2000); Xie et al., Nat Cell Biol 9:1102-1109 (2007); and Levine et al., Cell 2008, 132:27-42 (2008)). The failure of cells to cope with excess misfolded proteins is believed to be a common pathological mechanism linking these clinically distinct diseases.

Recent studies suggest that the protein, parkin, may play an important role. Parkin-mediated Lys63-linked polyubiquitination of misfolded proteins promotes their sequestration into aggresomes and subsequent clearance by autophagy (Olzmann et al., J Cell Biol 178:1025 (2007); and Olzmann et al., Autophagy 4:85 (2008)). Furthermore, loss-of-function mutations in parkin are a major cause of recessively transmitted neurodegenerative diseases, such as early-onset PD (Kitada et al., Nature 392:605 (1998); and Hattori et al., Lancet 364:722 (2004)). Parkin has also been reported to play a role in targeting damaged mitochondria for mitophagy (Narendra et al., J Cell Biol 183:795 (2008)), and so may be implicated in neurodegenerative diseases associated with mitochondrial dysfunction. Parkin has also been reported to protect dopamine neurons from tau-induced degeneration (Klein et al., Neurosci Lett. 401:130 (2006)).

Parkin is believed be a pan-neuroprotective agent against a number of different toxic insults including elevated expression of substrates for parkin ubiquitination (Lo Bianco et al., Proc. Natl. Acad. Sci. U.S.A 101:17510 (2004); Petrucelli et al., Neuron 36:1007 (2002); Yamada et al., Hum. Gene Ther 16:262 (2005); and Yang et al., Neuron 37:911 (2003)) as well as other toxins (Darios et al., Hum. Mol. Genet. 12:517 (2003); Hyun et al., J. Neurosci. Res 82:232 (2005); Manfredsson et al., Mol. Therapy. 11(Suppl. 1):24 (2005); and Staropoli et al., Neuron 37:735 (2003)). Increasing parkin expression reduces oxidative damage (Hyun et al., J. Biol. Chem. 277:28572 (2002)) while blocking parkin expression increases oxidative damage (Greene et al., Hum. Mol. Genet. 14:799 (2005); and Palacino et al., J. Biol. Chem. 279:18614 (2004)), which explains the general protection from parkin against a variety of insults.

PD is a chronic, progressive motor system disorder. Approximately 50,000 Americans are diagnosed with PD each year. The primary symptoms of this neurodegenerative disease are trembling, rigidity, slowness of movement, and impaired balance. In addition, many PD patients experience a variety of other symptoms, including emotional changes, memory loss, speech problems, or difficulty sleeping. As the disease progresses, many patients find it increasingly difficult to walk, talk, swallow or carry out simple tasks.

PD is caused by specific and progressive neuronal loss of mid-brain dopamine (DA) neurons. Ordinarily, these neurons produce dopamine, a chemical messenger responsible for transmitting signals between the substantia nigra and the corpus striatum, resulting in smooth, purposeful muscle activity. However, loss of dopamine causes the nerve cells of the striatum to fire in an uncontrolled manner, leaving patients with impaired ability to direct and control their movements, an impairment that can be severe and profoundly crippling.

There is no cure for PD. Current therapy relies heavily on replenishing dopamine by giving patients oral doses of a dopaminergic agent like the dopamine precursor levodopa (alone or in the combination carbidopa/levodopa) or a dopamine agonist. Such therapy can provide relief, although with the increasing risk of serious side effects and often with diminishing therapeutic results, requiring increasing doses as treatment continues, and more serious side effects. There is a profound need for additional therapeutics for PD.

c-Abl is a major regulator of parkin function and phosphorylates parkin on tyrosine 143. This phosphorylation inhibits parkin's E3 ubiquitin ligase activity leading to accumulation of AIMP2 and FBP1 and loss of parkin's cytoprotective function and cell death. One Abl inhibitor, STI-571, has been found to maintain parkin in a catalytically active and neuroprotective state by preventing phosphorylation of parkin. As such, it is believed that inhibition of c-Abl presents a viable approach for the treatment of PD. Ko, et al., PNAS, 107(38), 16691-16696 (2010). One challenge of using STI-571 to treat PD is that it has poor penetration of the blood-brain barrier as demonstrated in mice and humans. Thus, there is a need for Abl inhibitors that cross the blood-brain barrier for the treatment of PD.

Desirable therapies for Alzheimer's disease share certain characteristics with those for PD. Yet, the etiologies of PD and AD are not identical. In the case of AD, therapies are generally directed to the reduction of amyloid-β peptides. Amyloid-β peptides are metabolites of the amyloid precursor protein and are believed to be its major pathological determinant. The proteolytic cleavages that form the amyloid-β N and C termini are catalyzed by β-secretase and γ-secretase, respectively. It is postulated that reducing amyloid-β without affecting Notch-1 cleavage may prove useful as a basis for developing therapies for AD. Netzer, et al., PNAS, 100(21): 12444-12449 (2003). That is, γ-secretase inhibitors that can reduce amyloid-β formation without impairing cleavage of other γ-secretase substrates such as Notch are potentially useful for the treatment of AD. γ-secretase activating protein (GSAP) is a recently identified target that does not interact with Notch and its reduced concentrations in cell lines was associated with decreased amyloid-β concentrations. He, et al., Nature., 467: 95-98 (2010). Compounds that accumulate in the brain and target GSAP represent a valid approach for development of AD therapies. Id.

In the context of testing STI-571 in their models, Netzer, et al hypothesized that Abl kinase is not believed to be required for amyloid-β production, although it may tangentially impact certain other kinases implicated in certain neurodegenerative diseases. Id., Hanger, et al., Trends in Mol. Med., 15(3), 112-119 (2009). These certain other kinases potentially implicated in amyloid-β production include ARG, PDGFR, Src, and c-kit. Netzer, et al., PNAS, 100(21): 12444-12449 (2003). Despite the apparent promise of STI-571 in in vitro and certain in vivo models, its clinical promise for humans is stymied by its inability to cross the blood-brain barrier, which is viewed as being necessary to improve the likelihood of therapeutic benefit despite certain contradictory views. Sutcliffe, et al., J. of Neuro. Res., 89:808-814 (2011).

In the context of targeted therapies, it has been found that the microtubule-associated protein tau is integral to the pathogenesis of AD and related disorders termed tauopathies. Strategies for targeting tau in neurodegenerative disease include (i) reducing tau phosphorylation through inhibition of specific protein kinases; (ii) disaggregating tau inclusions; and (iii) tau immunotherapy with (i) being the preferred approach. Hanger, et al., Trends in Mol. Med., 15(3), 112-119 (2009). Specific protein kinases implicated in the reduction of tau phosphorylation include glycogen synthase kinase-3 (GSK-3), cyclin-dependent kinase-5 (cdk5), extracellular signal-regulated kinase-2 (ERK2), cyclic AMP-dependent protein kinase (PKA), casein kinase 1 (CK1), MAPK and JNK. These kinases and the kinases implicated with AD such as Fyn, Syk and c-Abl are also of interest in ability to phosphorylate tau at Y18, Y197 and Y394, respectively. Id.

While the etiology of these neurodegenerative diseases is not fully understood, it is believed that there is a need for inhibitors of Abl that also exhibit activity against other kinases such as src, PDGFR and/or c-kit. Such inhibitors would be attractive for development. Applicant's own WO 2007/075869, which is hereby incorporated herein by reference for all purposes, discloses certain compounds that are targeted tyrosine kinase inhibitors. One notable targeted TKI is ponatinib, which is currently the subject of a clinical trial to determine the efficacy of ponatinib in patients with chronic myeloid leukemia (CML) in chronic phase (CP), accelerated phase (AP) or blast phase (BP) or with Ph positive (Ph+) acute lymphoblastic leukemia (ALL) who either are resistant or intolerant to either dasatinib or nilotinib, or have the T315I mutation of Bcr-Abl (clinical trials.gov identifier NCT01207440). WO 2007/075869 does not explicitly mention using such targeted TKI's for the treatment of AD or other neurodegenerative diseases.

Applicant's own WO 2011/053938, which is hereby incorporated herein by reference for all purposes, discloses in the context of methods for treating cancer that these compounds have a wide range of kinase activity beyond the initial focus on Abl inhibition. For instance, these compounds demonstrate potency against PDGFR, c-SRC, and certain other kinases shown at Table 8.

### SUMMARY

It has been unexpectedly discovered that certain targeted TKI's cross the blood brain barrier and are useful in the inhibition of β-amyloid production and accordingly for the treatment of AD. In addition, such targeted TKI's are useful for targeting tau and accordingly for the treatment of neurodegenerative diseases, including Parkinson's disease, Alzheimer's disease and other neurodegenerative diseases as disclosed herein.

In one aspect, this disclosure provides methods for treating or preventing a neurodegenerative condition in a subject in need thereof by administering to the subject an effective amount of a targeted TKI of Formula I:

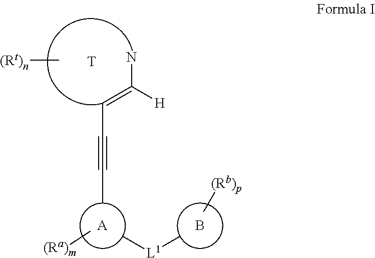

or a tautomer, or an individual isomer or a mixture of isomers thereof wherein:

Ring T is a 5-membered heteroaryl ring containing 1 or 2 nitrogens with the remaining ring atoms being carbon, substituted on at least two ring atoms with R^{t} groups, at least two of which being located on adjacent ring atoms, and, together with the atoms to which they are attached, forming a saturated, partially saturated or unsaturated 5- or 6-membered ring (Ring E), containing 0-3 heteroatoms selected from O, N, and S and being optionally substituted with 1-4 R^{e} groups;

Ring A is a 5- or 6-membered aryl or heteroaryl ring and is optionally substituted with 1-4 R^{a} groups;
Ring B is a 5- or 6-membered aryl or heteroaryl ring;
L¹ is selected from NR¹C(O), C(O)NR¹, NR¹C(O)O, NR¹C(O)NR¹, and OC(O)NR¹;
each occurrence of R^{a}, R^{b} and R^{t} is independently selected from the group consisting of halo, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;
R^{e}, at each occurrence, is independently selected from the group consisting of halo, ═O, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;
R¹, R² and R³ are independently selected from H, alkyl, alkenyl, alkynyl, cycloalkyl, cycloalkenyl, cycloalkynyl, aryl, heterocyclic and heteroaryl;
alternatively, R² and R³, taken together with the atom to which they are attached, form a 5- or 6-membered saturated, partially saturated or unsaturated ring, which can be optionally substituted and which contains 0-2 heteroatoms selected from N, O and S(O)ᵣ;
each occurrence of R⁴ is independently selected from alkyl, alkenyl, alkynyl, cycloalkyl, cycloalkenyl, cycloalkynyl, aryl, heterocyclic and heteroaryl;
each of the alkyl, alkenyl, alkynyl, cycloalkyl, cycloalkenyl, cycloalkynyl, aryl, heterocyclic and heteroaryl moieties is optionally substituted;
m is 0, 1, 2, 3 or 4;
n is 2 or 3;
p is 0, 1, 2, 3, 4 or 5; and,
r is 0, 1 or 2;

or a pharmaceutically acceptable salt, solvate or hydrate thereof.

In another aspect, this disclosure provides methods for treating or preventing Alzheimer's disease in a subject in need thereof by administering to the subject a targeted tyrosine kinase inhibitor in an amount sufficient to reduce the activity of γ-secretase in the brain of the subject, wherein the targeted tyrosine kinase inhibitor is a compound of Formula I:

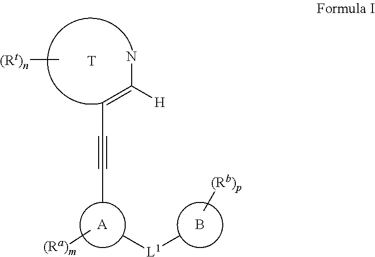

or a tautomer, or an individual isomer or a mixture of isomers thereof wherein:
Ring T is a 5-membered heteroaryl ring containing 1 or 2 nitrogens with the remaining ring atoms being carbon, substituted on at least two ring atoms with R^{t} groups, at least two of which being located on adjacent ring atoms, and, together with the atoms to which they are attached, forming a saturated, partially saturated or unsaturated 5- or 6-membered ring (Ring E), containing 0-3 heteroatoms selected from O, N, and S and being optionally substituted with 1-4 R^{e} groups;
Ring A is a 5- or 6-membered aryl or heteroaryl ring and is optionally substituted with 1-4 R^{a} groups;
Ring B is a 5- or 6-membered aryl or heteroaryl ring;
L¹ is selected from NR¹C(O), C(O)NR¹, NR¹C(O)O, NR¹C(O)NR¹, and OC(O)NR¹;
each occurrence of R^{a}, R^{b} and R^{t} is independently selected from the group consisting of halo, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;
R^{e}, at each occurrence, is independently selected from the group consisting of halo, ═O, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;
R¹, R² and R³ are independently selected from H, alkyl, alkenyl, alkynyl, cycloalkyl, cycloalkenyl, cycloalkynyl, aryl, heterocyclic and heteroaryl;
alternatively, R² and R³, taken together with the atom to which they are attached, form a 5- or 6-membered saturated, partially saturated or unsaturated ring, which can be optionally substituted and which contains 0-2 heteroatoms selected from N, O and S(O)ᵣ;
each occurrence of R⁴ is independently selected from alkyl, alkenyl, alkynyl, cycloalkyl, cycloalkenyl, cycloalkynyl, aryl, heterocyclic and heteroaryl;
each of the alkyl, alkenyl, alkynyl, cycloalkyl, cycloalkenyl, cycloalkynyl, aryl, heterocyclic and heteroaryl moieties is optionally substituted;
m is 0, 1, 2, 3 or 4;
n is 2 or 3;
p is 0, 1, 2, 3, 4 or 5; and,
r is 0, 1 or 2;

or a pharmaceutically acceptable salt, solvate or hydrate thereof.

In another aspect, this disclosure provides pharmaceutical compositions for treating or preventing a neurodegenerative condition in a subject in need thereof comprising an effective amount of a targeted tyrosine kinase inhibitor, wherein the targeted TKI is a compound of Formula I:

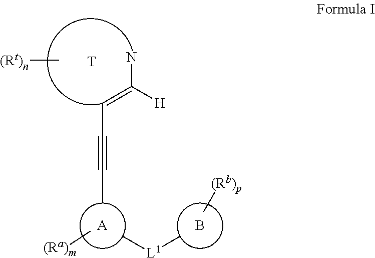

or a tautomer, or an individual isomer or a mixture of isomers thereof wherein:
Ring T is a 5-membered heteroaryl ring containing 1 or 2 nitrogens with the remaining ring atoms being carbon, substituted on at least two ring atoms with R^{t} groups, at least two of which being located on adjacent ring atoms, and, together with the atoms to which they are attached, forming a saturated, partially saturated or unsaturated 5- or 6-membered ring (Ring E), containing 0-3 heteroatoms selected from O, N, and S and being optionally substituted with 1-4 R^{e} groups;
Ring A is a 5- or 6-membered aryl or heteroaryl ring and is optionally substituted with 1-4 R^{a} groups;
Ring B is a 5- or 6-membered aryl or heteroaryl ring;
L¹ is selected from NR¹C(O), C(O)NR¹, NR¹C(O)O, NR¹C(O)NR¹, and OC(O)NR¹;
each occurrence of R^{a}, R^{b} and R^{t} is independently selected from the group consisting of halo, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ₙR², —SO₂NR²R³ and —NR²SO₂NR²R³, wherein each Y is independently a bond, —O—, —S— or —NR³—;
R^{e}, at each occurrence, is independently selected from the group consisting of halo, ═O, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;
R¹, R² and R³ are independently selected from H, alkyl, alkenyl, alkynyl, cycloalkyl, cycloalkenyl, cycloalkynyl, aryl, heterocyclic and heteroaryl;
alternatively, R² and R³, taken together with the atom to which they are attached, form a 5- or 6-membered saturated, partially saturated or unsaturated ring, which can be optionally substituted and which contains 0-2 heteroatoms selected from N, O and S(O)ᵣ;
each occurrence of R⁴ is independently selected from alkyl, alkenyl, alkynyl, cycloalkyl, cycloalkenyl, cycloalkynyl, aryl, heterocyclic and heteroaryl;
each of the alkyl, alkenyl, alkynyl, cycloalkyl, cycloalkenyl, cycloalkynyl, aryl, heterocyclic and heteroaryl moieties is optionally substituted;
m is 0, 1, 2, 3 or 4;
n is 2 or 3;
p is 0, 1, 2, 3, 4 or 5; and,
r is 0, 1 or 2;

or a pharmaceutically acceptable salt, solvate or hydrate thereof; and

a pharmaceutically acceptable carrier.

In another aspect, this disclosure provides kits including: (a) a presently disclosed targeted tyrosine kinase inhibitor, and (b) instructions for administering the targeted TKI to a subject diagnosed with or at risk of developing a neurodegenerative disease. The targeted tyrosine kinase inhibitor can be formulated for administration according to any of the dosing regimens described herein. As noted at the outset, the targeted tyrosine kinase inhibitor used in the various embodiments of the invention may be in the form of its free base or a pharmaceutically acceptable salt thereof.

In certain embodiments of any of the foregoing methods or pharmaceutical compositions in the compound of Formula I, the targeted tyrosine kinase inhibitor is a compound selected from the group consisting of:
N-(3-(1H-imidazol-1-yl)-5-(trifluoromethyl)phenyl)-3-(imidazo[1,2-a]pyrazin-3-ylethynyl)-4-methylbenzamide;
3-(Imidazo[1,2-a]pyrazin-3-ylethynyl)-4-methyl-N-(4-((4-methylpiperazin-1-yl)methyl)-3-(trifluoromethyl)phenyl)benzamide;
N-(3-(2-((dimethylamino)methyl)-1H-imidazol-1-yl)-5-(trifluoromethyl)phenyl)-3-(imidazo[1,2-a]pyrazin-3-ylethynyl)-4-methylbenzamide;
3-(Imidazo[1,2-a]pyridin-3-ylethynyl)-4-methyl-N-(3-(4-methyl-1H-imidazol-1-yl)-5-(trifluoromethyl)phenyl)benzamide;
N-(3-(1H-imidazol-1-yl)-5-(trifluoromethyl)phenyl)-3-(imidazo[1,2-a]pyridin-3-ylethynyl)-4-methylbenzamide;
3-(Imidazo[1,2-a]pyridin-3-ylethynyl)-4-methyl-N-(4-(trifluoromethyl)pyridin-2-yl)benzamide;
N-(5-tert-butylisoxazol-3-yl)-3-(imidazo[1,2-a]pyridin-3-ylethynyl)-4-methylbenzamide;
3-(Imidazo[1,2-a]pyridin-3-ylethynyl)-4-methyl-N-(4-((4-methylpiperazin-1-yl)methyl)-3-(trifluoromethyl)phenyl)benzamide;
N-(3-(2-((dimethylamino)methyl)-1H-imidazol-1-yl)-5-(trifluoromethyl)phenyl)-3-(imidazo[1,2-a]pyridin-3-ylethynyl)-4-methylbenzamide;
3-((8-Acetamidoimidazo[1,2-a]pyridin-3-yl)ethynyl)-4-methyl-N-(4-(trifluoromethyl)pyridin-2-yl)benzamide;
N-(3-(1H-imidazol-1-yl)-5-(trifluoromethyl)phenyl)-3-((8-acetamidoimidazo[1,2-a]pyridin-3-yl)ethynyl)-4-methylbenzamide;
4-Methyl-3-((8-(4-(methylsulfonyl)phenylamino)imidazo[1,2-a]pyridin-3-yl)ethynyl)-N-(4-(trifluoromethyl)pyridin-2-yl)benzamide;
4-methyl-3-((8-(4-sulfamoylphenylamino)imidazo[1,2-a]pyridin-3-yl)ethynyl)-N-(4-(trifluoromethyl)pyridin-2-yl)benzamide;
(R)—N-(4-((3-(Dimethylamino)pyrrolidin-1-yl)methyl)-3-(trifluoromethyl)phenyl)-3-(imidazo[1,2-b]pyridazin-3-ylethynyl)-4-methylbenzamide;
N-(3-(Imidazo[1,2-b]pyridazin-3-ylethynyl)-4-methylphenyl)-4-((4-methylpiperazin-1-yl)methyl)-3-(trifluoromethyl)benzamide;
3-(Imidazo[1,2-b]pyridazin-3-ylethynyl)-4-methyl-N-(4-((4-methylpiperazin-1-yl)methyl)-3-(trifluoromethyl)phenyl)benzamide;
N-(3-Chloro-4-((4-methylpiperazin-1-yl)methyl)phenyl)-3-(imidazo[1,2-b]pyridazin-3-yl ethynyl)-4-methylbenzamide;
N-(3-Cyclopropyl-4-((4-methylpiperazin-1-yl)methyl)phenyl)-3-(imidazo[1,2-b]pyridazin-3-ylethynyl)-4-methylbenzamide;
3-(Imidazo[1,2-b]pyridazin-3-ylethynyl)-N-(4-((4-methylpiperazin-1-yl)methyl)-3-(trifluoromethyl)phenyl)benzamide;
N-(4-((4-(2-Hydroxyethyl)piperazin-1-yl)methyl)-3-(trifluoromethyl)phenyl)-3-(imidazo[1,2-b]pyridazin-3-ylethynyl)-4-methylbenzamide; and
3-(Imidazo[1,2-b]pyridazin-3-ylethynyl)-4-methyl-N-(4-(piperazin-1-ylmethyl)-3-(trifluoromethyl)phenyl)benzamide,
   or a pharmaceutically acceptable salt thereof.

Additional features and advantages of the methods and pharmaceutical compositions disclosed herein will be apparent from the following detailed description.

### DETAILED DESCRIPTION

### Definitions

In reading this document, the following information and definitions apply unless otherwise indicated.

As used herein, a “targeted tyrosine kinase inhibitor” or “targeted TKI” means a compound of Formula I as disclosed herein that is active against at least one kinase selected from the group consisting of Abl, PDGFR, ARG, fyn, syk, c-kit and src, as determined by an appropriate in vitro kinase assay. In general, a targeted TKI is said to be active against a relevant kinase if it has an IC₅₀ less than 1 μM in such in vitro kinase assay. A typical in vitro kinase assay used to determine the activity for Abl, PDGFR, ARG, c-kit and src is described in O'Hare et al, Cancer Cell 16:401-412 (2009) and Supplemental Data.

As used herein, the term “ponatinib” means 3-(imidazo[1,2-b]pyridazin-3-ylethynyl)-4-methyl-N-(4-((4-methylpiperazin-1-yl)-methyl-3-(trifluoromethyl)phenyl)benzamide (as shown in Example 16 herein) and having the chemical structure depicted below:

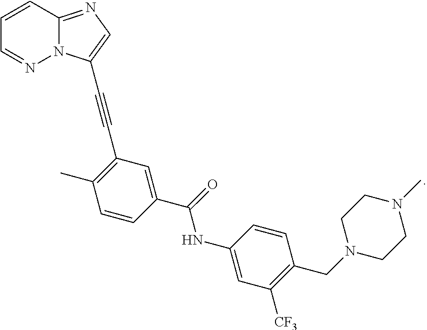 [SMILES: Cc1ccc(C(=O)Nc2ccc(CN3CCN(C)CC3)c(C(F)(F)F)c2)cc1C#Cc1cnc2cccnn12]

The term ponatinib refers only to its free base unless a pharmaceutically acceptable salt (such as ponatinib HCl) is explicitly mentioned.

As used herein, the term “mean steady state trough concentration” means the average plasma concentration of a compound disclosed herein observed for a group of subjects as part of a dosing regimen for a therapy of the invention administered over a period of time sufficient to produce steady state pharmacokinetics (i.e., a period of 23 days of daily dosing), wherein the mean trough concentration is the average circulating concentration over all of the subjects at a time just prior to (i.e., within 1 hour of) the next scheduled administration in the regimen (e.g., for a daily regimen the trough concentration is measured about 24 hours after an administration of a compound disclosed herein and just prior to the subsequent daily administration).

As used herein, the terms “administration” or “administering” mean a route of administration for a compound disclosed herein. Exemplary routes of administration include, but are not limited to, oral, intravenous, intraperitoneal, intraarterial, and intramuscular. The preferred route of administration can vary depending on various factors, e.g., the components of the pharmaceutical composition comprising a compound disclosed herein, site of the potential or actual disease and severity of disease. While ponatinib will generally be administered per orally, other routes of administration can be useful in carrying out the methods of the invention.

As used herein, the term “unit dosage form” means a physically discrete unit containing a predetermined quantity of a compound disclosed herein that is suitable for administration. Exemplary unit dosage forms include, but are not limited to, a pill, tablet, caplet, hard capsule or soft capsule.

As used herein, the term “pharmaceutically acceptable salt” means salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, phosphonates and other types of compounds, are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the isolation and purification of the compounds of the invention, or separately by reacting the free base or free acid of a compound of the invention with a suitable base or acid, respectively. Examples of pharmaceutically acceptable, nontoxic acid addition salts of a compound disclosed herein are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methane-sulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As used herein, the term “pharmaceutically acceptable carrier” or “pharmaceutically acceptable adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound. Pharmaceutically acceptable carriers, adjuvants and vehicles that can be used in the pharmaceutical compositions of this invention include, but are not limited to, ion exchangers, alumina, aluminum stearate, lecithin, self emulsifying drug delivery systems (SEDDS) such as d-atocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty, acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as u-, P-, and y-cyclodextrin, or chemically modified derivatives such as hydroxyalkylcyclodextrins, including 2 and 3-hydroxypropyl-cyclodextrins, or other solubilized derivatives may also be advantageously used to enhance delivery of compounds of the formulae described herein.

As used herein, the terms “treatment” or “treating” mean: (1) improving or stabilizing the subject's condition or disease or (2) preventing or relieving the development or worsening of symptoms associated with the subject's condition or disease.

As used herein, the terms “amount effective” or “effective amount” mean the amount of a compound disclosed herein that when administered to a subject for treating a disease, is sufficient to effect such treatment of the disease. Any improvement in the patient is considered sufficient to achieve treatment. An effective amount of a compound disclosed herein, used for the treatment of a neurodegenerative disease can vary depending upon the manner of administration, the age, body weight, and general health of the patient. Ultimately, the prescribers or researchers will decide the appropriate amount and dosage regimen.

As used herein, the term “tau pathology” means a neurodegenerative condition characterized by intracellular inclusions, such as flame-shaped or globular neurofibrillary tangles and/or neuropil threads (fine filamentous structures found primarily in dendrites), which include insoluble phosphorylated forms of tau. Tau pathologies include Alzheimer's disease, progressive supranuclear palsy, Pick's disease, corticobasal degeneration and fronto-temporal dementia linked to chromosome 17 with parkinsonism (FTDP-17T).

As used herein, the terms “neurodegenerative condition” and “neurodegenerative disease” are used interchangeably in this document and mean diseases of the nervous system (e.g., the central nervous system or peripheral nervous system) characterized by abnormal cell death. Examples of neurodegenerative conditions include Alzheimer disease, Down's syndrome, frontotemporal dementia, progressive supranuclear palsy, Pick's disease, Niemann-Pick disease, Parkinson's disease, Huntington's disease (HD), dentatorubropallidoluysian atrophy, Kennedy's disease (also referred to as spinobulbar muscular atrophy), and spinocerebellar ataxia (e.g., type 1, type 2, type 3 (also referred to as Machado-Joseph disease), type 6, type 7, and type 17)), fragile X (Rett's) syndrome, fragile XE mental retardation, Friedreich's ataxia, myotonic dystrophy, spinocerebellar ataxia type 8, and spinocerebellar ataxia type 12, Alexander disease, Alper's disease, amyotrophic lateral sclerosis, ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, ischemia stroke, Krabbe disease, Lewy body dementia, multiple sclerosis, multiple system atrophy, Pelizaeus-Merzbacher disease, Pick's disease, primary lateral sclerosis, Refsum's disease, Sandhoff disease, Schilder's disease, spinal cord injury, spinal muscular atrophy, Steele-Richardson-Olszewski disease, and Tabes dorsalis.

As used herein, the term “neurodegenerative conditions associated with mitochondrial dysfunction” means a neurodegenerative condition that is characterized by or implicated by mitochondrial dysfunction. Exemplary neurodegenerative conditions associated with mitochondrial dysfunction include, without limitation, Friedrich's ataxia, amyotrophic lateral sclerosis, mitochondrial myopathy, encephalopathy, lactacidosis, stroke (MELAS), myoclonic epilepsy with ragged red fibers (MERFF), epilepsy, Parkinson's disease, Alzheimer's disease, and Huntington's Disease.

The terms “subject” and “patient” are used herein interchangeably. They refer to a human or another mammal (e.g., mouse, rat, rabbit, dog, cat, cattle, swine, sheep, horse or primate) that can be afflicted with or is susceptible to a disease or disorder (e.g., AD) but may or may not have the disease or disorder. In certain embodiments, the subject is a human being.

As used herein, the term “alkyl” is intended to include linear (i.e., unbranched or acyclic), branched, cyclic, or polycyclic non aromatic hydrocarbon groups, which are optionally substituted with one or more functional groups. Unless otherwise specified, “alkyl” groups contain one to eight, and preferably one to six carbon atoms. C₁₋₆ alkyl, is intended to include C₁, C₂, C₃, C₄, C₅, and C₆ alkyl groups. Lower alkyl refers to alkyl groups containing 1 to 6 carbon atoms. Examples of Alkyl include, but are not limited to, methyl, ethyl, n-propyl, isopropyl, cyclopropyl, butyl, isobutyl, sec-butyl, tert-butyl, cyclobutyl, pentyl, isopentyl tert-pentyl, cyclopentyl, hexyl, isohexyl, cyclohexyl, etc. Alkyl may be substituted or unsubstituted. Illustrative substituted alkyl groups include, but are not limited to, fluoromethyl, difluoromethyl, trifluoromethyl, 2-fluoroethyl, 3-fluoropropyl, hydroxymethyl, 2-hydroxyethyl, 3-hydroxypropyl, benzyl, substituted benzyl, phenethyl, substituted phenethyl, etc.

As used herein, the term “Alkoxy” means a subset of alkyl in which an alkyl group as defined above with the indicated number of carbons attached through an oxygen bridge. For example, “alkoxy” refers to groups —O-alkyl, wherein the alkyl group contains 1 to 8 carbons atoms of a linear, branched, cyclic configuration. Examples of “alkoxy” include, but are not limited to, methoxy, ethoxy, n-propoxy, i-propoxy, t-butoxy, n-butoxy, s-pentoxy and the like.

As used herein, the term “Haloalkyl” is intended to include both branched and linear chain saturated hydrocarbon having one or more carbon substituted with a Halogen. Examples of haloalkyl, include, but are not limited to, trifluoromethyl, trichloromethyl, pentafluoroethyl and the like.

As used herein, the term “alkenyl” is intended to include hydrocarbon chains of linear, branched, or cyclic configuration having one or more unsaturated Carbon-carbon bonds that may occur in any stable point along the chain or cycle. Unless otherwise specified, “alkenyl” refers to groups usually having two to eight, often two to six carbon atoms. For example, “alkenyl” may refer to prop-2-enyl, but-2-enyl, but-3-enyl, 2-methylprop-2-enyl, hex-2-enyl, hex-5-enyl, 2,3-dimethylbut-2-enyl, and the like. Furthermore, alkenyl groups may be substituted or unsubstituted.

As used herein, the term “alkynyl” is intended to include hydrocarbon chains of either linear or branched configuration, having one or more carbon-carbon triple bond that may occur in any stable point along the chain. Unless otherwise specified, “alkynyl” groups refer refers to groups having two to eight, preferably two to six carbons. Examples of “alkynyl” include, but are not limited to prop-2-ynyl, but-2-ynyl, but-3-ynyl, pent-2-ynyl, 3-methylpent-4-ynyl, hex-2-ynyl, hex-5-ynyl, etc. Furthermore, alkynyl groups may be substituted or unsubstituted.

As used herein, the term “Cycloalkyl” is a subset of alkyl and includes any stable cyclic or polycyclic hydrocarbon groups of from 3 to 13 carbon atoms, any of which is saturated. Examples of such cycloalkyl include, but are not limited to cyclopropyl, norbornyl, [2.2.2]bicyclooctane, [4.4.0]bicyclodecane, and the like, which, as in the case of other alkyl moieties, may optionally be substituted. The term “cycloalkyl” may be used interchangeably with the term “carbocycle”.

As used herein, the term “Cycloalkenyl” is a subset of alkenyl and includes any stable cyclic or polycyclic hydrocarbon groups of from 3 to 13 carbon atoms, preferably from 5 to 8 carbon atoms, which contains one or more unsaturated carbon-carbon double bonds that may occur in any point along the cycle. Examples of such cycloalkenyl include, but are not limited to cyclopentenyl, cyclohexenyl and the like.

As used herein, the term “Cycloalkynyl” is a subset of alkynyl and includes any stable cyclic or polycyclic hydrocarbon groups of from 5 to 13 carbon atoms, which contains one or more unsaturated carbon-carbon triple bonds that may occur in any point along the cycle. As in the case of other alkenyl and alkynyl moieties, cycloalkenyl and cycloalkynyl may optionally be substituted.

As used herein, the terms “Heterocycle”, “heterocyclyl”, or “heterocyclic” as used herein refers to non-aromatic ring systems having five to fourteen ring atoms, preferably five to ten, in which one or more ring carbons, preferably one to four, are each replaced by a heteroatom such as N, O, or S. Non-limiting examples of heterocyclic rings include 3-1H-benzimidazol-2-one, (1-substituted)-2-oxo-benzimidazol-3-yl, 2-tetrahydrofuranyl, 3-tetrahydrofuranyl, 2-tetrahydrothiophenyl, 3-tetrahydrothiophenyl, 2-morpholinyl, 3-morpholinyl, 4-morpholinyl, 2-thiomorpholinyl, 3-thiomorpholinyl, 4-thiomorpholinyl, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl, 4-thiazolidinyl, diazolonyl, N-substituted diazolonyl, 1-phthalimidinyl, benzoxanyl, benzopyrrolidinyl, benzopiperidinyl, benzoxolanyl, benzothiolanyl, and benzothianyl. Also included within the scope of the term “heterocyclyl” or “heterocyclic”, as it is used herein, is a group in which a non-aromatic heteroatom-containing ring is fused to one or more aromatic or non-aromatic rings, such as in an indolinyl, chromanyl, phenanthridinyl, or tetrahydroquinolinyl, where the radical or point of attachment is on the non-aromatic heteroatom-containing ring. The term “heterocycle”, “heterocyclyl”, or “heterocyclic” whether saturated or partially unsaturated, also refers to rings that are optionally substituted.

As used herein, the term “aryl” used alone or as part of a larger moiety as in “aralkyl”, “aralkoxy”, or “aryloxy-alkyl”, refers to aromatic ring groups having six to fourteen ring atoms, such as phenyl, 1-naphthyl, 2-naphthyl, 1-anthracyl and 2-anthracyl. An “aryl” ring may contain one or more substituents. The term “aryl” may be used interchangeably with the term “aryl ring”. “Aryl” also includes fused polycyclic aromatic ring systems in which an aromatic ring is fused to one or more rings. Non-limiting examples of useful aryl ring groups include phenyl, hydroxyphenyl, halophenyl, alkoxyphenyl, dialkoxyphenyl, trialkoxyphenyl, alkylenedioxyphenyl, naphthyl, phenanthryl, anthryl, phenanthro and the like, as well as 1-naphthyl, 2-naphthyl, 1-anthracyl and 2-anthracyl. Also included within the scope of the term “aryl”, as it is used herein, is a group in which an aromatic ring is fused to one or more non-aromatic rings, such as in a indanyl, phenanthridinyl, or tetrahydronaphthyl, where the radical or point of attachment is on the aromatic ring.

As used herein, the term “heteroaryl” as used herein refers to stable heterocyclic, and polyheterocyclic aromatic moieties having 5-14 ring atoms. Heteroaryl groups may be substituted or unsubstituted and may comprise one or more rings. Examples of typical heteroaryl rings include 5-membered monocyclic ring groups such as thienyl, pyrrolyl, imidazolyl, pyrazolyl, furyl, isothiazolyl, furazanyl, isoxazolyl, thiazolyl and the like; 6-membered monocyclic groups such as pyridyl, pyrazinyl, pyrimidinyl, pyridazinyl, triazinyl and the like; and polycyclic heterocyclic ring groups such as benzo[b]thienyl, naphtho[2,3-b]thienyl, thianthrenyl, isobenzofuranyl, chromenyl, xanthenyl, phenoxathienyl, indolizinyl, isoindolyl, indolyl, indazolyl, purinyl, isoquinolyl, quinolyl, phthalazinyl, naphthyridinyl, quinoxalinyl, quinazolinyl, benzothiazole, benzimidazole, tetrahydroquinoline cinnolinyl, pteridinyl, carbazolyl, beta-carbolinyl, phenanthridinyl, acridinyl, perimidinyl, phenanthrolinyl, phenazinyl, isothiazolyl, phenothiazinyl, phenoxazinyl, and the like (see e.g. Katritzky, Handbook of Heterocyclic Chemistry). Further specific examples of heteroaryl rings include 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 1-pyrrolyl, 2-pyrrolyl, 3-pyrrolyl, 2-pyridyl, 3-pyridyl, 4-pyridyl, 2-pyrimidyl, 4-pyrimidyl, 5-pyrimidyl, 3-pyridazinyl, 2-thiazolyl, 4-thiazolyl, 5-thiazolyl, 5-tetrazolyl, 2-triazolyl, 5-triazolyl, 2-thienyl, 3-thienyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, indolyl, isoindolyl, acridinyl, or benzoisoxazolyl. Heteroaryl groups further include a group in which a heteroaromatic ring is fused to one or more aromatic or nonaromatic rings where the radical or point of attachment is on the heteroaromatic ring. Examples include tetrahydroquinoline, tetrahydroisoquinoline, and pyrido[3,4-d]pyrimidinyl, imidazo[1,2-a]pyrimidyl, imidazo[1,2-a]pyrazinyl, imidazo[1,2-a]pyiridinyl, imidazo[1,2-a]pyrimidyl, pyrazolo[1,5-a][1,3,5]triazinyl, pyrazolo[1,5-c]pyrimidyl, imidazo[1,2-b]pyridazinyl, imidazo[1,5-a]pyrimidyl, pyrazolo[1,5-b][1,2,4]triazine, quinolyl, isoquinolyl, quinoxalyl, imidazotriazinyl, pyrrolo[2,3-d]pyrimidyl, triazolopyrimidyl, pyridopyrazinyl. The term “heteroaryl” also refers to rings that are optionally substituted. The term “heteroaryl” may be used interchangeably with the term “heteroaryl ring” or the term “heteroaromatic”.

### Methods

The ability of a compound to accumulate to pharmacologically relevant levels in the brain is a function of a series of factors. A partial list of such factors includes the ability of the compound to diffuse away from any protein binding in the blood, cross the blood brain barrier to enter the brain, avoid active removal by the p-Glycoprotein efflux pump and survive metabolic or other clearance mechanisms in the brain. Those individual characteristics, let alone their net cumulative net effect, cannot yet be predicted with useful precision and confidence based on the chemical structure of a given compound and therefore depend on empirical determination. Unfavorable behavior in any one of those characteristics can rule out effective accumulation in brain.

In pharmacokinetic experiments in rodents, we have found that the potent targeted tyrosine kinase inhibitor, ponatinib, not only reaches the brain and accumulates, but actually accumulates in the brain to levels between two- and three-fold higher than in blood. This was an unexpected fortuitous finding. The very favorable accumulation of ponatinib in brain combined with its significant inhibitory potency against kinases such as Abl, PDGFR, c-kit and src permits delivery of pharmacologically relevant concentrations of drug to the brain, e.g., at levels effective to inhibit β-amyloid production or regulate tau phosphorylation in the brain which has been associated with the development of neurodegenerative disorders including AD. For a kinase inhibition profile of ponatinib, including a partial list of kinases inhibited with inter alia an IC₅₀ below 50 nM and of kinases inhibited with an 1050 below 10 nM, see O'Hare et al, Cancer Cell 16:401-412 (2009)(Supplemental Data) and the examples disclosed herein. While not limiting this invention to any one mechanism of action, the ability of this potent agent to accumulate as it does in brain makes ponatinib a very attractive agent for treating neurodegenerative conditions including AD.

As discussed herein, this disclosure provides a method for treating neurodegenerative disorders by administering to a patient in need thereof an effective amount of a compound of Formula I such as ponatinib or a pharmaceutically acceptable salt thereof.

In certain embodiments, this disclosure provides a method for treating or inhibiting the development of neurodegenerative disorders including Alzheimer's disease including the steps of: (a) providing a subject having, or at risk of, neurodegenerative disorders including Alzheimer's disease; and (b) administering to the subject a compound of Formula I in an amount effective to treat, or inhibit the development of, neurodegenerative disorders including Alzheimer's disease.

In certain embodiments, this disclosure provides a method for treating or inhibiting the development of Alzheimer's disease including the steps of: (a) providing a subject having or at risk of developing Alzheimer's disease; and (b) administering to the subject a compound of Formula I, or a pharmaceutically acceptable salt thereof, in an amount sufficient to reduce the activity of γ-secretase in the brain of the subject.

In any of the above methods, the neurodegenerative condition can be Parkinson's disease, Alzheimer's disease, multiple sclerosis, or any other neurodegenerative disease described herein. In particular embodiments, the neurodegenerative condition is associated with mitochondrial dysfunction (e.g., Friedrich's ataxia, amyotrophic lateral sclerosis, mitochondrial myopathy, encephalopathy, lactacidosis, stroke (MELAS), myoclonic epilepsy with ragged red fibers (MERFF), epilepsy, or Huntington's Disease). In some embodiments, the neurodegenerative condition is a tau pathology (e.g., progressive supranuclear palsy, Pick's disease, corticobasal degeneration, or fronto-temporal dementia linked to chromosome 17 with parkinsonism).

### Therapy

The method of this invention may be carried out at the patient's residence, the doctor's office, a clinic, a hospital's outpatient department, or elsewhere. Treatment may be initiated at a hospital so that the doctor can observe the therapy's effects directly and make any adjustments that may be needed. The duration of the therapy depends on the age and condition of the patient, the stage of the patient's a neurodegenerative condition, and how the patient responds to the treatment. Additionally, a person at greater risk of developing a neurodegenerative condition (e.g., a person who is genetically predisposed) may receive ponatinib therapy to inhibit or delay onset, progression or symptoms of the disease.

The method of this invention may be used to treat neurodegenerative conditions that have been linked to mitochondrial dysfunction. Many progressive neurological diseases have been linked to destruction of neurons by mitochondrial apoptosis. Friedrich's ataxia results from a genetic defect in the frataxin gene, which is involved in mitochondrial iron transport (Babcock et al., Science 276:1709 (1997)); human deafness dystonia results from a defect in a small component of the mitochondrial protein import machinery (Koehler et al., Proc. Natl. Acad. Sci. USA 96:2141 (1999)); one well-characterized cause of amyotrophic lateral sclerosis is deficiency in Cu—Zn superoxide dismutase, which is located in the mitochondrial intermembrane space as well as the cytoplasm (Deng et al., Science 261:1047 (1993)). The discovery that several environmental toxins cause Parkinsonism by inhibiting respiratory complex I and promoting the generation of reactive oxygen species has made this complex a focus for research on the basis of Parkinson's disease (Dawson et al., Science 302:819 (2003)). More recently, the mitochondrial protein encoded by PINK 1 has provided a direct link between mitochondria and Parkinson's disease (Valente et al., Science 304:1158 (2004)). Alzheimer's disease is also linked to mitochondrial toxicity through the mitochondrial protein ABAD, a target of amyloid (Lustbader et al., Science 304:448 (2004)). Huntington's Disease has been associated with defects in energy metabolism that appear to be widespread, affecting both the brain and peripheral tissues, and arising from mitochondrial dysfunction (Leegwater-Kim et al., NeuroRx 1:128 (2004)).

The method of this invention can be used to treat neurodegenerative conditions characterized by the accumulation of misfolded proteins including, without limitation, Parkinson's disease, Alzheimer's disease and tau pathologies.

Alzheimer's Disease

AD is characterized by a progressive decline in cognitive functions. Neuropathologies of the disease include the accumulation of tangles, β-amyloid containing plaques, dystrophic neurites, and loss of synapses and neurons (Selkoe, D. et al., Alzheimer's Disease, Ed2. Terry R. et al., eds. pg. 293-310, 1999. Philadelphia: Lippincott, Williams and Wilkins), but these pathologies are preceded by deficits in spatial and long-term memory generation (Vitolo et al., Proc Natl Acad Sci USA. 99:13217 (2002)). AD exists as sporadic as well as heritable familial forms. While the sporadic version is more prevalent, study of familial AD may provide insight into sporadic AD since pathologies of both are similar. Familial AD results from mutations in the presenilin genes, an essential component of the γ-secretase enzyme complex; or amyloid precursor proteins, a substrate of γ-secretase and the precursor of β-amyloid. These mutations result in the accumulation of β-amyloid protein plaques in the brains of affected individuals.

One set of criteria for the diagnosis of AD includes: (i) dementia established by examination and objective testing; (ii) deficits in two or more cognitive areas; (iii) progressive worsening of memory and other cognitive functions; (iv) no disturbance in consciousness; and (v) Onset between ages 40 and 90.

Parkinson's Disease

The presence of one or more of the following symptoms may be used as part of a Parkinson's Disease (PD) diagnosis: trembling, e.g., an involuntary, rhythmic tremor of one arm or one leg; muscular rigidity, stiffness, or discomfort; general slowness in any of the activities of daily living, e.g., akinesia or bradykinesia; difficulty with walking, balance, or posture; alteration in handwriting; emotional changes; memory loss; speech problems; and difficulty sleeping. Review of a patient's symptoms, activity, medications, concurrent medical problems, or possible toxic exposures can be useful in making a PD diagnosis. In addition, a patient may be tested for the presence or absence of genetic mutations that can indicate an increased likelihood of having or developing a neurodegenerative condition. For example, the presence of one or more specific mutations or polymorphisms in the NURR1, alpha-synuclein, parkin, MAPT, DJ-1, PINK1, SNCA, NAT2, or LRRK2 genes may be used to diagnose a patient as having or being at risk of having a neurodegenerative condition. See, e.g., U.S. Patent Application Publication Nos. 2003-0119026 and 2005-0186591; Bonifati, Minerva Med. 96:175-0.186, 2005; and Cookson et al., Curr. Opin. Neurol. 18:706-711, 2005, each of which is hereby incorporated by reference.

### Compounds of Formula I

As discussed herein, certain targeted TKI's have been found to be suitable candidates for the treatment of neurodegenerative conditions for their ability to inhibit certain tyrosine kinases such as PDGFR, src and c-kit and cross the blood-brain barrier. One class of such targeted TKI's includes the compounds disclosed in WO 2007/075869.

Targeted TKI's suitable for the presently disclosed methods and pharmaceutical compositions are compounds of Formula I:

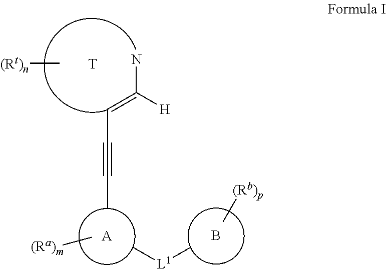

or a tautomer, or an individual isomer or a mixture of isomers thereof wherein:
Ring T is a 5-membered heteroaryl ring containing 1 or 2 nitrogens with the remaining ring atoms being carbon, substituted on at least two ring atoms with R^{t} groups, at least two of which being located on adjacent ring atoms, and, together with the atoms to which they are attached, forming a saturated, partially saturated or unsaturated 5- or 6-membered ring (Ring E), containing 0-3 heteroatoms selected from O, N, and S and being optionally substituted with 1-4 R^{e} groups;
Ring A is a 5- or 6-membered aryl or heteroaryl ring and is optionally substituted with 1-4 R^{a} groups;
Ring B is a 5- or 6-membered aryl or heteroaryl ring;
L¹ is selected from NR¹C(O), C(O)NR¹, NR¹C(O)O, NR¹C(O)NR¹, and OC(O)NR¹;
each occurrence of R^{a}, R^{b} and R^{t} is independently selected from the group consisting of halo, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;
R^{e}, at each occurrence, is independently selected from the group consisting of halo, ═O, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;

R¹, R² and R³ are independently selected from H, alkyl, alkenyl, alkynyl, cycloalkyl, cycloalkenyl, cycloalkynyl, aryl, heterocyclic and heteroaryl;

alternatively, R² and R³, taken together with the atom to which they are attached, form a 5- or 6-membered saturated, partially saturated or unsaturated ring, which can be optionally substituted and which contains 0-2 heteroatoms selected from N, O and S(O)ᵣ;
each occurrence of R⁴ is independently selected from alkyl, alkenyl, alkynyl, cycloalkyl, cycloalkenyl, cycloalkynyl, aryl, heterocyclic and heteroaryl;
each of the alkyl, alkenyl, alkynyl, cycloalkyl, cycloalkenyl, cycloalkynyl, aryl, heterocyclic and heteroaryl moieties is optionally substituted;
m is 0, 1, 2, 3 or 4;
n is 2 or 3;
p is 0, 1, 2, 3, 4 or 5; and,
r is 0, 1 or 2;

or a pharmaceutically acceptable salt, solvate or hydrate thereof.

The following portions of this section disclose various subgenuses of compounds of Formula I. In each subgenus, any variable not explicitly mentioned has the meaning defined by the genus immediately above, unless explicitly indicated otherwise.

In certain embodiments in the compound of Formula I, Ring T is:

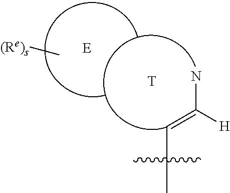

wherein Ring E is a 5- or 6-membered unsaturated ring comprising 0-3 heteroatoms selected from O, N, and S, and s is 0, 1, 2, 3 or 4.

Compounds useful for methods and pharmaceutical compositions disclosed herein include those in which Ring T has the following structure:

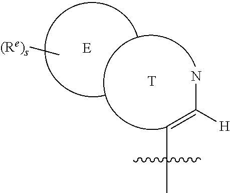

where Ring E is a 5- or 6-membered unsaturated ring (formed by two Rt groups together with the Ring T atoms to which they are attached, as described above) and s is 0, 1, 2, 3 or 4. These are illustrated by the compounds of Formula I in which the fused Ring T ring system is one of the following (in which one of the optional Re substituents is depicted):

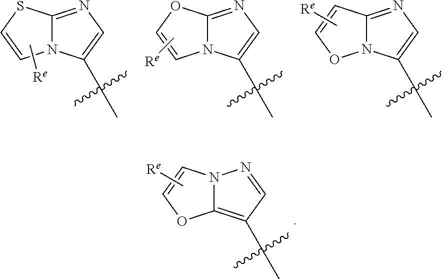

In certain embodiments in the compounds of Formula I, Ring T is a bicyclic heteroaryl ring selected from:

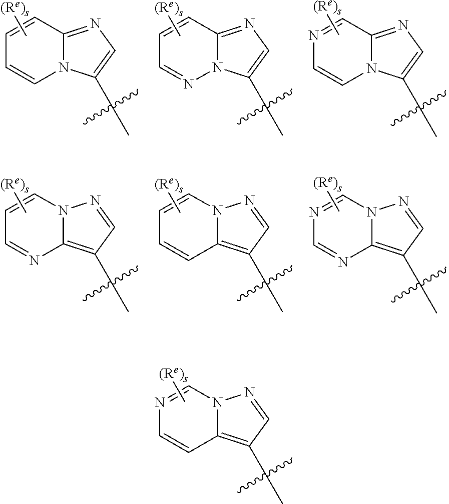

and s is 0, 1, 2, 3 or 4.

For the previously described class and subclasses of compounds, as in all compounds of this invention, Ring A and Ring B are as previously defined.

In certain of these embodiments, Ring A is selected from:

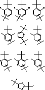

In certain embodiments in the compounds of Formula I, Ring B is a 5 or 6-membered aryl or heteroaryl ring as defined herein.

In certain of these embodiments. Ring B is:

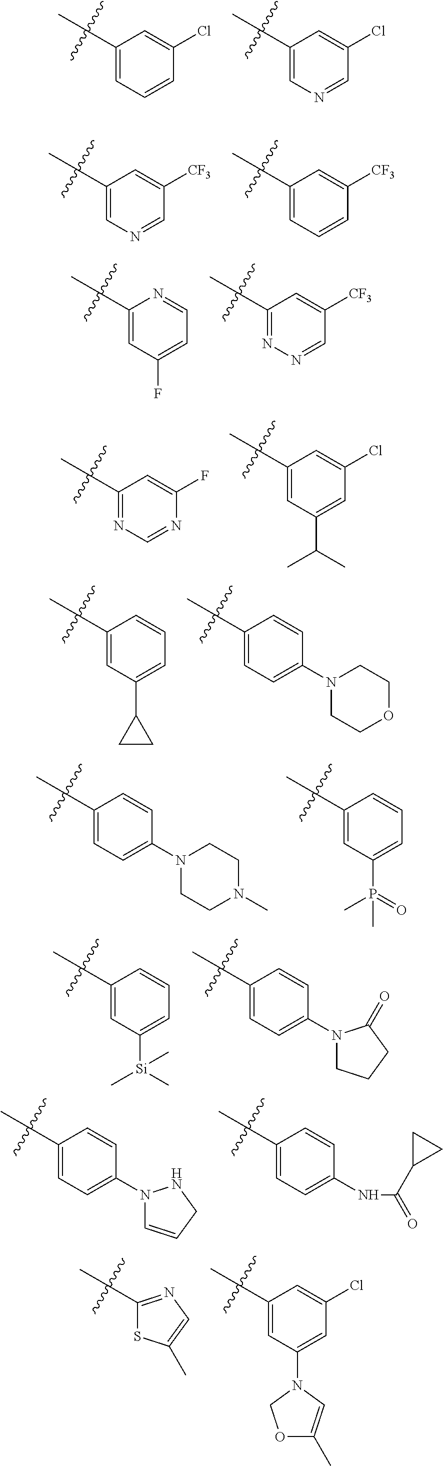

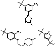

In certain embodiments in the compounds of Formula I, Rings A and B are aryl.

In certain embodiments in the compounds of Formula I, one of the R^{b} substituents is a 5- or 6-membered ring (Ring C), which may be heteroaryl or heterocyclic, comprising carbon atoms and 1-3 heteroatoms independently selected from O, N and S(O)ᵣ, and Ring C being optionally substituted on carbon or heteroatom(s) with 1 to 5 substituents R^{c}.

In certain embodiments, the targeted TKI is a compound of the Formula II:

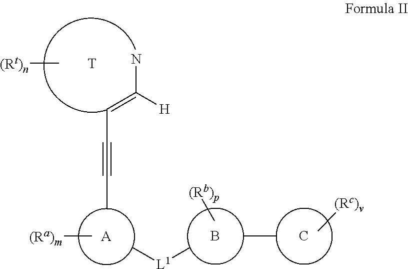

wherein:

Ring C is a 5- or 6-membered heterocyclic or heteroaryl ring, comprising carbon atoms and 1-3 heteroatoms independently selected from O, N and S(O)ᵣ;

R^{c}, at each occurrence, is independently selected from halo, ═O, —CN, —NO₂, —R⁴, —OR², —NR²R³, —C(O)YR², —OC(O)YR², —NR²C(O)YR², —Si(R²)₃, —SC(O)YR², —NR²C(═S)YR², —OC(═S)YR², —C(═S)YR², —YC(═NR³)YR², —YP(═O)(YR⁴)(YR⁴), —NR²SO₂R², —S(O)ᵣR², —SO₂NR²R³ and —NR²SO₂NR²R³, wherein each Y is independently a bond, —O—, —S— or —NR³—;

and,

v is 0, 1, 2, 3, 4 or 5.

In certain of these embodiments, Ring C is selected from the group consisting of:

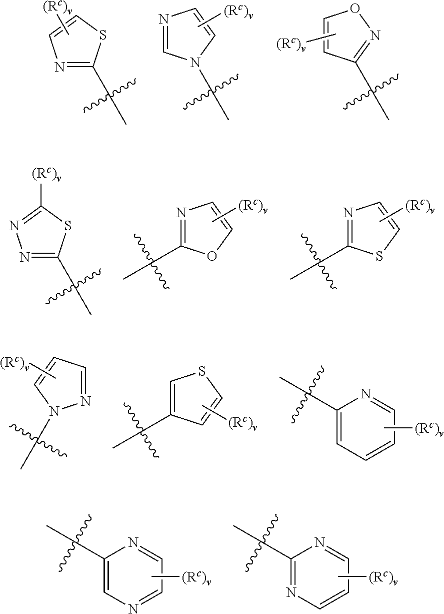

in which R^{c} and v are as defined above.

In certain embodiments in the compounds of Formula I where Ring C is present, Rings A and B are aryl.

In certain embodiments in the compound of Formula I where Ring C is present, Ring T is:

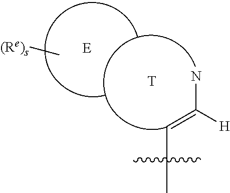

wherein Ring E is a 5- or 6-membered unsaturated ring comprising 0-3 heteroatoms selected from O, N, and S, and s is 0, 1, 2, 3 or 4.

Illustrative subsets of such compounds of Formula I include those having the following structures:

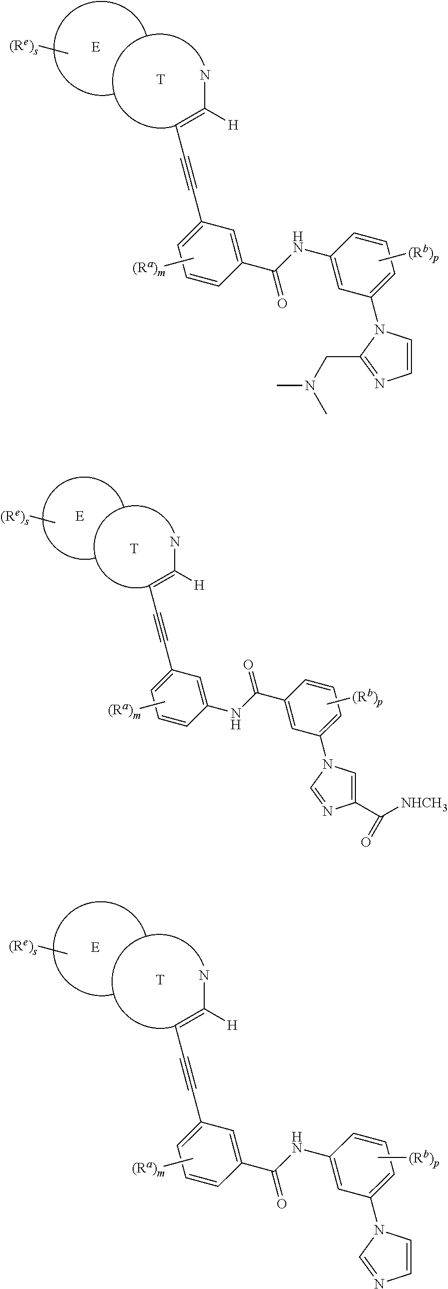

as embodied by the following non-limiting illustrative examples:

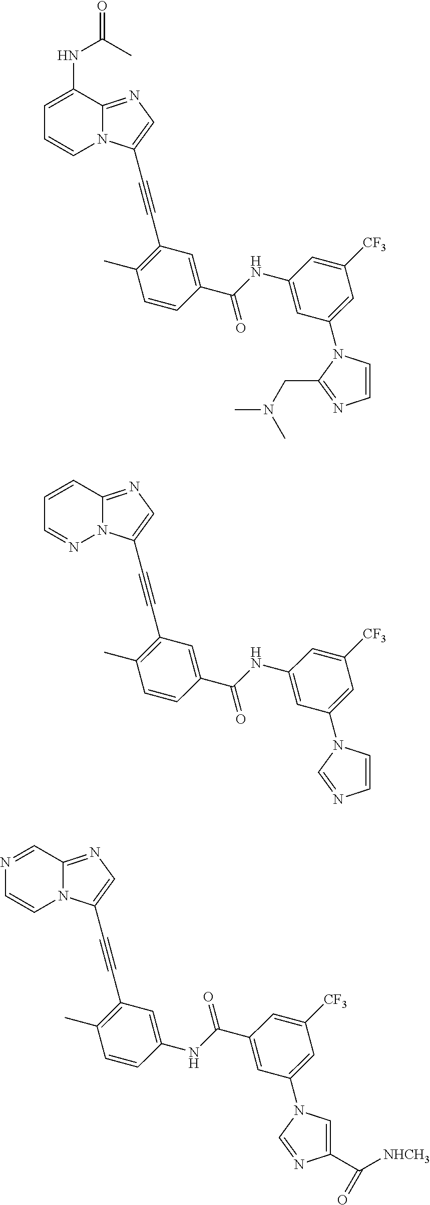 [SMILES: CC(=O)Nc1cccn2c(C#Cc3cc(C(=O)Nc4cc(-n5ccnc5CN(C)C)cc(C(F)(F)F)c4)ccc3C)cnc12 ; Cc1ccc(C(=O)Nc2cc(-n3ccnc3)cc(C(F)(F)F)c2)cc1C#Cc1cnc2cccnn12 ; CNC(=O)c1cn(-c2cc(C(=O)Nc3ccc(C)c(C#Cc4cnc5cnccn45)c3)cc(C(F)(F)F)c2)cn1]

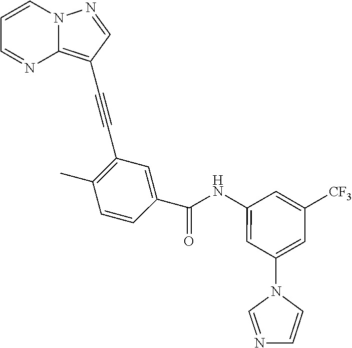 [SMILES: Cc1ccc(C(=O)Nc2cc(-n3ccnc3)cc(C(F)(F)F)c2)cc1C#Cc1cnn2cccnc12]

in which several illustrative -[Ring A]-[L¹]-[Ring B]-[Ring C]- portions are depicted.

In certain embodiments in the compounds of Formula I, Ring C is imidazolyl. Compounds of interest include among others, compounds of Formula II in which Ring C is an imidazole ring, optionally substituted with one or more R^{e} groups. Of particular interest, are compounds of this subclass in which Ring C bears a single lower alkyl (e.g., methyl) R^{c} group.

In certain of these embodiments where Ring C is imidazolyl, the targeted TKI is a compound selected from Formulae IIa, IIb, or IIc:

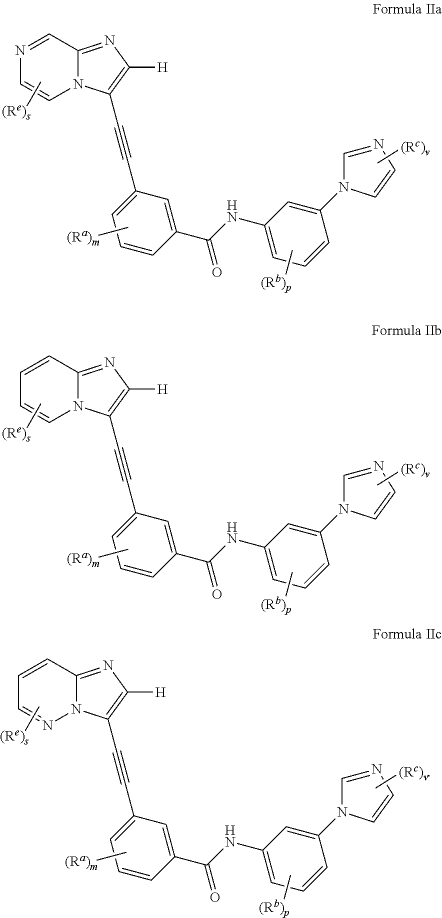

In certain embodiments within these embodiments, s is 0; m, p and v are 1; R^{a} and R^{c} are methyl; and R^{b} is CF₃.

In certain embodiments in the compounds of Formula I, the targeted TKI is a compound of the formula:

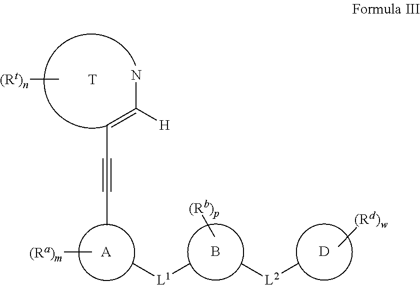

wherein:

Ring D represents a 5-, 6-heterocyclic or heteroaryl ring comprising carbon atoms and 1-3 heteroatoms independently selected from O, N and S(O)ᵣ;

L² is (CH₂)_{z}, O(CH₂)_{z}, NR³(CH₂)_{z}, S(CH₂)ₓ or (CH₂)ₓNR³C(O)(CH₂)ₓ in either direction;

R^{d}, at each occurrence, is selected from the group consisting of H, halo, ═O, —CN, —NO₂, —R⁴, —OR², —NR²R³, —C(O)YR², —OC(O)YR², —NR²C(O)YR², —SC(O)YR², NR²C(═S)YR², —OC(═S)YR², —C(═S)YR², —YC(═NR³)YR², —YP(═O)(YR⁴)(YR⁴), —Si(R²)₃, —NR²SO₂R², —S(O)ₙR², —SO₂NR²R³ and —NR²SO₂NR²R³, wherein each Y is independently a bond, —O—, —S— or —NR³—;

R¹, R² and R³ are independently selected from H, alkyl, alkenyl, alkynyl, cycloalkyl, cycloalkenyl, cycloalkynyl, aryl, heterocyclic and heteroaryl;

alternatively, R² and R³, taken together with the atom to which they are attached, form a 5- or 6-membered saturated, partially saturated or unsaturated ring, which can be optionally substituted and which contains 0-2 heteroatoms selected from N, O and S(O)ᵣ;

each occurrence of R⁴ is independently selected from alkyl, alkenyl, alkynyl, cycloalkyl, cycloalkenyl, cycloalkynyl, aryl, heterocyclic and heteroaryl;

each of the alkyl, alkenyl, alkynyl, cycloalkyl, cycloalkenyl, cycloalkynyl, aryl, heterocyclic and heteroaryl moieties is optionally substituted;

p is 0, 1, 2, 3 or 4;

w is 0, 1, 2, 3, 4 or 5;

x is 0, 1, 2 or 3; and,

z is 1, 2, 3 or 4.

In certain of these embodiments where Ring D is present, Ring T has the following structure:

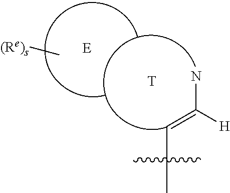

wherein Ring E is a 5- or 6-membered unsaturated ring comprising 0-3 heteroatoms selected from O, N, and S, and s is 0, 1, 2, 3 or 4.

Non-limiting examples of such compounds include those having the following structures:

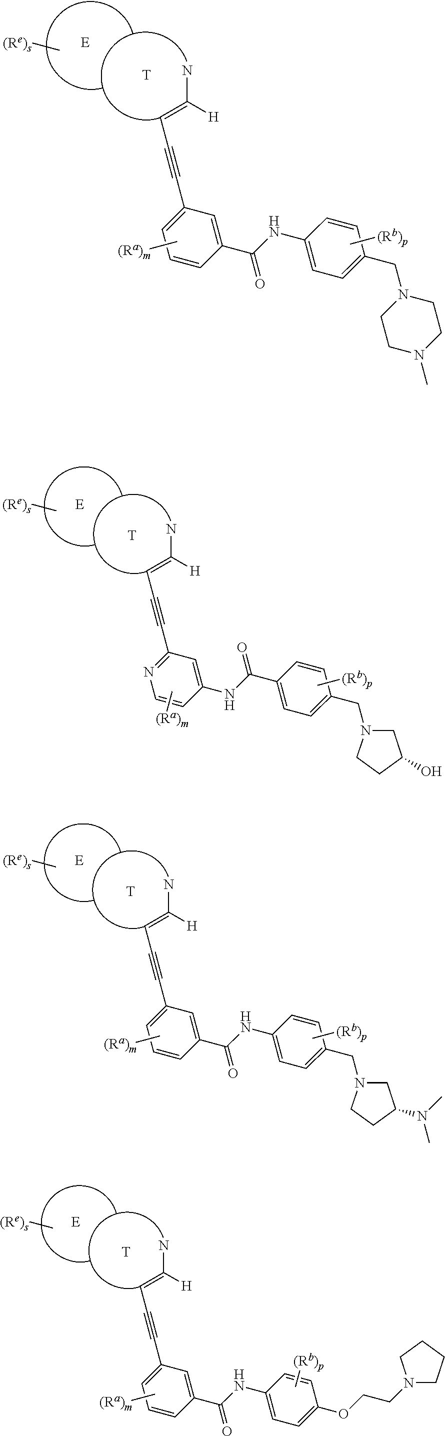

as illustrated by the following examples:

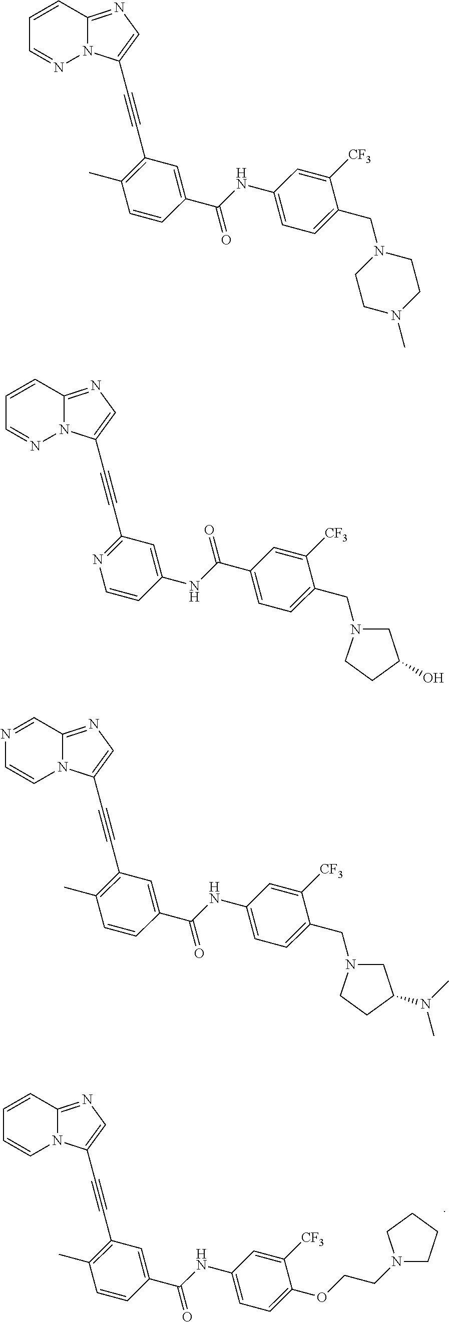 [SMILES: Cc1ccc(C(=O)Nc2ccc(CN3CCN(C)CC3)c(C(F)(F)F)c2)cc1C#Cc1cnc2cccnn12 ; O=C(Nc1ccnc(C#Cc2cnc3cccnn23)c1)c1ccc(CN2CC[C@@H](O)C2)c(C(F)(F)F)c1 ; Cc1ccc(C(=O)Nc2ccc(CN3CC[C@@H](N(C)C)C3)c(C(F)(F)F)c2)cc1C#Cc1cnc2cnccn12 ; Cc1ccc(C(=O)Nc2ccc(OCCN3CCCC3)c(C(F)(F)F)c2)cc1C#Cc1cnc2ccccn12]

In certain of these embodiments where Ring D is present, Rings A and B are aryl.

In certain of these embodiments where Ring D is present, Ring T is a bicyclic heteroaryl ring selected from:

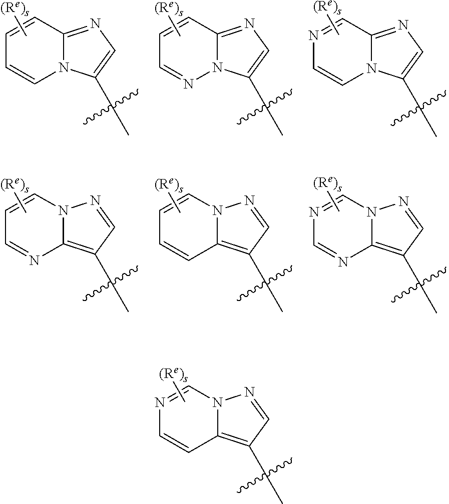

and s is 0, 1, 2, 3 or 4.

Non-limiting, illustrative examples of -[Ring B]-[L²]-[Ring D] moieties in compounds of Formula III include among others:

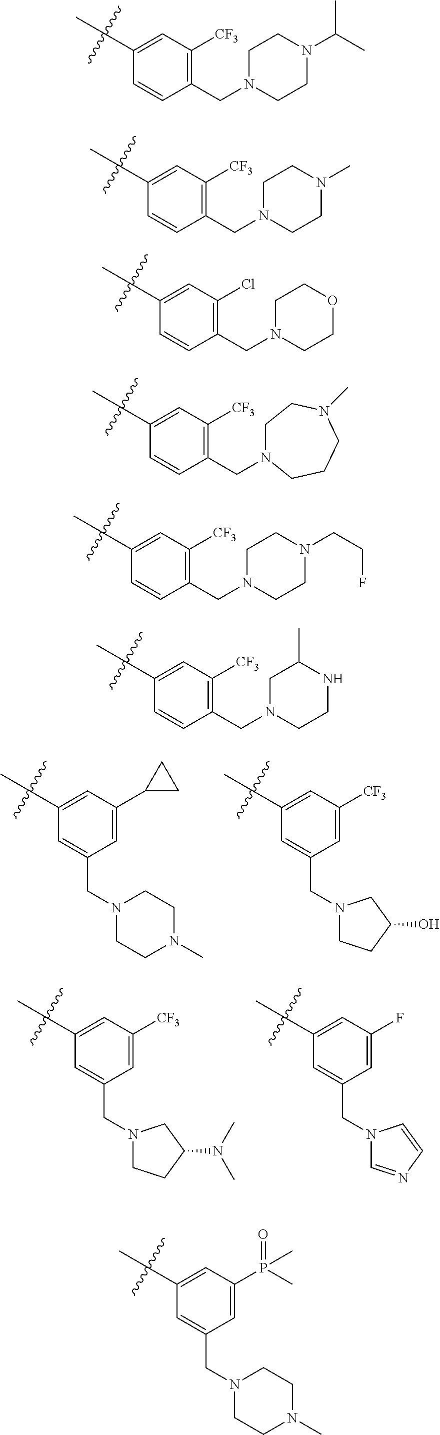

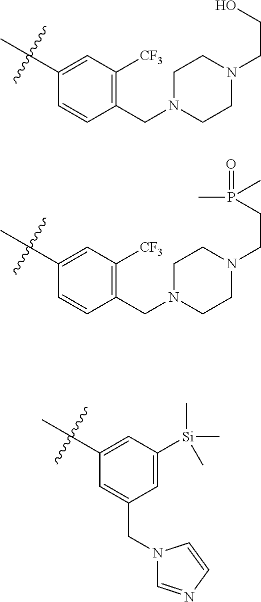 [SMILES: *c1ccc(CN2CCN(CCO)CC2)c(C(F)(F)F)c1 ; *c1ccc(CN2CCN(CCP(C)(C)=O)CC2)c(C(F)(F)F)c1 ; *c1cc(Cn2ccnc2)cc([Si](C)(C)C)c1]

In certain embodiments in the compounds of Formula I, compounds of interest include among others, compounds of Formula III in which Ring D is a piperazine ring, substituted on nitrogen with R^{d}. Of particular current interest, are compounds of this subclass in which R^{d} is a substituted or unsubstituted lower (i.e., 1-6 carbon) alkyl as illustrated by N-methylpiperazine moieties in some of the following examples.

In certain of these embodiments where Ring D is present, Ring D is piperazinyl and L² is CH₂. In certain of these embodiments, the targeted TKI is a compound selected from Formulae IIIa, IIIb, and IIIc:

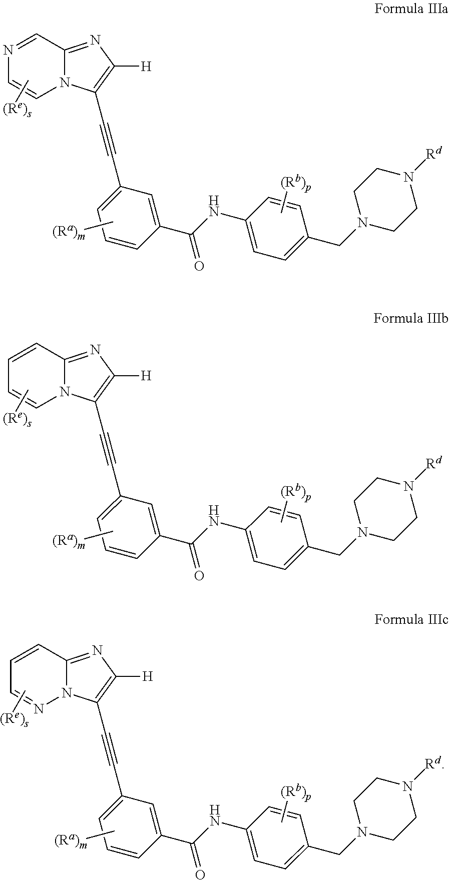

In certain embodiments within these embodiments, s is 0, m is 1, p is 1, R^{a} is methyl, R^{b} is CF₃, and R^{d} is methyl or —CH₂CH₂OH.

In certain embodiments in the compounds of Formula II and Formula III, Ring T is any 6/5 fused heteroaryl ring system, optionally substituted with up to three R^{e} groups. Of particular interest are compounds in which s is 0. Also of interest are those in which s is 1-3 and at least one R^{e} is halo, lower alkyl, alkoxy, amino, —NH-alkyl, —C(O)NH-alkyl, —NHC(O)-alkyl, —NHC(O)NH-alkyl, —NHC(NH)-alkyl, —NHC(NH)NH₂, —NH(CH₂)ₓ-heteroaryl, —NH(CH₂)ₓ-heterocycle, —NH(CH₂)ₓ-aryl or —(CH₂)ₓC(O)NH₂, in which x is 0, 1, 2 or 3 and “alkyl” includes straight (i.e., unbranched and acyclic), branched and cyclic alkyl groups and in which aryl, heteroaryl, heterocyclyl rings are optionally substituted. Illustrative, non-limiting, examples of the foregoing include compounds of Formulas II and III in which Ring T is one of the following:

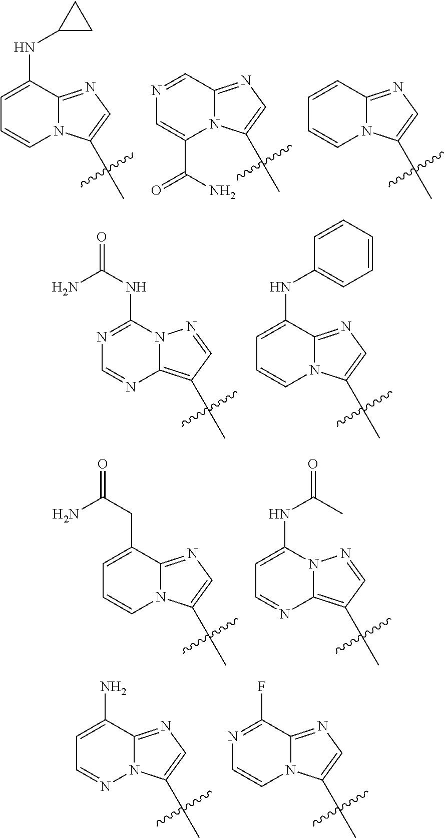

In certain embodiments in the compounds of Formula II and Formula III, Ring T is an optionally substituted imidazo[1,2-a]pyridine, imidazo[1,2-b]pyridazine, imidazo[1,2-a]pyrazine, pyrazolo[1,5-a]pyrimidine, pyrazolo[1,5-a]pyridine, pyrazolo[1,5-a]pyrimidine, and pyrazolo[1,5-a][1,3,5]triazine.

In certain of these embodiments in the compounds of Formula II and Formula III, Rings A and B are aryl.

Illustrative, non-limiting examples of this subclass include compounds of Formulas IIa, IIb, IIc, IIIa, IIIb and IIIc:

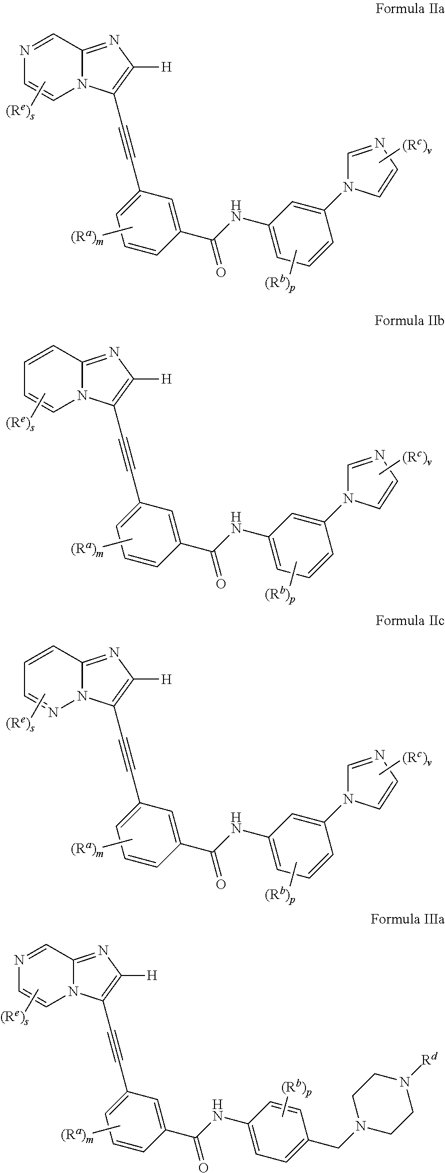

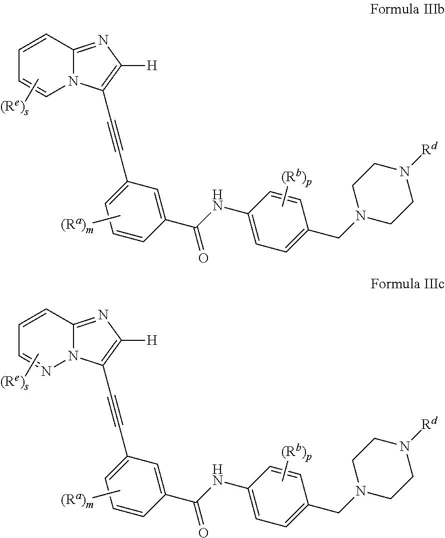

in which the variables, e.g., R^{a}, R^{b}, R^{c}, R^{d}, R^{e}, m and p, are as previously defined and s is an integer from 0 to 4.

In certain embodiments in the compounds of Formulas IIa, IIb and IIc, s is 0; m, p and v are 1; and, R^{a} is CH₃, R^{b} is CF₃ and R^{c} is methyl.

In certain embodiments in the compounds of Formulas IIIa, IIIb, IIIc, s is 0; m and p are 1; and, R^{a} is CH₃, R^{b} is CF₃ and R^{d} is CH₃ or CH₂CH₂OH.

In certain embodiments, the targeted TKI is a compound selected from the group consisting of:
N-(3-(1H-imidazol-1-yl)-5-(trifluoromethyl)phenyl)-3-(imidazo[1,2-a]pyrazin-3-ylethynyl)-4-methylbenzamide;
3-(Imidazo[1,2-a]pyrazin-3-ylethynyl)-4-methyl-N-(4-((4-methylpiperazin-1-yl)methyl)-3-(trifluoromethyl)phenyl)benzamide;
N-(3-(2-((dimethylamino)methyl)-1H-imidazol-1-yl)-5-(trifluoromethyl)phenyl)-3-(imidazo[1,2-a]pyrazin-3-ylethynyl)-4-methylbenzamide;
3-(Imidazo[1,2-a]pyridin-3-ylethynyl)-4-methyl-N-(3-(4-methyl-1H-imidazol-1-yl)-5-(trifluoromethyl)phenyl)benzamide;
N-(3-(1H-imidazol-1-yl)-5-(trifluoromethyl)phenyl)-3-(imidazo[1,2-a]pyridin-3-ylethynyl)-4-methylbenzamide;
3-(Imidazo[1,2-a]pyridin-3-ylethynyl)-4-methyl-N-(4-(trifluoromethyl)pyridin-2-yl)benzamide;
N-(5-tert-butylisoxazol-3-yl)-3-(imidazo[1,2-a]pyridin-3-ylethynyl)-4-methylbenzamide;
3-(Imidazo[1,2-a]pyridin-3-ylethynyl)-4-methyl-N-(4-((4-methylpiperazin-1-yl)methyl)-3-(trifluoromethyl)phenyl)benzamide;
N-(3-(2-((dimethylamino)methyl)-1H-imidazol-1-yl)-5-(trifluoromethyl)phenyl)-3-(imidazo[1,2-a]pyridin-3-ylethynyl)-4-methylbenzamide;
3-((8-Acetamidoimidazo[1,2-a]pyridin-3-yl)ethynyl)-4-methyl-N-(4-(trifluoromethyl)pyridin-2-yl)benzamide;
N-(3-(1H-imidazol-1-yl)-5-(trifluoromethyl)phenyl)-3-((8-acetamidoimidazo[1,2-a]pyridin-3-yl)ethynyl)-4-methylbenzamide;
4-Methyl-3-((8-(4-(methylsulfonyl)phenylamino)imidazo[1,2-a]pyridin-3-yl)ethynyl)-N-(4-(trifluoromethyl)pyridin-2-yl)benzamide;
4-methyl-3-((8-(4-sulfamoylphenylamino)imidazo[1,2-a]pyridin-3-yl)ethynyl)-N-(4-(trifluoromethyl)pyridin-2-yl)benzamide;
(R)—N-(4-((3-(Dimethylamino)pyrrolidin-1-yl)methyl)-3-(trifluoromethyl)phenyl)-3-(imidazo[1,2-b]pyridazin-3-ylethynyl)-4-methylbenzamide;
N-(3-(Imidazo[1,2-b]pyridazin-3-ylethynyl)-4-methylphenyl)-4-((4-methylpiperazin-1-yl)methyl)-3-(trifluoromethyl)benzamide;
3-(Imidazo[1,2-b]pyridazin-3-ylethynyl)-4-methyl-N-(4-((4-methylpiperazin-1-yl)methyl)-3-(trifluoromethyl)phenyl)benzamide;
N-(3-Chloro-4-((4-methylpiperazin-1-yl)methyl)phenyl)-3-(imidazo[1,2-b]pyridazin-3-ylethynyl)-4-methylbenzamide;
N-(3-Cyclopropyl-4-((4-methylpiperazin-1-yl)methyl)phenyl)-3-(imidazo[1,2-b]pyridazin-3-ylethynyl)-4-methylbenzamide;
3-(Imidazo[1,2-b]pyridazin-3-ylethynyl)-N-(4-((4-methylpiperazin-1-yl)methyl)-3-(trifluoromethyl)phenyl)benzamide;
N-(4-((4-(2-Hydroxyethyl)piperazin-1-yl)methyl)-3-(trifluoromethyl)phenyl)-3-(imidazo[1,2-b]pyridazin-3-ylethynyl)-4-methylbenzamide; and
3-(Imidazo[1,2-b]pyridazin-3-ylethynyl)-4-methyl-N-(4-(piperazin-1-ylmethyl)-3-(trifluoromethyl)phenyl)benzamide,
   or a pharmaceutically acceptable salt thereof.

A targeted TKI of particular interest that is useful for the presently disclosed methods and pharmaceutical compositions is 3-(Imidazo[1,2-b]pyridazin-3-ylethynyl)-4-methyl-N-(4-((4-methylpiperazin-1-yl)methyl)-3-(trifluoromethyl)phenyl)benzamide or a pharmaceutically acceptable salt thereof. A pharmaceutically acceptable salt of particular interest for this compound (ponatinib) is its hydrochloride salt.

In certain embodiments in the compounds of Formula I, the targeted tyrosine kinase inhibitor is a compound of the formula:

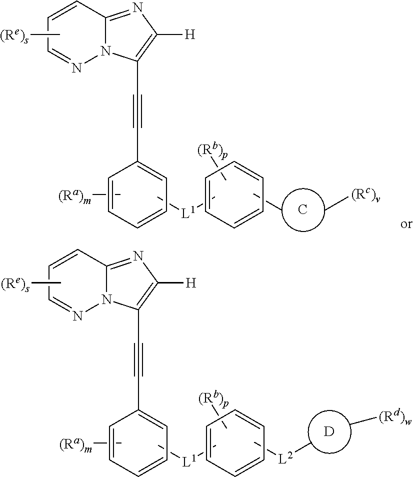

wherein:

L¹ is NR¹C(O) or C(O)NR¹;

Ring D is a 5- or 6-membered heterocyclyl or heteroaryl ring comprising carbon atoms and 1-3 heteroatoms independently selected from O, N, and S(O)ᵣ;

Ring C is a 5- or 6-membered heterocyclyl or heteroaryl ring, comprising carbon atoms and 1-3 heteroatoms independently selected from O, N, and S(O)ᵣ;

L² is —(CH₂)_{z}—;

each occurrence of R^{a} is independently selected from the group consisting of halo, alkyl, and cycloalkyl;

each occurrence of R^{b} is independently selected from the group consisting of halo, alkyl, and cycloalkyl;

each occurrence of R^{c} is independently selected from the group consisting of halo, alkyl, and cycloalkyl;

each occurrence of R^{d} is independently selected from the group consisting of halo, alkyl, cycloalkyl, and —NR²R³;

each occurrence of R^{e} is independently selected from the group consisting of halo, alkyl, cycloalkyl, —NR²R³, alkoxy, amino, —NH-alkyl, —C(O)NH-alkyl, —NHC(O)-alkyl, —NHC(O)NH-alkyl, —NHC(NH)-alkyl, —NHC(NH)NH₂, —NH(CH₂)ₓ-heteroaryl, —NH(CH₂)ₓ-heterocyclyl, —NH(CH₂)ₓ-aryl, and —(CH₂)ₓC(O)NH₂, wherein x is 0, 1, 2 or 3;

each of R¹, R² and R³ is independently selected from H, alkyl, alkenyl, alkynyl, cycloalkyl, cycloalkenyl, cycloalkynyl, aryl, heterocyclyl, and heteroaryl, or R² and R³, taken together with the nitrogen atom to which at least one of R² and R³ is attached, form a 5- or 6-membered heterocyclyl or heteroaryl ring;

each of the alkyl, alkenyl, alkynyl, cycloalkyl, cycloalkenyl, cycloalkynyl, and heterocyclyl moieties is unsubstituted or substituted with one or more groups selected from amino, alkylamino, dialkylamino, aminocarbonyl, halogen, alkyl, alkenyl, alkynyl, aryl, heteroaryl, carbocyclyl, heterocyclyl, alkylaminocarbonyl, dialkylaminocarbonyl, alkylaminocarbonyloxy, dialkylaminocarbonyloxy, nitro, cyano, carboxy, alkoxycarbonyl, alkylcarbonyl, hydroxy, alkoxy, acyloxy, haloalkoxy, ═O, ═S, ═NH, ═NNR²R³, ═NNHC(O)R², ═NNHCO₂R², and ═NNHSO₂R², and each of the aryl and heteroaryl moieties is unsubstituted or substituted with one or more groups selected from amino, alkylamino, dialkylamino, aminocarbonyl, halogen, alkyl, alkenyl, alkynyl, aryl, heteroaryl, carbocyclyl, heterocyclyl, alkylaminocarbonyl, dialkylaminocarbonyl, alkylaminocarbonyloxy, dialkylaminocarbonyloxy, nitro, cyano, carboxy, alkoxycarbonyl, alkylcarbonyl, hydroxy, alkoxy, acyloxy, and haloalkoxy;

m is 0, 1, 2, 3, or 4;

p is 0, 1, 2, 3, or 4;

r is 0, 1, or 2;

s is 0, 1, 2, or 3;

v is 0, 1, 2, 3, 4, or 5;

w is 0, 1, 2, 3, 4, or 5; and

z is 1, 2, 3 or 4;

or a pharmaceutically acceptable salt thereof.

### Formulations, Dosage and Administration

Compounds of Formula I can be formulated into a pharmaceutical composition that comprises a compound of Formula I (as an active pharmaceutical ingredient) or a pharmaceutically acceptable salt thereof and a pharmaceutically acceptable carrier. Similarly, ponatinib, or a pharmaceutically acceptable salt thereof, such as the mono HCl salt, can be formulated for administration, such as oral administration, using any of the materials and methods useful for such purposes.

Pharmaceutically acceptable compositions containing a compound of Formula I suitable for administration may be formulated using conventional materials and methods, a wide variety of which are well known. While the composition may be in solution, suspension or emulsion form, solid oral dosage forms such as capsules, tablets, gel caps, caplets, etc. are of particular interest for the treatment of PD. Methods well known in the art for making formulations, including the foregoing unit dosage forms, are found, for example, in “Remington: The Science and Practice of Pharmacy” (20th ed., ed. A. R. Gennaro, 2000, Lippincott Williams & Wilkins). A compound of Formula I such as ponatinib (or a pharmaceutically acceptable salt thereof) may be provided neat in capsules, or combined with one or more optional, pharmaceutically acceptable excipients such as fillers, binders, stabilizers, preservatives, glidants, disintegrants, colorants, film coating, etc., as illustrated below.

For example, white opaque capsules were prepared containing nominally 2 mg of ponatinib free base, provided as the hydrochloride salt, with no excipients. White opaque capsules were also prepared containing 5 mg, 15 mg, or 20 mg of ponatinib free base, provided as the hydrochloride salt, mixed with conventional excipients. Inactive ingredients used as excipients in an illustrative capsule blend include one or more of a filler, a flow enhancer, a lubricant, and a disintegrant. For instance, a capsule blend was prepared for the 5, 15 and 20 mg capsules, containing the ponatinib HCl salt plus colloidal silicon dioxide (ca. 0.3% w/w, a flow enhancer), lactose anhydrous (ca. 44.6% w/w, a filler), magnesium stearate (ca. 0.5% w/w, a lubricant), microcrystalline cellulose (ca. 44.6% w/w, a filler), and sodium starch glycolate (ca. 5% w/w, a disintegrant). The capsule shell contains gelatin and titanium dioxide.

The formulation process used conventional blending and encapsulation processes and machinery. The hydrochloride salt of ponatinib and all blend excipients except magnesium stearate were mixed in a V-blender and milled through a screening mill. Magnesium stearate was added and the material was mixed again. The V-blender was sampled to determine blend uniformity. The blend was tested for bulk density, tap density, flow, and particle size distribution. The blend was then encapsulated into size “3”, size “4”, or size “1” capsule shells, depending upon the strength of the unit dosage form.

Ponatinib was also formulated into tablets using conventional pharmaceutical excipients, including one or more of a filler or a mixture of fillers, a disintegrant, a glidant, a lubricant, a film coating, and a coating solvent in a blend similar to that used in the higher strength capsules. For example, tablets may be prepared using the following relative amounts and proportions (weight/weight): ponatinib (90 g provided as the HCl salt, 15.0% w/w), colloidal silicon dioxide (1.2 g, 0.2% w/w), lactose monohydrate (240.9 g, 40.15% w/w), magnesium stearate (3 g, 0.5% w/w), microcrystalline cellulose (240.9 g, 40.15% w/w), and sodium starch glycolate (24 g, 4.0% w/w), with the amount of lactose monohydrate adjusted based on the amount of drug used.

Ponatinib and the excipients may be mixed using the same sort of machinery and operations as was used in the case of capsules. The resultant, uniform blend may then be compressed into tablets by conventional means, such as a rotary tablet press adjusted for target tablet weight, e.g. 300 mg for 45 mg tablets or 100 mg for 15 mg tablets; average hardness of e.g., 13 kp for 45 mg tablets and 3 kp for 15 mg tablets; and friability no more than 1%. The tablet cores so produced may be sprayed with a conventional film coating material, e.g., an aqueous suspension of Opadry® II White, yielding for example a ˜2.5% weight gain relative to the tablet core weight.

After formulation with an appropriate pharmaceutically acceptable carrier in a desired dosage, the compositions of disclosed herein can be administered to humans and other animals orally, rectally, parenterally, intracisternally, intravaginally, intraperitoneally, topically (as by transdermal patch, powders, ointments, or drops), sublingually, bucally, as an oral or nasal spray, or the like.

In accordance with the methods, kits, and pharmaceutical compositions of the invention, a treatment will typically consist of a plurality of doses of a compound of Formula I that is administered over a period of time. Administration may be one or multiple times daily, weekly (or at some other multiple day interval) or on an intermittent schedule, with that cycle repeated a given number of times (e.g., 2-10 cycles) or indefinitely.

Optimal dosing will depend in part on the route of administration. Effective doses may be calculated according to the body weight or body surface area. Optimization of the appropriate dosages can readily be made by one skilled in the art in light of pharmacokinetic data observed in human clinical trials. The final dosage regimen will be determined by the attending physician, considering various factors which modify the action of the drugs, e.g., the drug's specific activity, the severity of the damage and the responsiveness of the subject, the age, condition, body weight, sex and diet of the subject, and other clinical factors.

In certain embodiments, a compound of Formula I is administered at a unit dose of 5-80 mg (e.g., from 5 to 10 mg, 10 to 25 mg, 25 to 35 mg, 35 to 50 mg, 50 to 60 mg, or 60 to 80 mg). In certain of these embodiments, the unit dose is 5-45 mg or 15-45 mg. Preferred dosage strengths for ponatinib include, but are not limited to 15 mg, 30 mg, and 45 mg.

Oral administration is of particular interest in the practice of the various embodiments of this invention, including oral administration on a daily schedule or on an intermittent schedule as mentioned above and at the dose levels mentioned above. By way of non-limiting example, daily oral administration of 5-80 mg of ponatinib, and in some cases, 5-45 mg of ponatinib, are of particular current interest. In certain embodiments, 5, 10, 15, 30 or 45 mg of ponatinib, e.g., ponatinib hydrochloride, are administered orally on a daily basis.

The amount and dosing schedule for ponatinib administered in any of the embodiments of the invention may be chosen or adjusted to produce a mean steady state trough concentration for ponatinib in plasma of from 5 to 200 nM (e.g., a mean steady state trough concentration for ponatinib of 5±2 nM, 8±3 nM, 12±3 nM, 15±3 nM, 20±5 nM, 30±5 nM, 40±5 nM, 50±10 nM, 60±10 nM, 80±20 nM, 100±20 nM, 120±20 nM, 150±25 nM, 175±25 nM, or 200±25 nM).

The amount and dosing schedule for ponatinib administered in any of the embodiments of the invention may be chosen or adjusted to be effective to measurably reduce the desired relevant kinase activity and/or β-amyloid in the brain of the subject.

In certain embodiments, the compound of Formula I is administered to the subject at an average daily dose of 3±1 mg, 5±2 mg, 8±2 mg, 12±3 mg, 15±3 mg, 20±4 mg, 25±5 mg, 30±6 mg, 40±8 mg, 45±9 mg, 50±10 mg, or 55±11 mg.

In certain embodiments, the compound of Formula I is administered to the subject on one or more days per week, including in some cases every day, every other day, every third day as well as schedules, such as, e.g., QD×6, QD×5 QD×4 QD×3 and QD×2 (i.e., 6, 5, 4, 3 or 2 days per week, respectively). On a given day, the drug may be given in one dose or may be divided into two or three doses administered during the course of the day (i.e., qd, bid or tidy.

Because compounds of Formula I are orally bioavailable, a compound of Formula I such as ponatinib may be given orally as well as parenterally (e.g., i.v.) or by other pharmaceutically acceptable routes of administration. Thus, the active compounds of the disclosure may be formulated for oral, buccal, intranasal, parenteral (e.g., intravenous, intramuscular or subcutaneous), rectal administration, in a form suitable for administration by inhalation or insufflation, or the active compounds may be formulated for topical administration.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

For buccal administration, the composition may take the form of tablets or lozenges formulated in conventional manner.

For intranasal administration or administration by inhalation, the active compounds of the disclosure are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the disclosure and a suitable powder base such as lactose or starch.

The active compounds of the disclosure may be formulated for parenteral administration by injection, including using conventional catheterization techniques or infusion. Routes of parenteral administration also include intravenous, intramuscular and subcutaneous. Formulations for injection may be presented in unit dosage form, e.g., in ampules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g., sterile pyrogen-free water, before use.

The active compounds of the disclosure may also be formulated in rectal compositions such as suppositories or retention enemas, e.g., containing conventional suppository bases such as cocoa butter or other glycerides.

For topical administration, a presently disclosed compound may be formulated as an ointment or cream.

Suitable modes of administration also include, but are not limited to, transdermal, vaginal, and ophthalmic.

### Synthesis of Compounds of Formula I

The synthesis of compounds of Formula I have been reported in WO 2007/075,869. For the convenience of the reader, the synthetic scheme is reproduced immediately below.

A compound of the present invention could be prepared as outlined in Scheme I to Scheme XIX and via standard methods known to those skilled in the art.

A palladium catalyzed Sonogashira coupling reaction is used to link the ‘top’ Ring T to the ‘bottom’ [Ring A]-[L¹]-[Ring B] moiety as illustrated in Scheme I and II. In Scheme I the Sonogashira coupling reaction is performed with an acetylenic ‘top’ Ring T and a ‘bottom’ [Ring A]-[L¹]-[Ring B] moiety which has been activated by the presence of a reactive group, W, which is an I, a Br or another reactive group permitting the desired coupling reaction. The variables in the W-[Ring A]-[L¹]-[Ring B] are as defined previously, Rings A and B being substituted with permitted R^{a} and R^{b} groups, respectively.

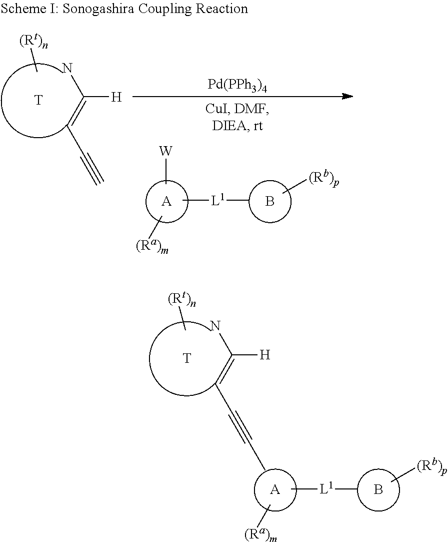

An alternative coupling reaction is described in Scheme II, in which Ring T is “activated” by the presence of a reactive group W (such as I or Br) and is coupled to the ‘bottom’ acetylenic [RingA]-L¹-[RingB] under similar Palladium catalyzed coupling conditions.

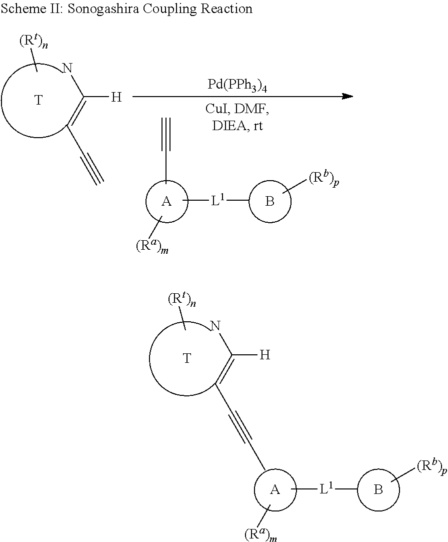

The Sonogashira coupling conditions described in Scheme I and II are applicable to all bicyclic heteroaryl Ring T's and useful to synthesize the compounds disclosed herein.

Several illustrative overall synthetic approaches to the preparation of the acetylenic Ring T moieties, based on known transformations, are illustrated below in Schemes III to VIII:

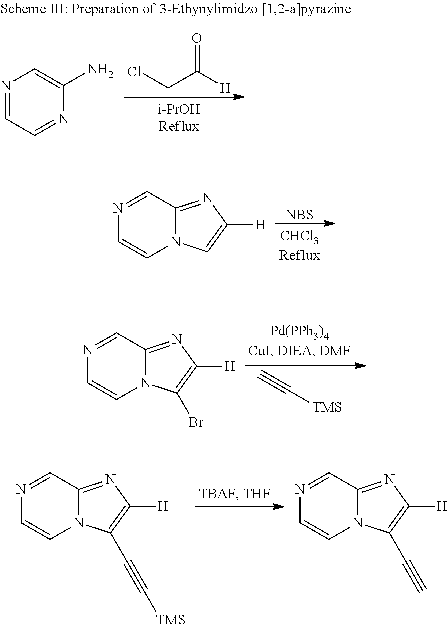

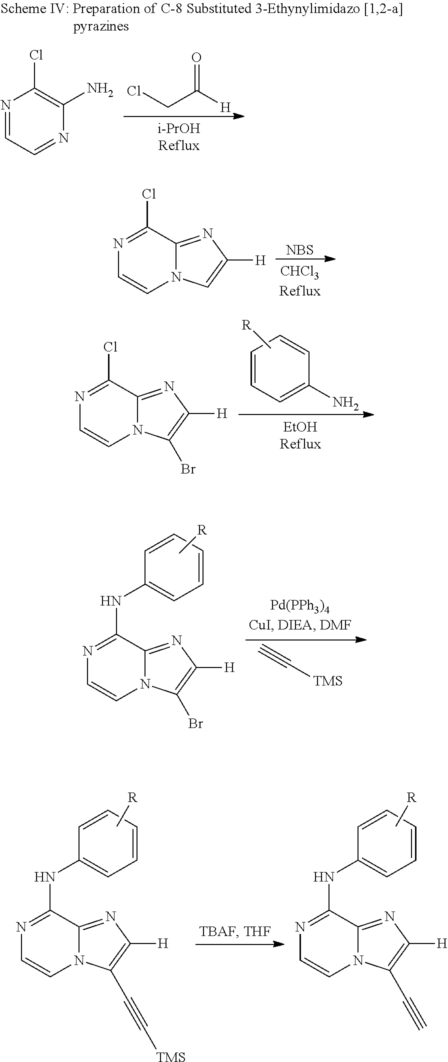

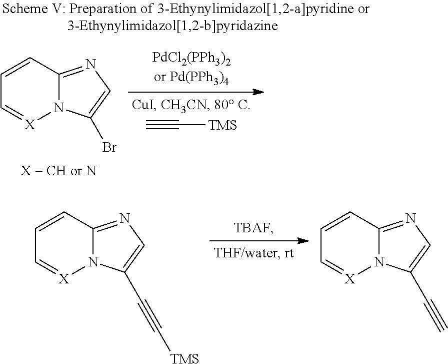

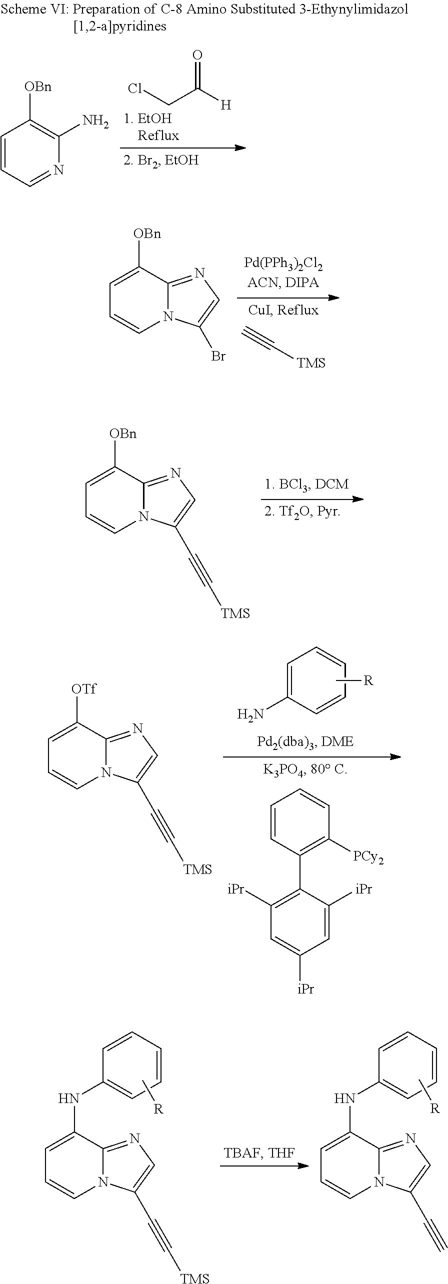

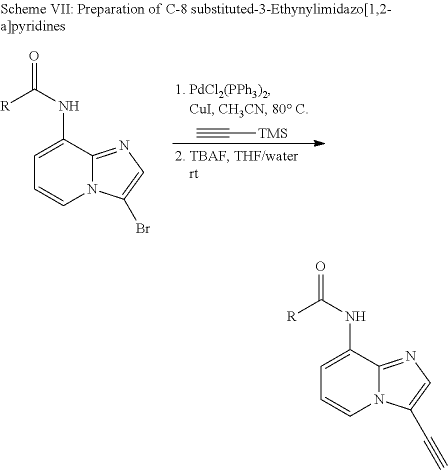

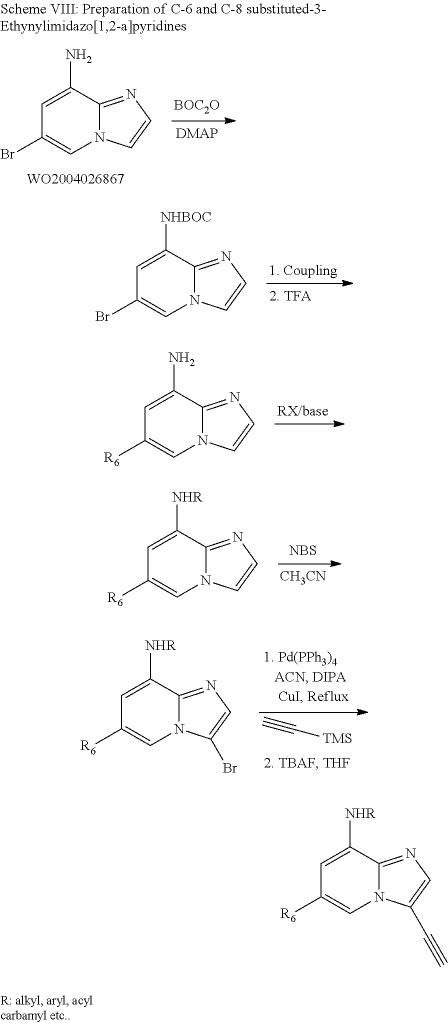

For the coupling step, see Malleron, J-L., Fiaud, J-C., Legros, J-Y. Handbook of Palladium Catalyzed Organic Reactions. San Diego: academic Press, 1997.

As one of ordinary skill in the art would recognize, these methods for the preparation of various substituted acetylenic Ring T groups, are widely applicable to various other fused bicyclic ring systems not shown.

Schemes IX to XIII below depict the synthesis of compounds of the formula W-[Ring A]-[L′]-[Ring B] which are useful as intermediates in the coupling reaction described in Schemes I and II.

It should be apparent that intermediates of the formula:

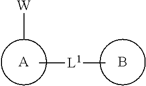

are of particular interest as their coupling reaction with the ‘top’ heteroaryl rings produces compounds of the present invention. The variable groups A, L′ and B are as previously defined and are optionally substituted as described herein, and W is I or an alternative reactive group permitting the desired coupling reaction.

Illustrative such intermediates include among others those of those following structures:

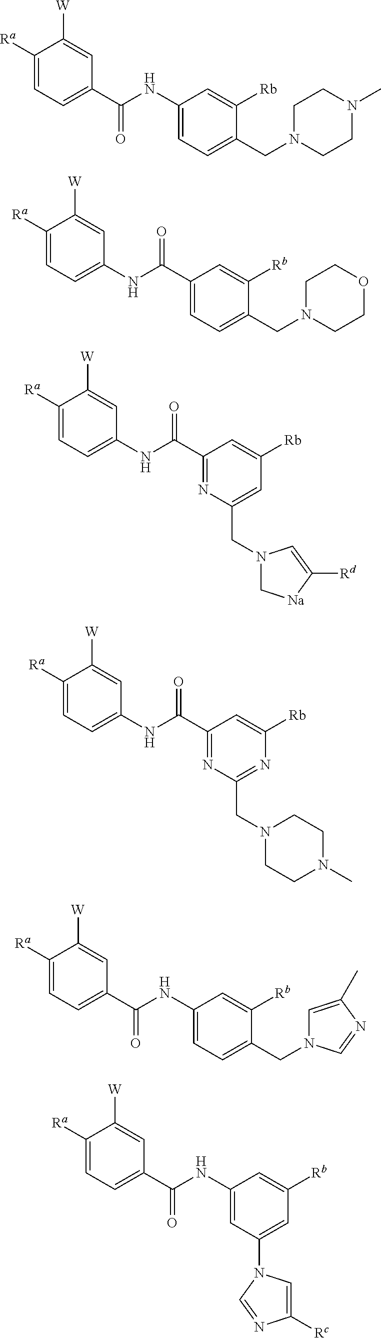

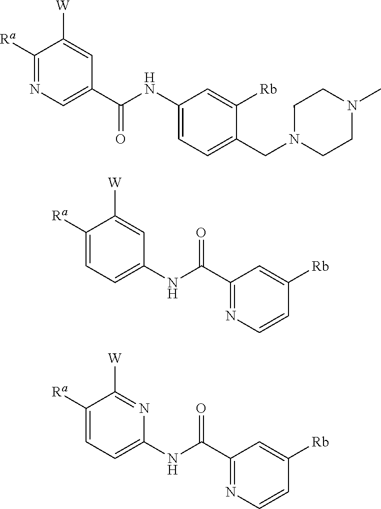

wherein the variables, e.g., R^{a}, R^{b}, R^{c} and R^{d}, are as previously defined. For instance, R^{a} in some embodiments is chosen from F or alkyl, e.g., Me, among others, and Rb in some embodiments is chosen from Cl, F, Me, t-butyl, —CF3 or —OCF3 among others. Those and other compounds of the formula W-[Ring A]-[L¹]-[Ring B] with the various permitted substituents are useful for preparing the corresponding compounds of the invention as are defined in the various formulae, classes and subclasses disclosed herein.

Some illustrative synthetic routes for the preparation of reagents and representative intermediates are presented below:

Scheme IX describes an illustrative synthesis of W-[Ring A]-[L¹]-[Ring B] in which Rings A and B are phenyl and L¹ is NHC(O).

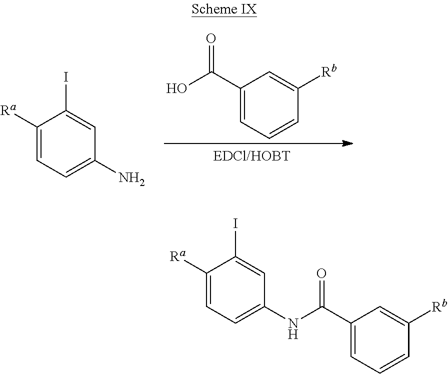

Scheme X depicts the synthesis of a variant of the foregoing in which Ring B is a 2-pyridine and L¹ is C(O)NH (i.e., in the other orientation).

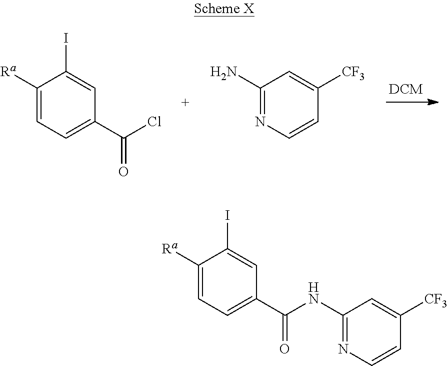

Schemes XI and XII, below, illustrate the synthesis of W-[Ring A]-[L¹]-[Ring B] in which Rings A and B are phenyl and Ring C is a heteroaryl ring. These intermediates are useful for making compounds of Formula II.

More specifically, Scheme XI describes the preparation of intermediates in which Ring C is an imidazole ring.

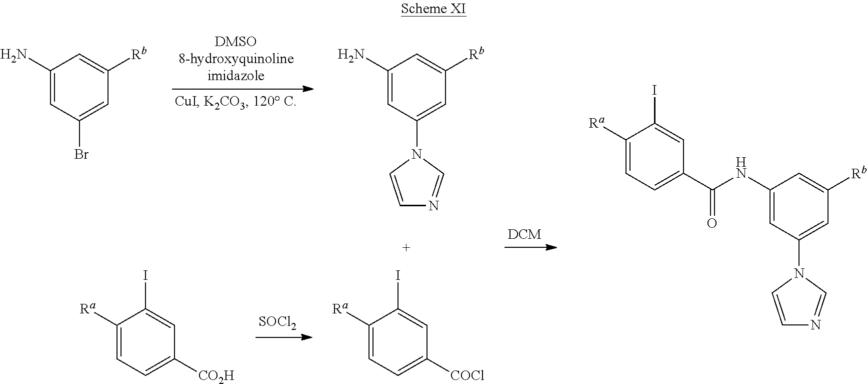

Scheme XII describes the preparation of intermediates in which Ring C is a pyrrole or an oxazole ring.

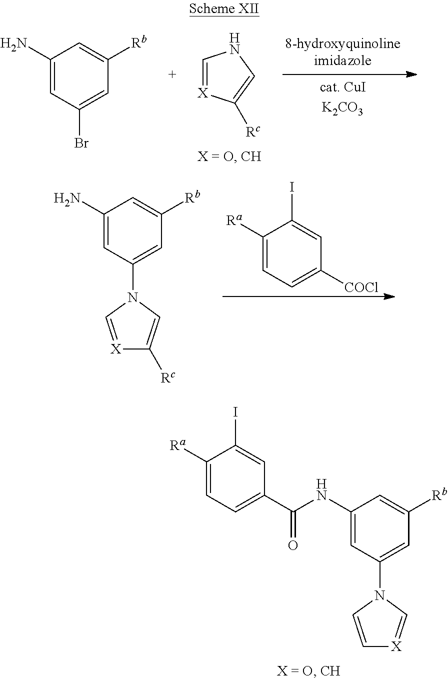

Scheme XIII illustrates the synthesis of W-[Ring A]-[L¹]-[Ring B] in which Rings A and B are phenyl and an R^{b} substituent is -L²-[Ring D]. These intermediates are useful for making compounds of Formula III in which Ring D is a 5 or 6-membered heterocycle, containing one or two heteroatoms.

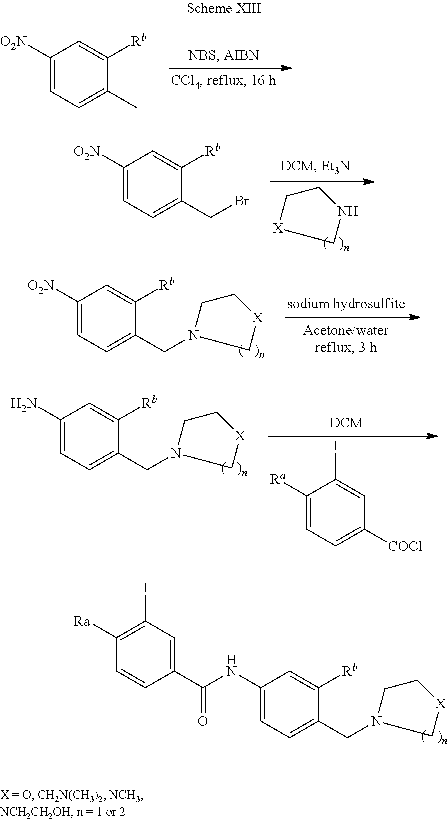

In this scheme, non-limiting examples of substituents R^{b} on Ring B are halo, e.g., Cl; lower alkyl groups, e.g., isopropyl; and substituted lower alkyl groups, e.g. —CF3; and non-limiting examples of Ring D are N,N-dimethylpyrrolidine, N-(2-hydroxyethyl)piperazine, and N-methylpiperazine.

Intermediates W-[Ring A]-[L¹]-[Ring B], such as those presented in the various synthetic schemes above, can be reacted with an acetylenic Ring T using the Sonogashira coupling conditions described in the general Scheme I.

An example is depicted below in Scheme XIV, in which Ring T moiety can be further derivatized after the Sonogashira coupling step, to generate various interesting substituted analogs of this invention.

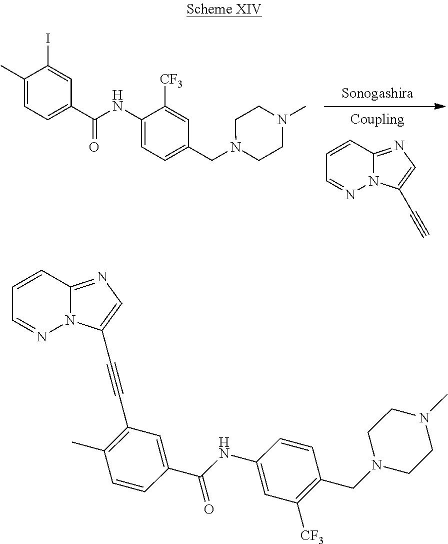

Alternatively, the W-[Ring A]-[L¹]-[Ring B] can be reacted under Sonogashira conditions with trimethylsilylacetylene, prior to the coupling with an iodo- or a bromo-activated Ring T as otherwise described in the general Scheme II.

An example is depicted in Scheme XV:

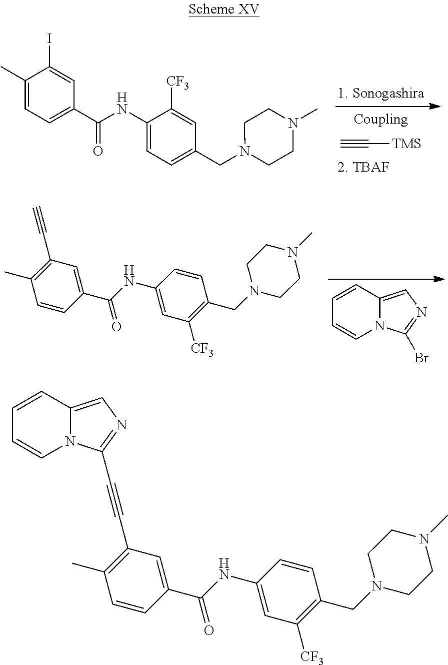

In other embodiments, the steps can be carried out in a different order. For example, the Sonogashira Coupling reaction can be used to Ring T to Ring A prior to linking that portion to Ring B and/or [Ring B]-[L²]-[Ring D] and/or [Ring B]-[Ring C] as shown in Scheme XVI.

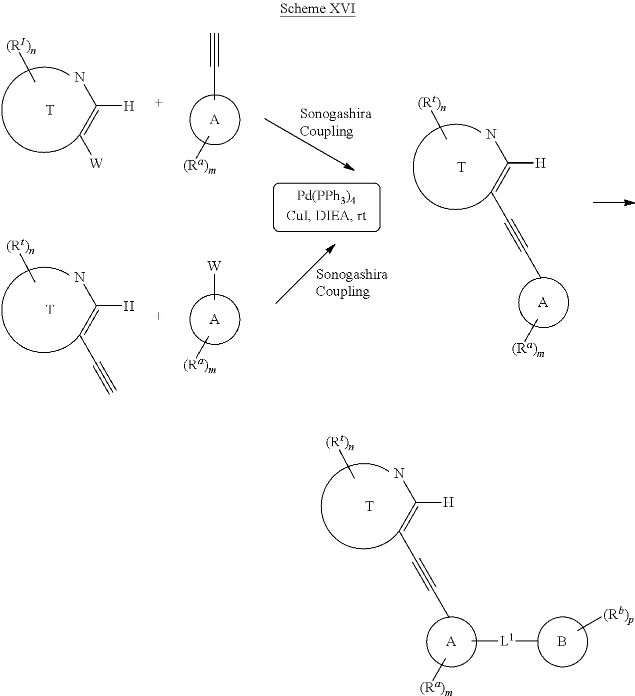

In a non-limiting example in which Ring A and Ring B are phenyl and L¹ is CONH, Scheme XVII describes Sonogashira Coupling of an acetylenic Ring T with 3-iodo-4-methylbenzoic acid (a Ring A moiety) to generate a [Ring T]-[Ring A] intermediate which then undergoes an amide coupling with an optionally substituted Ring B moiety:

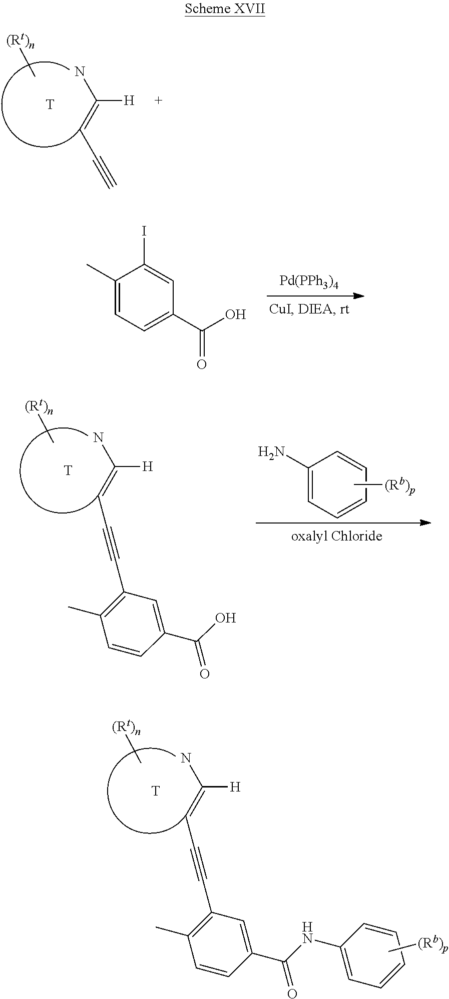

This approach is illustrated in Scheme XVIII which depicts the coupling of an acetylenic Ring T (i.e., 3-ethynylimidazo[1,2-b]pyridazine) with a substituted W-[Ring A] (i.e., 3-iodo-4-methylbenzoic acid), followed by an amide coupling of the resultant [Ring T]-[Ring A]-COOH intermediate with a H2N-[Ring B]-L2-[Ring C] moiety (i.e., 4-((4-methylpiperazin-1-yl)methyl)-3-(trifluoromethylaniline):

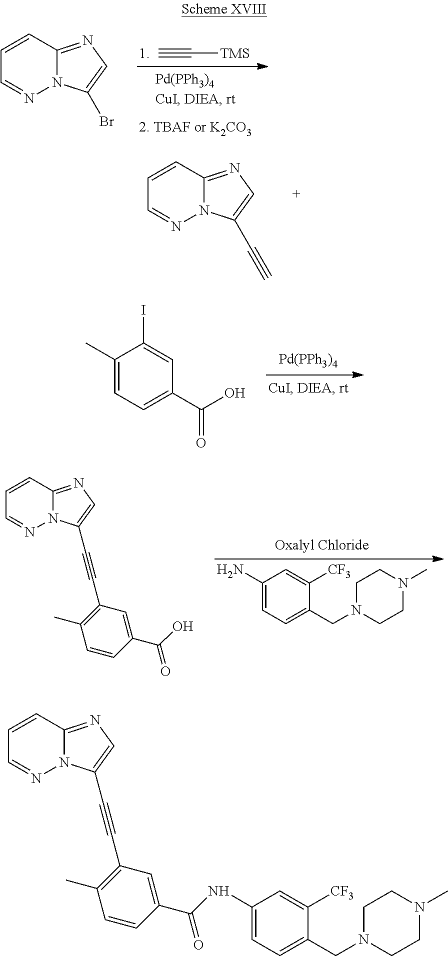

Alternatively, as another illustration of the practitioner's range of assembly options, the 3-iodo-4-methylbenzoic acid Ring A intermediate can be reacted in a Sonogashira reaction with trimethylsilylacetylene, which after silyl deprotection, can a second Sonogashira coupling reaction with an activated Ring T as illustrated in Scheme XIX.

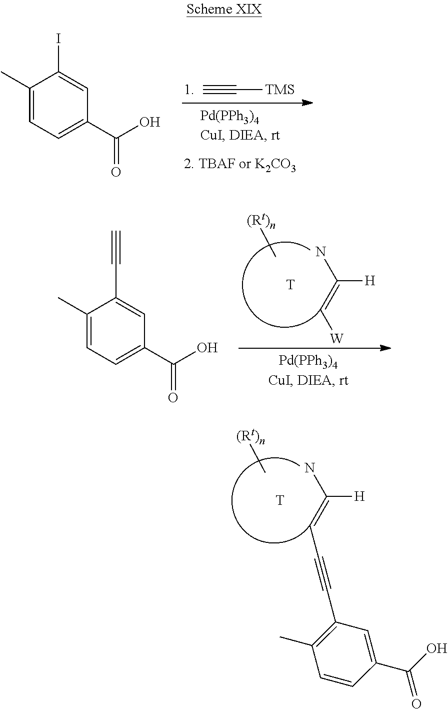

With synthetic approaches such as the foregoing, combined with the examples which follow, additional information provided herein and conventional methods and materials, the practitioner can prepare the full range of compounds disclosed herein.

In addition to the general synthetic approaches disclosed above, the synthesis of ponatinib free base and ponatinib hydrochloride have been specifically reported in Applicant's own WO 2011/053,938, which is incorporated here by reference. For the convenience of the reader, the synthetic scheme is reproduced immediately below.

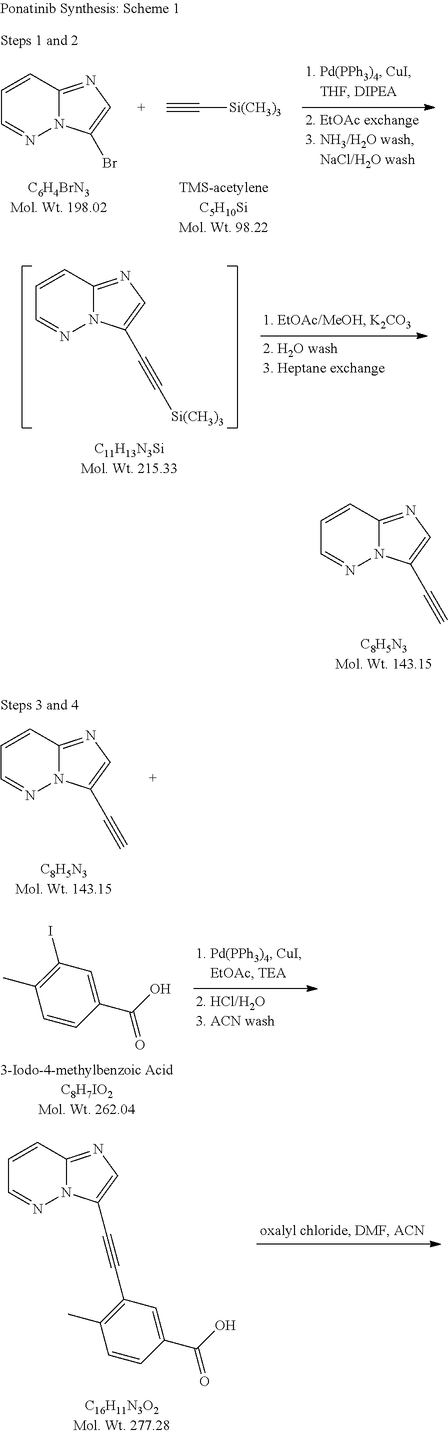

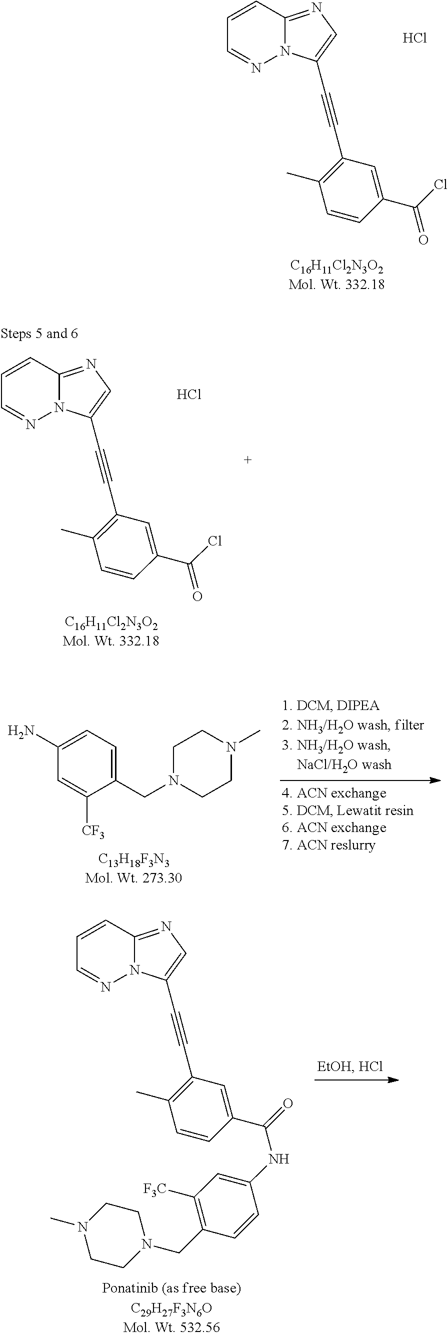 [SMILES: Cc1ccc(C(=O)Cl)cc1C#Cc1cnc2cccnn12 ; Cl ; Cc1ccc(C(=O)Cl)cc1C#Cc1cnc2cccnn12 ; Cl ; CN1CCN(Cc2ccc(N)cc2C(F)(F)F)CC1 ; Cc1ccc(C(=O)Nc2ccc(CN3CCN(C)CC3)c(C(F)(F)F)c2)cc1C#Cc1cnc2cccnn12]

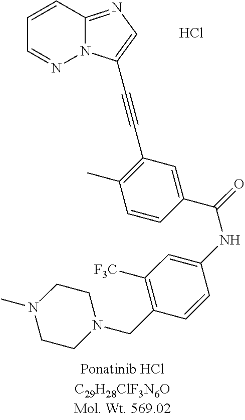 [SMILES: Cc1ccc(C(=O)Nc2ccc(CN3CCN(C)CC3)c(C(F)(F)F)c2)cc1C#Cc1cnc2cccnn12 ; Cl]

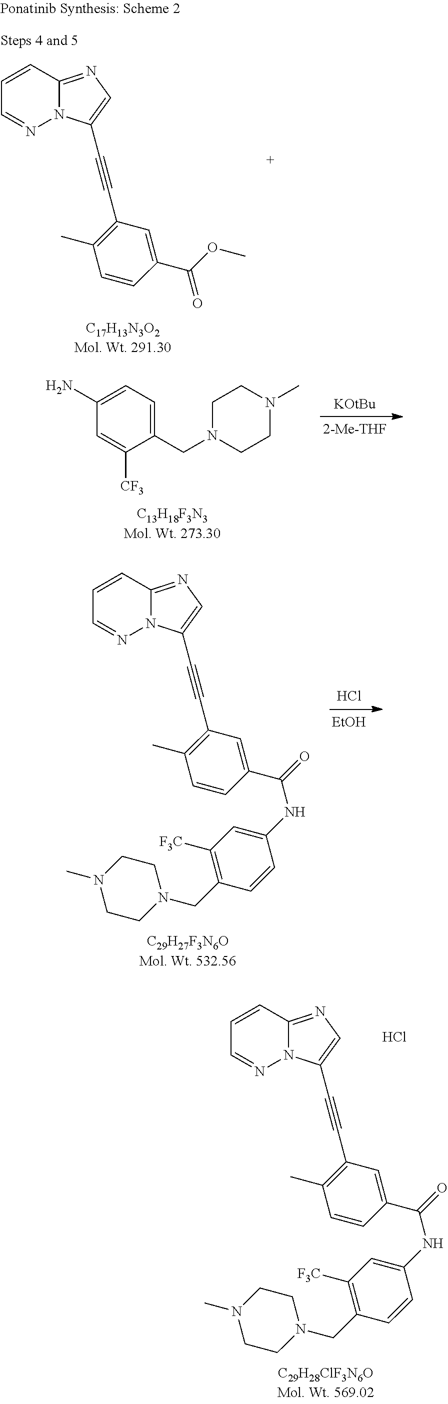

The mono-hydrochloride salt of ponatinib has been used for carrying out clinical trials. Further identifying information for ponatinib includes:

Chemical name: 3-(Imidazo[1,2-b]pyridazin-3-ylethynyl)-4-methyl-N-(4-((4-methylpiperazin-1-yl)methyl)-3-(trifluoromethyl)phenyl)benzamide, hydrochloride salt;

USAN: ponatinib;

USANM: ponatinib hydrochloride;

CAS Registry No.: 1114544-31-8 (HCl Salt) and 943319-70-8 (free base);

CAS Index name: Benzamide,3-(2-imidazo[1,2-b]pyridazin-3-ylethnyl)-4-methyl-N-[4-[(4-methyl-1-piperazinyl)methyl]-3-(trifluoromethyl)phenyl]-hydrochloride (1:1);

Molecular Formula: C₂₉H₂₈ClF₃N₆O(HCl salt) and C₂₉H₂₇F₃N₆O (free base) (no chiral centers); and

Molecular Weight: 569.02 g/mol (HCl salt) and 532.56 g/mol (free base).

### Exemplary Compounds of Formula I

Some of the compounds described in the following examples have been converted into an HCl salt. The general procedure for generating HCl salts is described below:

To the final product was added just enough MeOH saturated with HCl (g) to dissolve, cooled to 0° C. for 0.5-1 h, filtered, washed solid with ice cold MeOH then Et₂O, and the resulting solid dried in a vacuum desiccator to provide in most cases the tris HCl salt.

### Example 1

### N-(3-(1H-imidazol-1-yl)-5-(trifluoromethyl)phenyl)-3-(imidazo[1,2-a]pyrazin-3-ylethynyl)-4-methylbenzamide

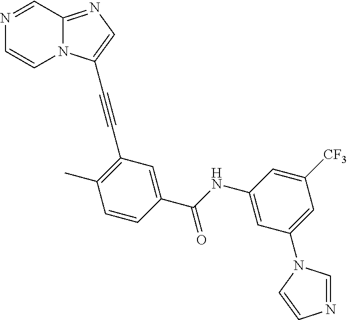 [SMILES: Cc1ccc(C(=O)Nc2cc(-n3ccnc3)cc(C(F)(F)F)c2)cc1C#Cc1cnc2cnccn12]

Imidazol[1,2-a]pyrazine: A solution of aminopyrazine (1 g, 10.5 mmol) and chloroacetaldehyde (50% wt in H₂O; 1.98 g, 12.6 mmol) in 1.6 mL of EtOH was heated at 90° C. in a sealed tube for 5 h. Upon cooling to ambient temperature, the reaction mixture was concentrated and diluted with dichloromethane (DCM). The organic layer washed with saturated aqueous NaHCO₃ then dried over MgSO₄ and concentrated. The crude product was purified by silica gel flash chromatography (eluted with 10% MeOH/DCM) to provide 0.8 g of product.

### 3-((Trimethylsilyl)ethynyl)imidazo[1,2-a]pyrazine

A mixture of 3-bromoimidazo[1,2-a]pyrazine (0.15 g, 0.76 mmol; prepared according to J. Bradac, et al. J. Org. Chem. (1977), 42, 4197-4201), 0.09 g (0.91 mmol) of ethynyltrimethylsilane, 0.044 g (0.038 mmol) of Pd(PPh₃)₄, 0.014 g (0.076 mmol) of CuI, and 0.26 mL (1.52 mmol) of diisopropylethylamine in 3.8 mL of DMF was heated at 50° C. overnight under an atmosphere of N₂. Upon cooling to ambient temperature, the reaction mixture was concentrated and the crude product was purified by silica gel flash chromatography (eluted with 50% EtOAc/hexanes) to provide 0.15 g of product: 216 m/z (M+H).

### 3-Ethynylimidazo[1,2-a]pyrazine

To a solution of 3-((Trimethylsilyl)ethynyl)imidazo[1,2-a]pyrazine (0.15 g, 0.7 mmol) in 3.5 mL of THF was added 1.05 mL (1.05 mmol) of tetrabutylammonium fluoride (1.0M in THF) at ambient temperature. The solution was stirred for 15 min, concentrated, and the crude product purified by silica gel flash chromatography (eluted with 50% EtOAc/hexanes) to provide 0.078 g of product.

### 3-(1H-imidazol-1-yl)-5-(trifluoromethyl)aniline

A mixture of 3-Amino-5-bromobenzotrifluoride (4.0 g, 0.0167 mol), 8-hydroxy quinoline (0.362 g, 0.0025 mol), CuI (0.476 g, 0.025 mol), imidazole (1.36 g, 0.0199 mol), and potassium carbonate (2.52 g, 0.0183 mol) in 17 mL of DMSO (degassed with argon for ˜10 min) was heated at 120° C. under an atmosphere of argon for 15 h; the HPLC indicated no starting material. A 14% aqueous solution of ammonium hydroxide was added to the cooled mixture and this was stirred for 1 h at ambient temperature. Water (50 mL) and EtOAc (200 mL) were added and the aqueous layer was extracted with EtOAc (3×30 mL). The combined organic layers were dried over Na₂SO₄ and concentrated. The crude product was purified by silica gel flash chromatography (eluted with EtOAc/hexanes) to provide 2.51 g of product.

### N-(3-(1H-imidazol-1-yl)-5-(trifluoromethyl)phenyl)-3-iodo-4-methylbenzamide

To 3-Iodo-4-methylbenzoic acid (3.07 g, 0.0117 mol) was added thionyl chloride (10 mL) and refluxed for 2 h. The excess thionyl chloride was carefully removed and the resulting acid chloride was dried in vacuo for 2 h. The residue was then dissolved in DCM (anhydrous, 25 mL) and cooled on ice. To the cooled solution was added 3-(1H-imidazol-1-yl)-5-(trifluoromethyl)aniline 5 (3.46 g, 0.0152 mol) in DCM followed by the dropwise addition of diisopropylethylamine (8.2 mL, 0.047 mol). This was stirred at ambient temperature for 21 h. The white solid that separated was filtered and washed with water and dried to provide 4.65 g of product. Additional product could be obtained from the filtrate following concentration and purification by silica gel flash chromatography in EtOAc/hexanes.

### N-(3-(1H-imidazol-1-yl)-5-(trifluoromethyl)phenyl)-3-(imidazo[1,2-a]pyrazin-3-ylethynyl)-4-methylbenzamide

A mixture of 3-Ethynylimidazo[1,2-a]pyrazine (0.075 g, 0.52 mmol), 0.245 g (0.52 mmol) of N-(3-(1H-imidazol-1-yl)-5-(trifluoromethyl)phenyl)-3-iodo-4-methylbenzamide, 0.030 g (0.026 mmol) of Pd(PPh₃)₄, 0.007 g (0.039 mmol) of CuI, and 0.14 mL (0.78 mmol) of diisopropylethylamine in 3.0 mL of DMF was stirred at ambient temperature overnight under an atmosphere of N₂. The reaction mixture was concentrated and the crude product was purified by silica gel flash chromatography (eluted with 10% EtOAc/hexanes, then 100% EtOAc, then 10% MeOH/EtOAc) to provide 0.090 g of product as a solid: 487 m/z (M+H).

### Alternative Synthesis of N-(3-(1H-imidazol-1-yl)-5-(trifluoromethyl)phenyl)-3-(imidazo[1,2-a]pyrazin-3-ylethynyl)-4-methylbenzamide

3-((Trimethylsilyl)ethynyl)imidazo[1,2-a]pyrazine can be prepared as described previously. In one variation, the reaction can also be carried out in THF instead of DMF. The crude product can also be purified by silica gel pad chromatography (eluted with ethyl acetate/hexane) and a brief treatment with activated charcoal (Darco) can be carried out to help further reduce contamination with the homo coupling product.

### 3-Ethynylimidazo[1,2-a]pyrazine

To a solution of 3-((trimethylsilyl)ethynyl) imidazo[1,2-a]pyrazine (1.39 mol) in 10× volume of Ethyl acetate and 1.5× volume of Methanol is added two and a half equivalents of potassium carbonate at ambient temperature and the solution stirred for 1 hour. Potassium carbonate is filtered off and the organic stream is washed with water and with saturated sodium chloride solution (two or more times). Aqueous phases can be combined and re-extracted with ethyl acetate. Organic streams can then be combined and concentrated under vacuum to about 0.5 L. Solids can be allowed to precipitate out upon concentration. Slurry is cooled, e.g. to about −5° C., stored overnight, filtered, and washed with about 0.3 L of cold ethyl acetate. The solids can then be dried under vacuum.

3-(imidazo[1,2-a]pyrazin-3-ylethynyl)-4-methylbenzoic acid can be prepared in a manner similar to that described above for the Sonogashira reaction. 3-Ethynylimidazo[1,2-a]pyrazine and 3-iodo-4-methylbenzoic acid are used as coupling partners. Alternatively, the solvent (DMF) can be replaced by ethyl acetate and the base (Hunig base) can be replaced by triethylamine. The product can be isolated by filtration of the crude reaction mixture. The filter cake is washed sequentially with a solvent such as ethyl acetate and then water, then dried in a vacuum oven. Further purification can be achieved by slurrying the solids in water adjusted to pH 3 with the addition of concentrated HCl. After filtration and water wash, the product can be dried in a vacuum oven.

### N-(3-(1H-imidazol-1-yl)-5-(trifluoromethyl)phenyl)-3-(imidazo[1,2-a]pyrazin-3-ylethynyl)-4-methylbenzamide

3-(imidazo[1,2-a]pyrazin-3-ylethynyl)-4-methylbenzoic acid (18 mmol) is dissolved in methylene chloride (100 mL). To this solution is added 3 equivalents of 4-methylmorpholine (NMM) followed by 1.05 equivalents of oxalyl chloride. After stirring at ambient temperature for 30 minutes, 0.8 equivalents of 3-(1H-imidazol-1-yl)-5-(trifluoromethyl)aniline (prepared as above) is added along with 5 mole % of DMAP. After initially stirring at ambient temperature, the mixture is brought to reflux and stirred overnight. After 16 h an additional 0.2 equivalents of the aniline is added, bringing the total charge to 1 equivalent. The mixture can then be stirred for an additional 2 h, quenched with water, and the layers separated. The aqueous layer can be extracted with methylene chloride (2×50 mL) and the combined extracts can be washed with water. The combined methylene chloride layers can then be evaporated and the residue dissolved in 100 mL of ethyl acetate (20 mL). After standing for 1 h, the product is allowed to crystallize. The mixture is cooled, e.g. to 0° C., filtered, and the solid product is washed with cold ethyl acetate.

### N-(3-(1H-imidazol-1-yl)-5-(trifluoromethyl)phenyl)-3-(imidazo[1,2-a]pyrazin-3-ylethynyl)-4-methylbenzamide mono hydrochloride salt

N-(3-(1H-imidazol-1-yl)-5-(trifluoromethyl)phenyl)-3-(imidazo[1,2-a]pyrazin-3-ylethynyl)-4-methylbenzamide (0.94 mmol) can be suspended in MeCN (10 ml) and heated with stirring to a temperature of 45 to 55° C. (hot plate temperature). Hydrochloric acid (1.1 eq 1M solution in EtOH) is added to obtain dissolution. Within a few minutes, a precipitate is allowed to form. The suspension can be cooled to ambient temperature and then filtered and washed with MeCN (1×1.5 ml liquors+1×1.5 ml fresh). The solid can be dried at 50° C. under vacuum to constant weight.

### Example 2

### 3-(Imidazo[1,2-a]pyrazin-3-ylethynyl)-4-methyl-N-(4-((4-methylpiperazin-1-yl)methyl)-3-(trifluoromethyl)phenyl)benzamide

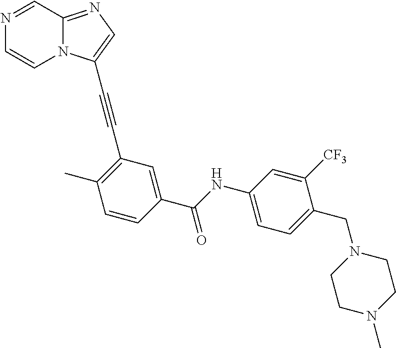 [SMILES: Cc1ccc(C(=O)Nc2ccc(CN3CCN(C)CC3)c(C(F)(F)F)c2)cc1C#Cc1cnc2cnccn12]

The title compound was synthesized from 3-ethynylimidazo[1,2-a]pyrazine and 3-iodo-4-methyl-N-(4-((4-methylpiperazin-1-yl)methyl)-3-(trifluoromethyl)phenyl)benzamide in a manner similar to that described for Example 1. The product was obtained as a solid: 533 m/z (M+H).

### 1-(Bromomethyl)-4-nitro-2-(trifluoromethyl)benzene

A suspension of 2-methyl-5-nitrobenzotrifluoride (3.90 g, 19 mmol), N-bromosuccinimide (NBS, 3.56 g, 20 mmol), 2,2′-azobis(2-methylpropionitrile) (AIBN, 94 mg, 0.6 mmol) in CCl₄ (40 mL) was refluxed under N₂ for 16 h. HPLC indicated ca. 50% conversion. More NBS (10 mmol) and AIBN (0.6 mmol) was added, and the mixture was refluxed for another 14 h. HPLC indicated ca. 80% conversion. The reaction mixture was cooled down, and the solid was filtered off and washed with EtOAc. The combined filtrate was washed with aq. NaHCO₃, dried over Na₂SO₄, filtered, concentrated on rotovap and further dried under vacuum. ¹H NMR shows the ratio of desired product to unreacted 2-methyl-5-nitrobenzotrifluoride is 75:25. This material was not purified but used directly in the next step.

### 1-Methyl-4-(4-nitro-2-(trifluoromethyl)benzyl)piperazine

To a solution of crude 1-(bromomethyl)-4-nitro-2-(trifluoromethyl)benzene (13.33 mmol, 75% pure) in DCM (10 mL) was added Et₃N (1.4 mL, 10 mmol) and 1-methylpiperazine (1.1 mL, 10 mmol). After stirring for 3 h at rt, aq. NaHCO₃ was added, and the mixture was extracted with DCM. The combined organic layer was dried over Na₂SO₄, filtered, concentrated, and the resulting residue was purified by silica gel chromatography (eluted with 10% MeOH/DCM) to provide 2.21 g of product as a pale yellow oil.

### 4-((4-Methylpiperazin-1-yl)methyl)-3-(trifluoromethyl)aniline

A suspension of 1-methyl-4-(4-nitro-2-(trifluoromethyl)benzyl)piperazine (1.23 g, 4 mmol) and sodium hydrosulfite (7.0 g, 85% pure from Aldrich, 40 mmol) in acetone and water (1:1, 20 mL) was refluxed for 3 h. Upon cooling, the volatile components (mainly acetone) were removed on rotavap, and the resulting mixture was subjected to filtration. The solid was thoroughly washed with EtOAc. The combined filtrate was extracted with n-BuOH (4×), and the combined organic layer was washed with saturated aq. NaHCO₃, dried (Na₂SO₄), filtered, concentrated, and the resulting residue was purified by silica gel chromatography (eluted with 5% MeOH/DCM, MeOH was pre-saturated with ammonia gas) to provide 0.71 g of product as a pale yellow solid.

### 3-Iodo-4-methyl-N-(4-((4-methylpiperazin-1-yl)methyl)-3-(trifluoromethyl)phenyl)Benzamide

3-Iodo-4-methylbenzoyl chloride (0.48 g, 1.7 mmol), prepared from the reaction of 3-iodo-4-methylbenzoic acid and SOCl₂ (as previously described), was added to a solution of 4-((4-methylpiperazin-1-yl)methyl)-3-(trifluoromethyl)aniline (0.47 g, 1.7 mmol), N,N-diisopropylethylamine (0.26 g, 2.0 mmol), and a catalytic amount of DMAP in THF (10 mL). After stirring at rt for 2 h, the reaction was quenched with water. EtOAc was added and the layers separated. The combined organic layers were concentrated to dryness and purified by silica gel chromatography (eluted with 5% MeOH/DCM, MeOH was pre-saturated with ammonia gas), to provide 0.51 g of product as an off-white solid.

### Alternative synthesis of 3-(Imidazo[1,2-a]pyrazin-3-ylethynyl)-4-methyl-N-(4-((4-methylpiperazin-1-yl)methyl)-3-(trifluoromethyl)phenyl)benzamide

3-(Imidazo[1,2-a]pyrazin-3-ylethynyl)-4-methyl-N-(4-((4-methylpiperazin-1-yl)methyl)-3-(trifluoromethyl)phenyl)benzamide and its mono hydrochloride salt can be prepared in an alternative synthesis similar to that described in Example 1 from 3-(imidazo[1,2-a]pyrazin-3-ylethynyl)-4-methylbenzoic acid and 4-((4-methylpiperazin-1-yl)methyl)-3-(trifluoromethyl)aniline (as prepared above).

### Example 3

### N-(3-(2-((dimethylamino)methyl)-1H-imidazol-1-yl)-5-(trifluoromethyl)phenyl)-3-(imidazo[1,2-a]pyrazin-3-ylethynyl)-4-methylbenzamide

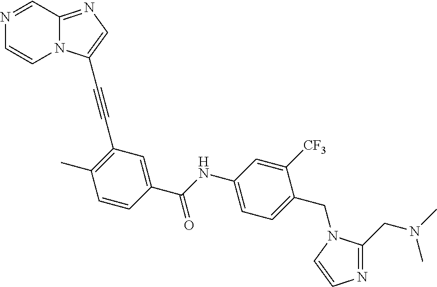 [SMILES: Cc1ccc(C(=O)Nc2ccc(Cn3ccnc3CN(C)C)c(C(F)(F)F)c2)cc1C#Cc1cnc2cnccn12]

The title compound was synthesized from 3-ethynylimidazo[1,2-a]pyrazine and N-(3-(2-((dimethylamino)methyl)-1H-imidazol-1-yl)-5-(trifluoromethyl)phenyl)-3-iodo-4-methylbenzamide in a manner similar to that described for Example 1. The product was obtained as a solid: 544 m/z (M+H).

### 1-(1H-imidazol-2-yl)-N,N-dimethylmethanamine

To a two-necked round-bottomed flask equipped with a reflux condenser and a pressure-equalizing addition funnel, was added 2-imidazolecarboxaldehyde (6 g, 62.5 mmol) in MeOH (60 mL). To this suspension (ambient temperature) was added a solution of dimethylamine (40% aqueous, 60 mL) at a fast dropping rate (20 min). After the addition was complete, solid sodium borohydride (7 g, 186.8 mmol), was CAUTIOUSLY added portionwise over 45 min. Foaming occurred after each portion, and the internal temperature was allowed to maintain ˜50° C. without external cooling. The reaction mixture was then heated to 65° C. for 3 h and allowed to cool to ambient temperature for overnight. The reaction contents were concentrated in vacuo and the resultant residue was taken up in EtOAc (2×30 mL) washed with brine and with CHCl₃ (4×100 mL). The EtOAc extract was discarded. The CHCl₃ extract was dried over (NaSO₄), filtered, and concentrated in vacuo to give 3.7 g of the desired product as a waxy solid.

### 3-(2-((Dimethylamino)methyl)-1H-imidazol-1-yl)-5-(trifluoromethyl)aniline

3-Amino-5-bromobenzotrifluoride (6 g, 25 mmol) and 1-(1H-imidazol-2-yl)-N,N-dimethylmethanamine (3.7 g, 29.6 mmol) were dissolved in anhydrous DMSO (25 mL). To this was added CuI (0.95 g, 7.5 mmol), 8-hydroxy quinoline (0.72 g, 7.5 mmol) and K₂CO₃ (6.9 g, 50 mmol). The mixture was stirred vigorously and degassed with N₂ for 15 minutes. The flask was then equipped with a condenser and heated at 120° C. for 18 h. The resultant heterogeneous mixture was cooled to rt, poured into 14% aq. NH₄OH (100 mL) and extracted with EtOAc (3×300 ml). The combined extracts were dried over NaSO₄ and concentrated in vacuo. The residue was chromatographed over silica gel eluting with MeOH/DCM (5:95) to furnish 3.5 g of the desired product as a tan colored material: 285 m/z (M+H).

### N-(3-(2-((dimethylamino)methyl)-1H-imidazol-1-yl)-5-(trifluoromethyl)phenyl)-3-iodo-4-methylbenzamide

3-Iodo-4-methylbenzoyl chloride (2.2 g, 7.88 mmol), dissolved in anhydrous THF (13 mL), was added dropwise to a solution of 3-(2-((dimethylamino)methyl)-1H-imidazol-1-yl)-5-(trifluoromethyl)aniline (1.5 g, 5.5 mmol), DIPEA (2.1 mL, 11.8 mmol) in THF (30 mL) at ˜5° C. The resultant solution was stirred at ambient temperature overnight. The solvent was removed in vacuo and the crude residue was redissolved in CH₂Cl₂ and washed with 1N NaOH. The organic layer was then washed with water, and brine then dried over NaSO₄ before being concentrated in vacuo. The brown colored residue was then triturated in a mixture of hexanes/DCM to precipitate 1.4 g of the desired product as an off-white powder: 529 m/z (M+H).

### Alternative Synthesis of N-(3-(2-((dimethylamino)methyl)-1H-imidazol-1-yl)-5-(trifluoromethyl)phenyl)-3-(imidazo[1,2-a]pyrazin-3-ylethynyl)-4-methylbenzamide

N-(3-(2-((dimethylamino)methyl)-1H-imidazol-1-yl)-5-(trifluoromethyl)phenyl)-3-(imidazo[1,2-a]pyrazin-3-ylethynyl)-4-methylbenzamide and its mono hydrochloride salt can be prepared in an alternative synthesis similar to that described in Example 1 from 3-(imidazo[1,2-a]pyrazin-3-ylethynyl)-4-methylbenzoic acid and 3-(2-((Dimethylamino)methyl)-1H-imidazol-1-yl)-5-(trifluoromethyl)aniline (as prepared above).

### Example 4

### 3-(Imidazo[1,2-a]pyridin-3-ylethynyl)-4-methyl-N-(3-(4-methyl-1H-imidazol-1-yl)-5-(trifluoromethyl)phenyl)benzamide

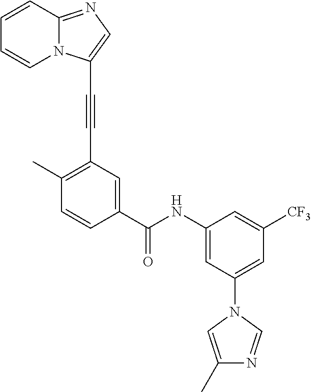 [SMILES: Cc1cn(-c2cc(NC(=O)c3ccc(C)c(C#Cc4cnc5ccccn45)c3)cc(C(F)(F)F)c2)cn1]

### 3-Ethynylimidazo[1,2-a]pyridine

To 3-bromoimidazo[1,2-a]pyridine (5 g, 0.0254 mol) in acetonitrile (50 mL) in a sealed tube was added bis(triphenylphosphine) palladium(II) dichloride (0.445 g, 0.634 mmol), CuI (0.17 g, 0.89 mmol), dicyclohexylamine (5.6 mL, 0.028 mol) and ethynyltrimethylsilane (7.2 mL, 0.051 mol). The solution was purged with argon for 15 minutes, sealed and heated at 80° C. for 3 h. At this point the HPLC did not show any starting bromide. The solvents were concentrated and to the residue was added water and dichloromethane (25 mL each). The organic layer was separated and the aqueous layer was repeatedly extracted with dichloromethane (3×20 mL). The combined extracts were dried (Na₂SO₄), and concentrated (Rf, 0.47 in 1/1 hexanes/ethyl acetate). The resulting residue was dissolved in THF (100 mL) and treated with tetrabutyl ammonium fluoride monohydrate (8.3 g, 0.032 mol) in water (5 mL) and the mixture was stirred at rt for 2 h. The solvents were concentrated and the resulting residue was partitioned between water (25 mL) and dichloromethane (150 mL). The aqueous layer was extracted with dichloromethane (2×30 mL). The combined extracts were dried (Na₂SO₄), and concentrated. The resulting residue was purified by combiflash on silica gel using hexanes/ethyl acetate. The desired product was eluted with 50/50 hexane/ethyl acetate and isolated as an off-white solid: MS (M+H)⁺200.

### 3-(4-Methyl-1H-imidazol-1-yl)-5-(trifluoromethyl)aniline

A suspension of 3-bromo-5-(trifluoromethyl)aniline (4.8 g, 20 mmol), 4-methylimidazole (1.97 g, 24 mmol), potassium carbonate (3.04 g, 22 mmol), CuI (0.57 g, 3 mmol), and 8-hydroxyquinoline (0.44 g, 3 mmol), in dry DMSO (20 mL) in a pressure tube was degassed by bubbling N₂ into the suspension for 10 minutes while stirring. The tube was sealed tightly. The mixture was heated at 120° C. (oil bath temperature) for 15 h. The mixture was cooled down to 45-50° C. and 14% aq. NH₄OH (20 mL) was added. The mixture was maintained at this temperature for 1 h. After cooling to rt, water and ethyl acetate were added. The aqueous layer was extracted with ethyl acetate and the combined organic layers were passed through a short silica gel column to remove most of green/blue Cu salts. The filtrate was dried over sodium sulfate and concentrated on a rotavap. The crude product was recrystallized from EtOAc/hexanes, giving pure pale yellow needles. The mother liquor was concentrated and the residue was purified on silica gel column (5% methanol/methylene chloride), yielding a second crop as pale yellow needles.

### 3-Indo-4-methyl-N-(3-(4-methyl-1H-imidazol-1-yl)-5-(trifluoromethyl)phenyl)Benzamide

3-Iodo-4-methylbenzoic acid (2.62 g, 10 mmol) was refluxed in SOCl₂ (10 mL) for 1 h. The volatile components were removed on a rotavap and the residue was dissolved in benzene (10 mL), concentrated to dryness on a rotavap and further dried under vacuum. The resulting acyl chloride was added to a solution 3-(4-methyl-1H-imidazol-1-yl)-5-(trifluoromethyl)benzeneamine (2.46 g, 10.2 mmol), N,N-diisopropylethylamine (1.56 g, 12 mmol), and a catalytic amount of DMAP in THF (20 mL). After stirring at rt for 2 h, the reaction was quenched with water. EtOAc was added and the layers separated. The combined organic layers were concentrated to dryness and used without purification in next step.

### 3-(Imidazo[1,2-a]pyridin-3-ylethynyl)-4-methyl-N-(3-(4-methyl-1H-imidazol-1-yl)-5-(trifluoromethyl)phenyl)benzamide

To a solution of 3-iodo-4-methyl-N-(3-(4-methyl-1H-imidazol-1-yl)-5-(trifluoromethyl)phenyl)benzamide (0.11 g, 0.22 mmol.) in DMF (1 mL) in a sealed tube was added Pd[(PPh₃)₄] (0.013 g, 0.011 mmol), CuI (3 mg, 0.016 mmol), diethylisopropylamine (0.057 mL, 0.33 mmol.), followed by 3-ethynylimidazo[1,2-a]pyridine (0.040 g, 0.28 mmol.). The mixture was purged with argon for 15 minutes, sealed and stirred at rt for 28 h. The solvent was concentrated and the residue was taken up in methylene chloride (50 mL). The organic layer was washed with water, dried (Na₂SO₄) and evaporated to leave a brown residue which was purified by combiflash (hexane/ethyl acetate/methanol) to yield the desired material: MS (M+H)⁺500.

### Alternative Synthesis of 3-(Imidazo[1,2-a]pyridin-3-ylethynyl)-4-methyl-N-(3-(4-methyl-1H-imidazol-1-yl)-5-(trifluoromethyl)phenyl)benzamide

3-(Imidazo[1,2-a]pyridin-3-ylethynyl)-4-methyl-N-(3-(4-methyl-1H-imidazol-1-yl)-5-(trifluoromethyl)phenyl)benzamide and its mono hydrochloride salt can be prepared in an alternative synthesis similar to that described in Example 1 from 3-(imidazo[1,2-a]pyridin-3-ylethynyl)-4-methylbenzoic acid and 3-(4-Methyl-1H-imidazol-1-yl)-5-(trifluoromethyl)aniline (as prepared above). The 3-(imidazo[1,2-a]pyridin-3-ylethynyl)-4-methylbenzoic acid is prepared in a manner similar to that described in Example 1 using 3-Ethynylimidazo[1,2-a]pyridine and 3-iodo-4-methylbenzoic acid as Sonogashira coupling partners.

### Example 5

### N-(3-(1H-imidazol-1-yl)-5-(trifluoromethyl)phenyl)-3-(imidazo[1,2-a]pyridin-3-ylethynyl)-4-methylbenzamide

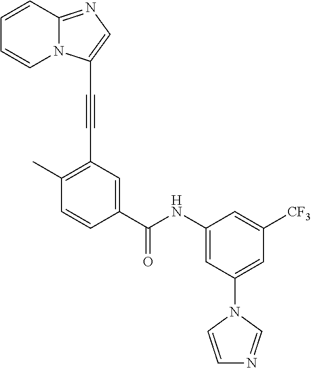 [SMILES: Cc1ccc(C(=O)Nc2cc(-n3ccnc3)cc(C(F)(F)F)c2)cc1C#Cc1cnc2ccccn12]

The titled compound was made as for example 1 using N-(3-(1H-imidazol-1-yl)-5-(trifluoromethyl)phenyl)-3-iodo-4-methylbenzamide and 3-ethynylimidazo[1,2-a]pyridine: MS (M+H)⁺486. The titled compound can also be prepared according to the alternative synthesis described in example 1 from 3-(imidazo[1,2-a]pyridin-3-ylethynyl)-4-methylbenzoic acid and 3-(1H-imidazol-1-yl)-5-(trifluoromethyl)aniline (as prepared in Example 1). The 3-(imidazo[1,2-a]pyridin-3-ylethynyl)-4-methylbenzoic acid is prepared in a manner similar to that described in Example 1 using 3-Ethynylimidazo[1,2-a]pyridine and 3-iodo-4-methylbenzoic acid as Sonogashira coupling partners.

### Example 6

### 3-(Imidazo[1,2-a]pyridin-3-ylethynyl)-4-methyl-N-(4-(trifluoromethyl)pyridin-2-yl)benzamide

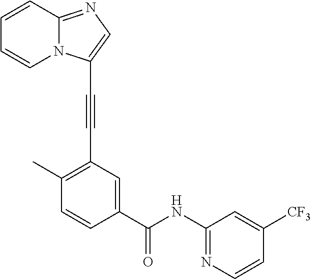 [SMILES: Cc1ccc(C(=O)Nc2cc(C(F)(F)F)ccn2)cc1C#Cc1cnc2ccccn12]

The titled compound was made as for example 1 using 3-iodo-4-methyl-N-(4-(trifluoromethyl)pyridin-2-yl)benzamide and 3-ethynylimidazo[1,2-a]pyridine: MS (M+H)⁺42.139.

### Example 7

### N-(5-tert-butylisoxazol-3-yl)-3-(imidazo[1,2-a]pyridin-3-ylethynyl)-4-methylbenzamide

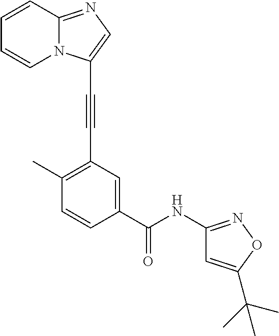 [SMILES: Cc1ccc(C(=O)Nc2cc(C(C)(C)C)on2)cc1C#Cc1cnc2ccccn12]

The titled compound was made as for example 1 using N-(5-tert-butylisoxazol-3-yl)-3-iodo-4-methylbenzamide and 3-ethynylimidazo[1,2-a]pyridine: MS (M+H)⁺399.

### Example 8

### 3-(Imidazo[1,2-a]pyridin-3-ylethynyl)-4-methyl-N-(4-((4-methylpiperazin-yl)methyl)-3-(trifluoromethyl)phenyl)benzamide

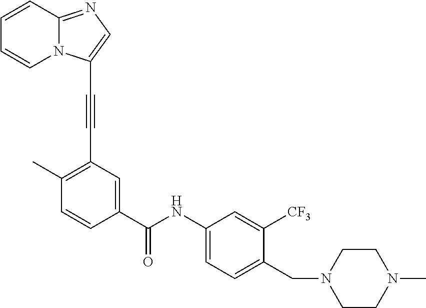 [SMILES: Cc1ccc(C(=O)Nc2ccc(CN3CCN(C)CC3)c(C(F)(F)F)c2)cc1C#Cc1cnc2ccccn12]

3-Ethynylimidazo[1,2-a]pyridine (37 mg, 0.26 mmol), 3-iodo-4-methyl-N-(4-((4-methylpiperazin-1-yl)methyl)-3-(trifluoromethyl)phenyl)benzamide (103.4 mg, 0.2 mmol), (prepared as in Example 2), Pd[(PPh₃)₄] (11.6 mg, 5 mol %), and CuI (2.9 mg, 7.5 mmol %) was placed in a vial with rubber septum. The mixture underwent 3 cycles of vacuum/filling with N₂, and DMF (1.5 ml) and N,N-diisopropylethylamine (53 mL, 0.3 mmol) was added. The mixture was stirred at rt for 16 h, and the reaction was quenched with H₂O. EtOAc and more water were added for extraction. The combined organic layer was dried (Na₂SO₄), filtered, concentrated, and the resulting residue was purified by silica gel chromatography (eluent: 5% MeOH in methylene chloride, MeOH was pre-saturated with ammonia gas), giving the titled compound as an off-white solid (53%, 56 mg): MS (M+H)⁺532.

### Alternative Synthesis of 3-(Imidazo[1,2-a]pyridin-3-ylethynyl)-4-methyl-N-(4-((4-methylpiperazin-1-yl)methyl)-3-(trifluoromethyl)phenyl)benzamide

3-(Imidazo[1,2-a]pyridin-3-ylethynyl)-4-methyl-N-(4-((4-methylpiperazin-1-yl)methyl)-3-(trifluoromethyl)phenyl)benzamide and its mono hydrochloride salt can be prepared in an alternative synthesis similar to that described in Example 1 from 3-(imidazo[1,2-a]pyridin-3-ylethynyl)-4-methylbenzoic acid and 4-((4-methylpiperazin-1-yl)methyl)-3-(trifluoromethyl)aniline (as prepared in example 2). The 3-(imidazo[1,2-a]pyridin-3-ylethynyl)-4-methylbenzoic acid is prepared in a manner similar to that described in Example 1 using 3-Ethynylimidazo[1,2-a]pyridine and 3-iodo-4-methylbenzoic acid as Sonogashira coupling partners.

### Example 9

### N-(3-(2-((dimethylamino)methyl)-1H-imidazol-1-yl)-5-(trifluoromethyl)phenyl)-3-(imidazo[1,2-a]pyridin-3-ylethynyl)-4-methylbenzamide

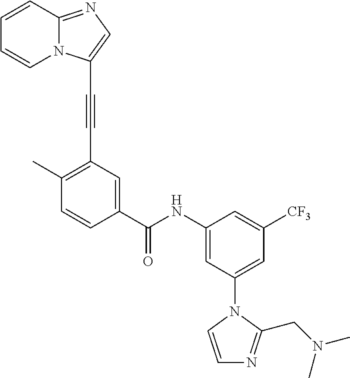 [SMILES: Cc1ccc(C(=O)Nc2cc(-n3ccnc3CN(C)C)cc(C(F)(F)F)c2)cc1C#Cc1cnc2ccccn12]

To 3-ethynylimidazo[1,2-a]pyridine (0.032 g, 0.22 mmol) in anhydrous DMF (1.26 mL) was added N-(3-(2-((dimethylamino)methyl)-1H-imidazol-1-yl)-5-(trifluoromethyl)phenyl)-3-iodo-4-methylbenzamide (prepared as in Example 3), Pd(PPh₃)₄ (0.013 g, 0.011 mmol), CuI (0.0032 mg, 0.0165 mmol) and DIPEA (0.064 mL, 0.44 mmol). The solution was degassed with argon for 15 minutes then stirred overnight at rt. The solvent was removed and the resultant residue was chromatographed over silica gel eluting initially with EtOAc and then with methanol/methylene chloride (5:95) to furnish the desired product: (0.07 g, 59%) MS (M+H)⁺542.

### Alternative Synthesis of N-(3-(2-((dimethylamino)methyl)-1H-imidazol-1-yl)-5-(trifluoromethyl)phenyl)-3-(imidazo[1,2-a]pyridin-3-ylethynyl)-4-methylbenzamide

N-(3-(2-((dimethylamino)methyl)-1H-imidazol-1-yl)-5-(trifluoromethyl)phenyl)-3-(imidazo[1,2-a]pyridin-3-ylethynyl)-4-methylbenzamide and its mono hydrochloride salt can be prepared in an alternative synthesis similar to that described in Example 1 from 3-(imidazo[1,2-a]pyridin-3-ylethynyl)-4-methylbenzoic acid and 3-(2-((Dimethylamino)methyl)-1H-imidazol-1-yl)-5-(trifluoromethyl)aniline (as prepared in Example 3). The 3-(imidazo[1,2-a]pyridin-3-ylethynyl)-4-methylbenzoic acid is prepared in a manner similar to that described in Example 1 using 3-Ethynylimidazo[1,2-a]pyridine and 3-iodo-4-methylbenzoic acid as Sonogashira coupling partners.

### Example 10

### 3-((8-Acetamidoimidazo[1,2-a]pyridin-3-yl)ethynyl)-4-methyl-N-(4-(trifluoromethyl)pyridin-2-yl)benzamide

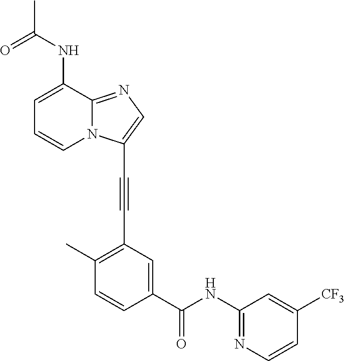 [SMILES: CC(=O)Nc1cccn2c(C#Cc3cc(C(=O)Nc4cc(C(F)(F)F)ccn4)ccc3C)cnc12]

### N-(3-Ethynylimidazo[1,2-a]pyridin-8-yl)acetamide

N-(3-Ethynylimidazo[1,2-a]pyridin-8-yl)acetamide was synthesized as for example IA from N-(3-bromoimidazo[1,2-a]pyridin-8-yl)acetamide (E. Smakula Hand and William W. Paudler, J. Org. Chem., 1978, 43, 2900-2906). The titled compound was isolated as an off-white solid, Rf, 0.6 (hexane/ethylacetate 50/50): MS (M+H)⁺200.

### 3-((8-Acetamidoimidazo[1,2-a]pyridin-3-yl)ethynyl)-4-methyl-N-(4-(trifluoromethyl)pyridin-2-yl)benzamide

The titled compound was made as for example 1 using 3-iodo-4-methyl-N-(4-(trifluoromethyl)pyridin-2-yl)benzamide and N-(3-ethynylimidazo[1,2-a]pyridin-8-yl)acetamide: MS (M+H)⁺478.4.

### Example 11

### N-(3-(1H-imidazol-1-yl)-5-(trifluoromethyl)phenyl)-3-((8-acetamidoimidazo[1,2-a]pyridin-3-yl)ethynyl)-4-methylbenzamide

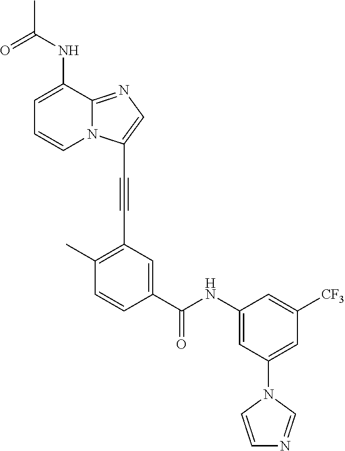 [SMILES: CC(=O)Nc1cccn2c(C#Cc3cc(C(=O)Nc4cc(-n5ccnc5)cc(C(F)(F)F)c4)ccc3C)cnc12]

The titled compound was made as for example 10 using N-(3-(1H-imidazol-1-yl)-5-(trifluoromethyl)phenyl)-3-iodo-4-methylbenzamide and N-(3-ethynylimidazo[1,2-a]pyridin-8-yl)acetamide: MS (M+H) 543.

### Example 12

### 4-Methyl-3-((8-(4-(methylsulfonyl)phenylamino)imidazo[1,2-a]pyridin-3-yl)ethynyl)-N-(4-(trifluoromethyl)pyridin-2-yl)benzamide

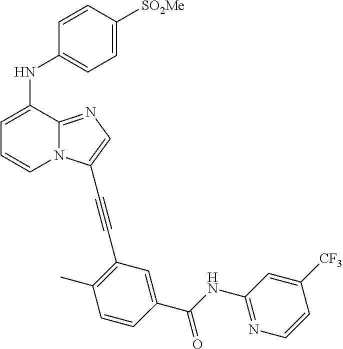 [SMILES: Cc1ccc(C(=O)Nc2cc(C(F)(F)F)ccn2)cc1C#Cc1cnc2c(Nc3ccc(S(C)(=O)=O)cc3)cccn12]

### 8-(Benzyloxy)-3-bromoimidazo[1,2-a]pyridine

To a solution of 2-amino-3-benzyloxypyridine (25.0 g, 124.9 mmol) and chloroacetaldehyde (50% wt in H₂O; 16.7 mL, 131.2 mmol) in 250 mL of EtOH was heated at reflux in a sealed tube for 19 h. Upon cooling to ambient temperature, the reaction mixture was concentrated and the resulting brown oil added 125 mL 1N NaOH then extracted with dichloromethane (DCM). The combined organic layers were washed with H₂O, dried over Na₂SO₄ and concentrated. Upon concentrating the solution, a tan solid formed which was filtered and dried to provide 25.8 g of crude product.

To a solution of crude 8-(benzyloxy)imidazo[1,2-a]pyridine (8.73 g, 38.9 mmol) in 100 mL of EtOH was added, dropwise, 4.8 mL (46.7 mmol) of a solution of 1:1 Br₂/H₂O at ambient temperature under an atmosphere of N₂. The resulting dark orange suspension was stirred at ambient temperature for 30 min, added 60 mL 1N NaOH, and the reaction mixture extracted with DCM. The combined organic layers were dried over Na₂SO₄ and concentrated. The crude product was purified by silica gel flash chromatography (eluted with 30% EtOAc/hexanes) to provide 7.04 g of product.

### 8-(Benzyloxy)-3-((trimethylsilyl)ethynyl)imidazo[1,2-a]pyridine

A mixture of 8-(benzyloxy)-3-bromoimidazo[1,2-a]pyridine (10.0 g, 33.0 mmol), 9.39 mL (66.0 mmol) of ethynyltrimethylsilane, 0.580 g (0.825 mmol) of Pd(PPh₃)₂Cl₂, 0.230 g (1.19 mmol) of CuI, and 5.09 mL (36.3 mmol) of diisopropylamine in 100 mL of acetonitrile was heated at reflux for 3 h under an atmosphere of N₂. Upon cooling to ambient temperature, the reaction mixture was concentrated and the crude product was purified by silica gel flash chromatography (eluted with 20-50% EtOAc/hexanes) to provide 6.74 g of product: 321 m/z (M+H).

### 3-((Trimethylsilyl)ethynyl)imidazo[1,2-a]pyridin-8-yl trifluoromethanesulfonate

To a cooled (0° C.) solution of 8-(benzyloxy)-3-((trimethylsilyl)ethynyl)imidazo[1,2-a]pyridine (3.44 g, 10.7 mmol) in 400 mL of DCM, under an atmosphere of N₂, was added via cannulation 100 mL (100 mmol) of boron trichloride (1.0M solution in hexanes). The reaction solution was stirred at 0° C./N₂ for 30 min, to which was added (0° C.) 200 mL H₂O followed by extraction with DCM. The combined organic layers were washed with brine, dried over Na₂SO₄ and concentrated. The crude product was purified by silica gel flash chromatography (eluted with 30% EtOAc/hexanes then 10% MeOH/DCM) to provide 2.32 g of deprotected product: 231 m/z (M+H).

To a cooled (−78° C.) solution of 8-(hydroxy)-3-((trimethylsilyl)ethynyl)imidazo[1,2-a]pyridine (2.32 g, 10.1 mmol) and 1.63 mL (20.1 mmol) of pyridine in 50 mL of DCM, under an atmosphere of N₂, was added 2.03 mL (12.1 mmol) of trifluoromethanesulfonic anhydride via syringe. Upon removing the cooling bath, the reaction solution was stirred at ambient temperature (N₂) for 2 h. The reaction mixture was poured into a stirring solution of 100 mL 1.0N HCl, the layers separated, and the organic layer washed successively with 1.0N HCl, H₂O, saturated aqueous NaHCO₃, and brine. The organic layer was dried over Na₂SO₄ and concentrated. The crude product was filtered through a small plug of silica gel (eluted with 30% EtOAc/hexanes), concentrated, and further dried in vacuo to provide 3.63 g of product: 363 m/z (M+H).

### N-(4-(Methylsulfonyl)phenyl)-3-((trimethylsilyl)ethynyl)imidazo[1,2-a]pyridin-8-amine

A mixture of 3-((trimethylsilyl)ethynyl)imidazo[1,2-a]pyridin-8-yl trifluoromethanesulfonate (0.329 g, 0.91 mmol), 0.186 (1.09 mmol) of 4-(methylsulfonyl)aniline, 0.083 g (0.091 mmol) of Pd₂(dba)₂, 0.087 g (0.181 mmol) of 2-dicyclohexylphosphino-2′,4′,6′-triisopropylbiphenyl, and 0.385 g (1.81 mmol) of potassium phosphate in 8 mL of DME was heated at 80° C. in a sealed tube overnight under an atmosphere of N₂. Upon cooling to ambient temperature, the reaction mixture was concentrated and the crude product was purified by silica gel flash chromatography (triethylamine-treated silica gel; eluted with 0-80% EtOAc/hexanes) to provide 0.058 g of product: 384 m/z (M+H).

### 3-Ethynyl-N-(4-(methylsulfonyl)phenyl)imidazo[1,2-a]pyridin-8-amine

To a solution of N-(4-(methylsulfonyl)phenyl)-3-((trimethylsilyl)ethynyl)imidazo[1,2-a]pyridin-8-amine (0.058 g, 0.15 mmol) in 1.5 mL of THF was added 0.23 mL (0.23 mmol) of tetrabutylammonium fluoride (1.0M in THF) at ambient temperature. The solution was stirred for 15 min, concentrated, and the crude product purified by silica gel flash chromatography (triethylamine-treated silica gel; eluted with 100% DCM then 5% MeOH/DCM) to provide a quantitative yield (0.047 g) of product: 312 m/z (M+H).

### 4-Methyl-3-O-(4-(methylsulfonyl)phenylamino)imidazo[1,2-a]pyridin-3-yl)ethynyl)-N-(4-(trifluoromethyl)pyridin-2-yl)benzamide

A mixture of 3-ethynyl-N-(4-(methylsulfonyl)phenyl)imidazo[1,2-a]pyridin-8-amine 5 (0.048 g, 0.154 mmol), 0.069 g (0.170 mmol) of 3-iodo-4-methyl-N-(4-(trifluoromethyl)pyridin-2-yl)benzamide, 0.009 g (0.008 mmol) of Pd(PPh₃)₄, 0.002 g (0.012 mmol) of CuI, and 0.04 mL (0.23 mmol) of diisopropylethylamine in 0.8 mL of DMF was stirred at ambient temperature overnight under an atmosphere of N₂. The reaction mixture was concentrated and the crude product was purified by silica gel flash chromatography (triethylamine-treated silica gel; eluted with 10% EtOAc/hexanes to 100% EtOAc) to provide 0.047 g of product as a solid: 590 m/z (M+H).

### Example 13

### 4-methyl-3-((8-(4-sulfamoylphenylamino)imidazo[1,2-a]pyridin-3-yl)ethynyl)-N-(4-(trifluoromethyl)pyridin-2-yl)benzamide

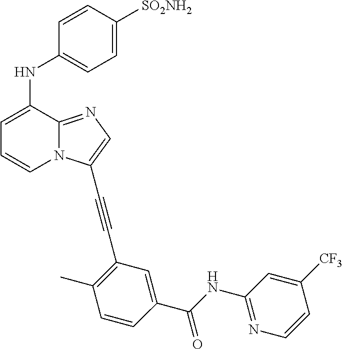 [SMILES: Cc1ccc(C(=O)Nc2cc(C(F)(F)F)ccn2)cc1C#Cc1cnc2c(Nc3ccc(S(N)(=O)=O)cc3)cccn12]

The title compound was synthesized from 3-ethynyl-N-(4-sulfamoylphenyl)imidazo[1,2-a]pyridin-8-amine and 3-iodo-4-methyl-N-(4-(trifluoromethyl)pyridin-2-yl)benzamide in a manner similar to that described for Example 12. The product was obtained as a solid: 591 m/z (M+H).

### Example 14

### (R)—N-(4-((3-(Dimethylamino)pyrrolidin-1-yl)methyl)-3-(trifluoromethyl)phenyl)-3-(imidazo[1,2-b]pyridazin-3-ylethynyl)-4-methylbenzamide

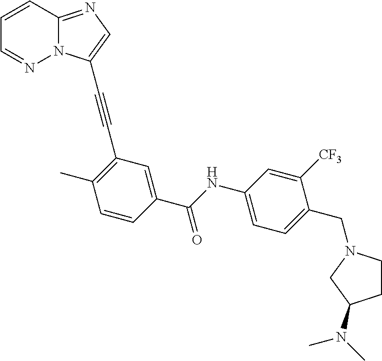 [SMILES: Cc1ccc(C(=O)Nc2ccc(CN3CC[C@@H](N(C)C)C3)c(C(F)(F)F)c2)cc1C#Cc1cnc2cccnn12]

### 3-((Trimethylsilyl)ethynyl)imidazo[1,2-b]pyridazine

A mixture of 3-bromoimidazo[1,2-b]pyridazine (36.78 g, 0.186 mol; prepared according to Stanovnik, B. et al. Synthesis (1981), 12, 987-989), ethynyltrimethylsilane (21.89 g, 0.223 mol), Pd(PPh₃)₄ (10.73 g, 9.29 mmol), CuI (5.30 g, 0.028 mol), and diisopropylethylamine (32.4 mL, 0.279 mol) in 150 mL of DMF was stirred at ambient temperature, under an atmosphere of N₂, for 1 h. The reaction mixture was concentrated and the crude product was purified by silica gel flash chromatography (eluted with 0-5% MeOH/DCM) to provide 28.46 g of product.

### 3-Ethynylimidazo[1,2-b]pyridazine

To a solution of 3-((trimethylsilyl)ethynyl) imidazo[1,2-b]pyridazine (28.46 g, 0.132 mol) in 200 mL of THF was added 145 mL (0.145 mol) of tetrabutylammonium fluoride (1.0M in THF) at ambient temperature. The solution was stirred for 15 min, concentrated, and the crude product purified by silica gel flash chromatography (eluted with 0-5% MeOH/DCM) to provide 17.84 g of product.

### 1-(Bromomethyl)-4-nitro-2-(trifluoromethyl)benzene

A suspension of 2-methyl-5-nitrobenzotrifluoride (3.90 g, 19 mmol), N-bromosuccinimide (NBS, 3.56 g, 20 mmol), and 2,2′-azobis(2-methylpropionitrile) (AIBN, 0.094 g, 0.6 mmol) in 40 mL of CCl₄ was heated at reflux under N₂ for 16 h. HPLC indicated ca. 50% conversion. Additional NBS (10 mmol) and AIBN (0.6 mmol) were added and the mixture was heated at reflux for another 14 h. HPLC indicated ca. 80% conversion. The reaction mixture was cooled to ambient temperature, and the solid was filtered and washed with EtOAc. The combined filtrate was washed with aq. NaHCO₃, dried over Na₂SO₄, filtered, concentrated on rotovap, and further dried under vacuum. ¹H NMR indicated the ratio of desired product to unreacted 2-methyl-5-nitrobenzotrifluoride to be 75:25. This material was used directly in the next step.

### (R)—N,N-Dimethyl-1-(4-nitro-2-(trifluoromethyl)benzyl)pyrrolidin-3-amine

To a solution of crude 1-(bromomethyl)-4-nitro-2-(trifluoromethyl)benzene (17.5 mmol, 75% pure) in 40 mL of DCM was added Et₃N (2.69 mL, 19.3 mmol) and (R)-(+)-3-(dimethylamino)pyrrolidine (2.0 g, 17.5 mmol). After stirring overnight at ambient temperature under an atmosphere of N₂, the reaction solution was concentrated, added aq. NaHCO₃ (100 mL), and the resulting mixture extracted with DCM (4×50 mL). The combined organic layer was dried over Na₂SO₄, filtered, concentrated, and the resulting residue was purified by silica gel chromatography (eluted with 0-10% MeOH/DCM) to provide 3.35 g of product as a yellow oil.

### (R)-1-(4-Amino-2-(trifluoromethyl)benzyl)-N,N-dimethylpyrrolidin-3-amine

To a solution of (R)—N,N-dimethyl-1-(4-nitro-2-(trifluoromethyl)benzyl)pyrrolidin-3-amine (1.20 g, 3.79 mmol) in 20 mL of wet EtOH was added 0.26 g of Pd/C (10% Pd on C) and the mixture shaken in a Parr apparatus (pressure reaction vessel purged thoroughly with H₂ and pressure regulated at 45 psi throughout) for 2-3 h. The reaction mixture was filtered through a small pad of celite, washed with EtOAc, and the combined organics concentrated to provide a quantitative yield of a light yellow oil. This material was used directly in the next step.

### (R)—N-(4-((3-(Dimethylamino)pyrrolidin-1-yl)methyl)-3-(trifluoromethyl)phenyl)-3-iodo-4-methylbenzamide

To a cooled (0° C.) solution of (R)-1-(4-amino-2-(trifluoromethyl)benzyl)-N,N-dimethylpyrrolidin-3-amine (3.79 mmol) in 14 mL DCM, under an atmosphere of N₂, was added 3-Iodo-4-methylbenzoyl chloride (1.17 g, 4.17 mmol; CAS#52107-98-9, prepared from the reaction of 3-iodo-4-methylbenzoic acid and SOCl₂) followed by dropwise addition of N,N-diisopropylethylamine (2.64 mL, 15.2 mmol). After stirring to ambient temperature over 1.5 h, the reaction mixture was concentrated and the crude product was purified by silica gel chromatography (eluted with 0-8% MeOH/DCM; MeOH was pre-saturated with ammonia gas), to provide 0.71 g of product as a thick yellow oil.

### (R)—N-(4-O-(dimethylamino)pyrrolidin-1-yl)methyl)-3-(trifluoromethyl)phenyl)-3-(imidazo[1,2-b]pyridazin-3-ylethynyl)-4-methylbenzamide

A mixture of 3-ethynylimidazo[1,2-b]pyridazine (0.051 g, 0.34 mmol), 0.150 g (0.28 mmol) of (R)—N-(4-((3-(dimethylamino)pyrrolidin-1-yl)methyl)-3-(trifluoromethyl)phenyl)-3-iodo-4-methylbenzamide, 0.016 g (0.014 mmol) of Pd(PPh₃)₄, 0.004 g (0.021 mmol) of CuI, and 0.09 mL (0.51 mmol) of N,N-diisopropylethylamine in 3.5 mL of DMF was stirred at ambient temperature, under an atmosphere of N₂, for 3 days (reaction pushed to completion with additional equivalents of reagents and heating to 80° C.). The reaction mixture was concentrated and the crude product was purified by silica gel chromatography (eluted with 0-10% MeOH/DCM; MeOH was pre-saturated with ammonia gas) to provide 0.020 g of product as a solid: 547 m/z (M+H).

### Alternative Synthesis of (R)—N-(4-((3-(Dimethylamino)pyrrolidin-1-yl)methyl)-3-(trifluoromethyl)phenyl)-3-(imidazo[1,2-b]pyridazin-3-ylethynyl)-4-methylbenzamide

(R)—N-(4-((3-(Dimethylamino)pyrrolidin-1-yl)methyl)-3-(trifluoromethyl)phenyl)-3-(imidazo[1,2-b]pyridazin-3-ylethynyl)-4-methylbenzamide and its mono hydrochloride salt can be prepared in an alternative synthesis similar to that described in Example 1 from 3-(imidazo[1,2-b]pyridazin-3-ylethynyl)-4-methylbenzoic acid and (R)-1-(4-Amino-2-(trifluoromethyl)benzyl)-N,N-dimethylpyrrolidin-3-amine (as prepared above). The 3-(imidazo[1,2-b]pyridazin-3-ylethynyl)-4-methylbenzoic acid is prepared in a manner similar to that described in Example 1 using 3-Ethynylimidazo[1,2-b]pyridazine and 3-iodo-4-methylbenzoic acid as Sonogashira coupling partners.

### Example 15

### N-(3-(Imidazo[1,2-b]pyridazin-3-ylethynyl)-4-methylphenyl)-4-((4-methylpiperazin-1-yl)methyl)-3-(trifluoromethyl)benzamide

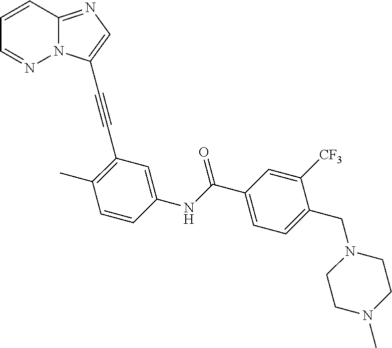 [SMILES: Cc1ccc(NC(=O)c2ccc(CN3CCN(C)CC3)c(C(F)(F)F)c2)cc1C#Cc1cnc2cccnn12]

The title compound was synthesized from 3-ethynylimidazo[1,2-b]pyridazine and N-(3-iodo-4-methylphenyl)-4-((4-methylpiperazin-1-yl)methyl)-3-(trifluoromethyl)benzamide in a manner similar to that described for Example 14. The product was obtained as a solid: 533 m/z (M+H).

### N-(3-Iodo-4-methylphenyl)-4-((4-methylpiperazin-1-yl)methyl)-3-(trifluoromethyl)benzamide

To a flask containing 1.0 g (2.67 mmol) of 4-[(4-methyl-1-piperazinyl)methyl]-3-(trifluoromethyl)-benzoic acid (CAS#859027-02-4; prepared according to Asaki, T. et al. Bioorg. Med. Chem. Lett. (2006), 16, 1421-1425), 0.62 g (2.67 mmol) of 3-Iodo-4-methylaniline, 0.77 g (4.0 mmol) of N-(3-dimethylaminopropyl)-N′-ethylcarbodiimide hydrochloride (EDAC), and 0.43 g (3.2 mmol) of N-hydroxybenzotriazole monohydrate (HOBt.H₂O) was added 5 mL of DCM and 5 mL of triethylamine. The solution was stirred at ambient temperature under an atmosphere of N₂ for 3 days, concentrated, and the crude product purified by silica gel chromatography (eluted with 100% EtOAc then 10% MeOH/EtOAc), to provide 0.69 g of product as a white solid.

### Example 16

### 3-(Imidazo[1,2-b]pyridazin-3-ylethynyl)-4-methyl-N-(4-((4-methylpiperazin-1-yl)methyl)-3-(trifluoromethyl)phenyl)benzamide

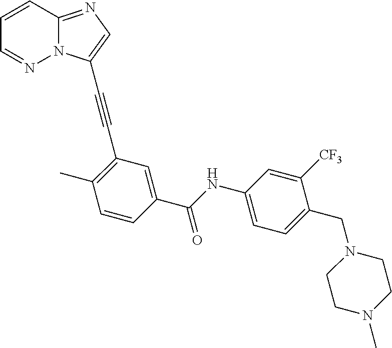 [SMILES: Cc1ccc(C(=O)Nc2ccc(CN3CCN(C)CC3)c(C(F)(F)F)c2)cc1C#Cc1cnc2cccnn12]

The title compound was synthesized in a manner similar to that described for Example 14, from 3-ethynylimidazo[1,2-b]pyridazine and 3-iodo-4-methyl-N-(4-((4-methylpiperazin-1-yl)methyl)-3-(trifluoromethyl)phenyl)benzamide (Prepared as described in Example 2). The product was obtained as a solid: 533 m/z (M+H).

### Alternative Synthesis of 3-(Imidazo[1,2-b]pyridazin-3-ylethynyl)-4-methyl-N-(4-((4-methylpiperazin-1-yl)methyl)-3-(trifluoromethyl)phenyl)benzamide

3-(Imidazo[1,2-b]pyridazin-3-ylethynyl)-4-methyl-N-(4-((4-methylpiperazin-1-yl)methyl)-3-(trifluoromethyl)phenyl)benzamide and its mono hydrochloride salt can be prepared in an alternative synthesis similar to that described in Example 1 from 3-(imidazo[1,2-b]pyridazin-3-ylethynyl)-4-methylbenzoic acid and 4-((4-methylpiperazin-1-yl)methyl)-3-(trifluoromethyl)aniline (as prepared in example 2). The 3-(imidazo[1,2-b]pyridazin-3-ylethynyl)-4-methylbenzoic acid is prepared in a manner similar to that described in Example 1 using 3-Ethynylimidazo[1,2-b]pyridazine and 3-iodo-4-methylbenzoic acid as Sonogashira coupling partners.

### Example 17

### N-(3-Chloro-4-((4-methylpiperazin-1-yl)methyl)phenyl)-3-(imidazo[1,2-b]pyridazin-3-ylethynyl)-4-methylbenzamide

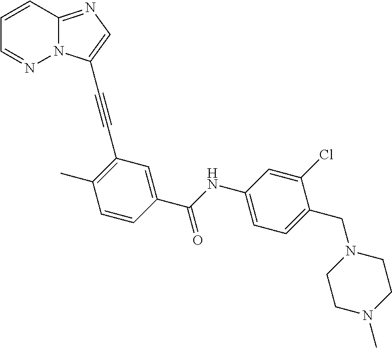 [SMILES: Cc1ccc(C(=O)Nc2ccc(CN3CCN(C)CC3)c(Cl)c2)cc1C#Cc1cnc2cccnn12]

The title compound was synthesized according to Example 14, from 3-ethynylimidazo[1,2-b]pyridazine and N-(3-chloro-4-((4-methylpiperazin-1-yl)methyl)phenyl)-3-iodo-4-methylbenzamide. The product was obtained as a solid: 499 m/z (M+H).

### 1-(Bromomethyl)-2-chloro-4-nitro-benzene

A suspension of 2-chloro-4-nitrotoluene (10.0 g, 58.3 mmol), N-bromosuccinimide (NBS, 10.9 g, 61.2 mmol), and 2,2′-azobis(2-methylpropionitrile) (AIBN, 0.29 g, 1.75 mmol) in 120 mL of CCl₄ was heated at reflux under an atmosphere of N₂ for 12 h. The reaction mixture was cooled to ambient temperature, and the solid was filtered and washed with EtOAc. The combined filtrate was washed with aq. NaHCO₃, dried over Na₂SO₄, filtered, concentrated on rotovap, and further dried under vacuum. ¹H NMR indicated the ratio of desired product to unreacted 2-chloro-4-nitrotoluene to be 50:50. This material was used directly in the next step.

### 1-(2-Chloro-4-nitrobenzyl)-4-methylpiperazine

To a solution of crude 1-(bromomethyl)-2-chloro-4-nitro-benzene (29.1 mmol; 50% pure) in 30 mL of DCM was added Et₃N (4.2 mL, 30 mmol) and 1-methylpiperazine (3.4 mL, 30 mmol). After stirring for 3 h at ambient temperature, aq. NaHCO₃ was added and the mixture was extracted with DCM. The combined organic layer was dried over Na₂SO₄, filtered, concentrated, and the resulting residue was purified by silica gel chromatography (eluted with 5% MeOH/DCM) to provide 6.80 g of product as a dark yellow oil.

### 3-Chloro-4-((4-methylpiperazin-1-yl)methyl)aniline

To a solution of 1-(2-chloro-4-nitrobenzyl)-4-methylpiperazine (0.96 g, 3.6 mmol) in MeOH/water (4:1, 50 mL) was added 1.80 g (33.7 mmol) of NH₄Cl and 1.47 g (26.3 mmol) of Fe dust and the mixture heated at reflux under an atmosphere of N₂ for 2 h (HPLC indicated no progress). To this was added 4 mL of glacial acetic acid and the mixture heated at reflux for an additional 2 h. The reaction mixture was cooled to ambient temperature, filtered, and the filtrate concentrated. The residue was partitioned between EtOAc and saturated aq. NaHCO₃, the separated aqueous layer was extracted with EtOAc, and the combined organics washed with brine and dried over Na₂SO₄. Upon concentration, the crude product was purified by silica gel chromatography (eluted with 5-7% MeOH/DCM; silica gel deactivated with 1% triethylamine/DCM) to provide 0.53 g of product.

### Alternative Synthesis of N-(3-Chloro-4-((4-methylpiperazin-1-yl)methyl)phenyl)-3-(imidazo[1,2-b]pyridazin-3-ylethynyl)-4-methylbenzamide

N-(3-Chloro-4-((4-methyl piperazin-1-yl)methyl)phenyl)-3-(imidazo[1,2-b]pyridazin-3-ylethynyl)-4-methylbenzamide and its mono hydrochloride salt can be prepared in an alternative synthesis similar to that described in Example 1 from 3-(imidazo[1,2-b]pyridazin-3-ylethynyl)-4-methylbenzoic acid and 3-Chloro-4-((4-methylpiperazin-1-yl)methyl)aniline (as prepared above). The 3-(imidazo[1,2-b]pyridazin-3-ylethynyl)-4-methylbenzoic acid is prepared in a manner similar to that described in Example 1 using 3-Ethynylimidazo[1,2-b]pyridazine and 3-iodo-4-methylbenzoic acid as Sonogashira coupling partners.

### Example 18

### N-(3-Cyclopropyl-4-((4-methylpiperazin-1-yl)methyl)phenyl)-3-(imidazo[1,2-b]pyridazin-3-ylethynyl)-4-methylbenzamide

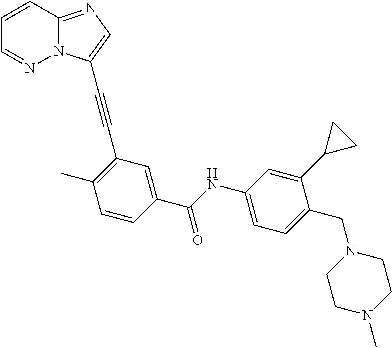 [SMILES: Cc1ccc(C(=O)Nc2ccc(CN3CCN(C)CC3)c(C3CC3)c2)cc1C#Cc1cnc2cccnn12]

The title compound was synthesized from 3-ethynylimidazo[1,2-b]pyridazine and N-(3-cyclopropyl-4-((4-methylpiperazin-1-yl)methyl)phenyl)-3-iodo-4-methylbenzamide in a manner similar to that described for Example 14 (nitro reduction performed in a manner similar to that described for Example 17; 0.25M in MeOH/10% AcOH). The product was obtained as a solid: 505 m/z (M+H).

### 1-(2-Cyclopropyl-4-nitrobenzyl)-4-methylpiperazine

A mixture of 1-(2-bromo-4-nitrobenzyl)-4-methylpiperazine (0.94 g, 3.0 mmol), 0.77 g (9.0 mmol) of cyclopropylboronic acid, 0.067 g (0.30 mmol) of Pd(OAc)₂, 2.87 g (13.5 mmol) of K₃PO₄, and 0.168 g (0.60 mmol) of tricyclohexylphosphine in 18 mL of toluene/water (5:1) was heated at reflux under an atmosphere of N₂ for 19 h. The reaction mixture was concentrated and the crude product was purified by silica gel chromatography (eluted with 5% MeOH/DCM; MeOH was pre-saturated with ammonia gas) to provide 0.80 g of product.

### Example 19

### 3-(Imidazo[1,2-b]pyridazin-3-ylethynyl)-N-(4-((4-methylpiperazin-1-yl)methyl)-3-(trifluoromethyl)phenyl)benzamide

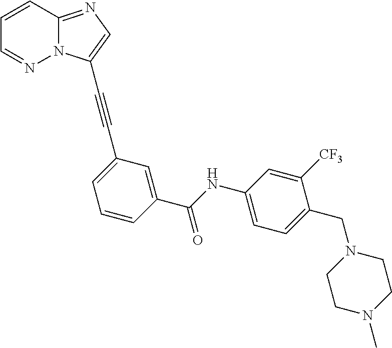 [SMILES: CN1CCN(Cc2ccc(NC(=O)c3cccc(C#Cc4cnc5cccnn45)c3)cc2C(F)(F)F)CC1]

The title compound was synthesized from 3-ethynylimidazo[1,2-b]pyridazine and 3-iodo-N-(4-((4-methylpiperazin-1-yl)methyl)-3-(trifluoromethyl)phenyl)benzamide in a manner similar to that described for Example 14. The product was obtained as a solid: 519 m/z (M+H).

The titled compound can also be prepared according to the alternative synthesis described in example 1 from 3-(imidazo[1,2-b]pyridazin-3-ylethynyl)-4-methylbenzoic acid and 4-((4-methylpiperazin-1-yl)methyl)-3-(trifluoromethyl)aniline (as prepared in example 2). The 3-(imidazo[1,2-b]pyridazin-3-ylethynyl)-4-methylbenzoic acid is prepared in a manner similar to that described in Example 1 using 3-Ethynylimidazo[1,2-b]pyridazine and 3-iodo-4-methylbenzoic acid as Sonogashira coupling partners.

### Example 20

### N-(4-((4-(2-Hydroxyethyl)piperazin-1-yl)methyl)-3-(trifluoromethyl)phenyl)-3-(imidazo[1,2-b]pyridazin-3-ylethynyl)-4-methylbenzamide

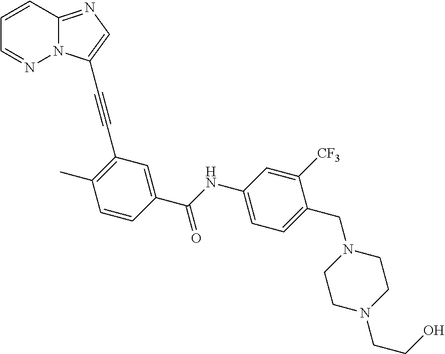 [SMILES: Cc1ccc(C(=O)Nc2ccc(CN3CCN(CCO)CC3)c(C(F)(F)F)c2)cc1C#Cc1cnc2cccnn12]

The title compound was synthesized from 3-ethynylimidazo[1,2-b]pyridazine and N-(4-((4-(2-hydroxyethyl)piperazin-1-yl)methyl)-3-(trifluoromethyl)phenyl)-3-iodo-4-methylbenzamide in a manner similar to that described for Example 14. The product was obtained as a solid: 563 m/z (M+H).

### Example 21

### 3-(Imidazo[1,2-b]pyridazin-3-ylethynyl)-4-methyl-N-(4-(piperazin-1-ylmethyl)-3-(trifluoromethyl)phenyl)benzamide

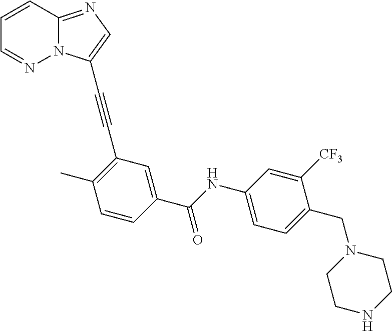 [SMILES: Cc1ccc(C(=O)Nc2ccc(CN3CCNCC3)c(C(F)(F)F)c2)cc1C#Cc1cnc2cccnn12]

The title compound was synthesized from 3-ethynylimidazo[1,2-b]pyridazine and tert-butyl-4-(4-(3-iodo-4-methylbenzamido)-2-(trifluoromethyl)benzyl)piperazine-1-carboxylate in a manner similar to that described for Example 14. Following deprotection using saturated MeOH/HCl (g), the product was obtained as a tris HCl salt: 519 m/z (M+H).

### Representative Biological Data

Compounds of this invention are evaluated in a variety of assays to determine their biological activities. For example, the compounds of the invention can be tested for their ability to inhibit various protein kinases of interest. Some of the compounds tested displayed potent nanomolar activity against the following kinases: Abl, Abl T315I, Src, PDGFR, c-kit, and FGFR. Furthermore, several of these compounds were screened for antiproliferative activity in BaF3 cells transfected with either wild-type Bcr-Abl or the Bcr-Abl T3151 mutant and demonstrated activity in the range of 1-100 nM.

The compounds can also be evaluated for their cytotoxic or growth inhibitory effects on tumor cells of interest, e.g., as described in more detail below and as shown above for some representative compounds. See e.g., WO 03/000188, pages 115-136, the full contents of which are incorporated herein by reference.

Some representative compounds are depicted below.

| | T315I |
|---|---|
| | cell |
| | prolifer- |
| | ation |
| Compounds of the Invention | (nM) |
| 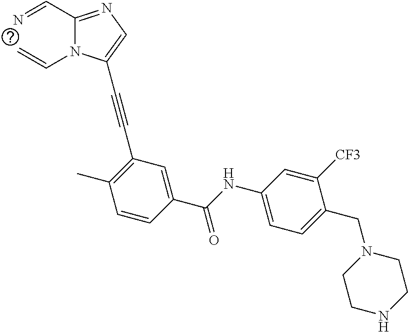 [SMILES: C=Cn1c(C#Cc2cc(C(=O)Nc3ccc(CN4CCNCC4)c(C(F)(F)F)c3)ccc2C)cnc1C=N] | <1000 |
| 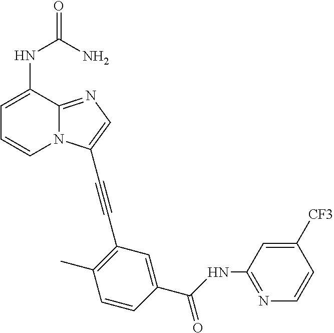 [SMILES: Cc1ccc(C(=O)Nc2cc(C(F)(F)F)ccn2)cc1C#Cc1cnc2c(NC(N)=O)cccn12] | <1000 |
| 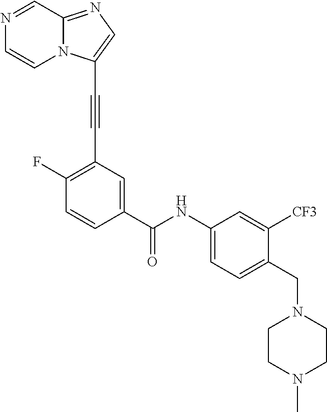 [SMILES: CN1CCN(Cc2ccc(NC(=O)c3ccc(F)c(C#Cc4cnc5cnccn45)c3)cc2C(F)(F)F)CC1] | <1000 |
| 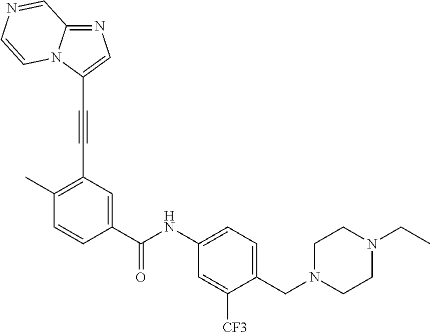 [SMILES: CCN1CCN(Cc2ccc(NC(=O)c3ccc(C)c(C#Cc4cnc5cnccn45)c3)cc2C(F)(F)F)CC1] | <1000 |
| 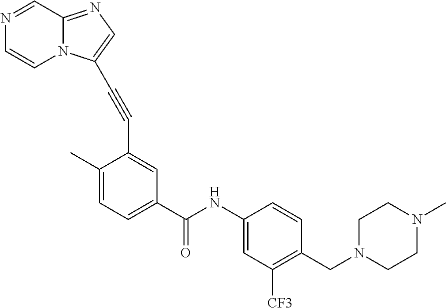 [SMILES: Cc1ccc(C(=O)Nc2ccc(CN3CCN(C)CC3)c(C(F)(F)F)c2)cc1C#Cc1cnc2cnccn12] | <1000 |
| 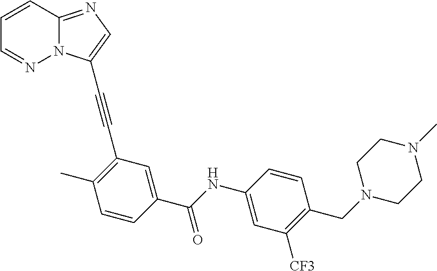 [SMILES: Cc1ccc(C(=O)Nc2ccc(CN3CCN(C)CC3)c(C(F)(F)F)c2)cc1C#Cc1cnc2cccnn12] | <1000 |
| 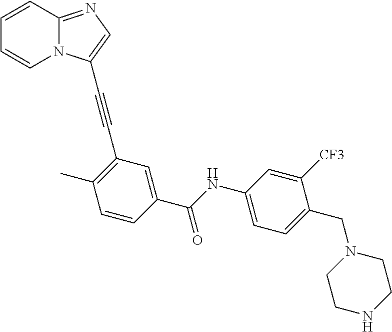 [SMILES: Cc1ccc(C(=O)Nc2ccc(CN3CCNCC3)c(C(F)(F)F)c2)cc1C#Cc1cnc2ccccn12] | <1000 |
| 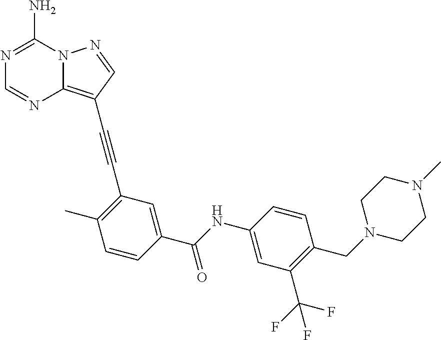 [SMILES: Cc1ccc(C(=O)Nc2ccc(CN3CCN(C)CC3)c(C(F)(F)F)c2)cc1C#Cc1cnn2c(N)ncnc12] | <1000 |
| 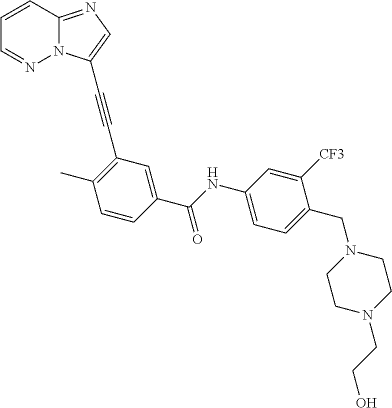 [SMILES: Cc1ccc(C(=O)Nc2ccc(CN3CCN(CCO)CC3)c(C(F)(F)F)c2)cc1C#Cc1cnc2cccnn12] | <1000 |
| 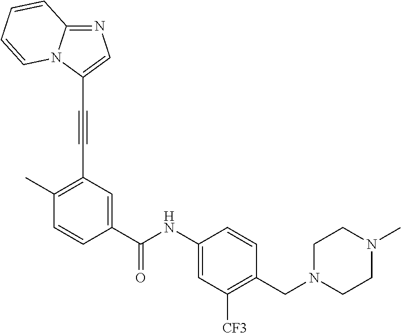 [SMILES: Cc1ccc(C(=O)Nc2ccc(CN3CCN(C)CC3)c(C(F)(F)F)c2)cc1C#Cc1cnc2ccccn12] | <1000 |
| 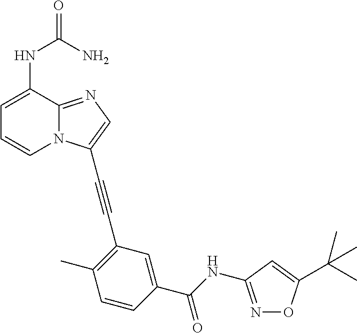 [SMILES: Cc1ccc(C(=O)Nc2cc(C(C)(C)C)on2)cc1C#Cc1cnc2c(NC(N)=O)cccn12] | <1000 |
| 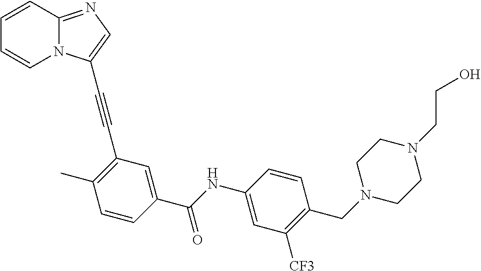 [SMILES: Cc1ccc(C(=O)Nc2ccc(CN3CCN(CCO)CC3)c(C(F)(F)F)c2)cc1C#Cc1cnc2ccccn12] | <1000 |
| 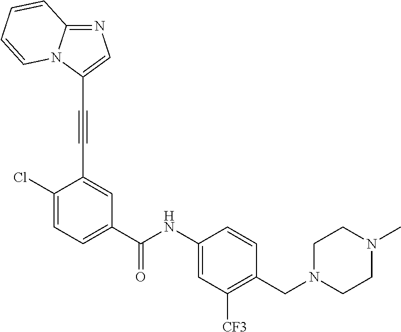 [SMILES: CN1CCN(Cc2ccc(NC(=O)c3ccc(Cl)c(C#Cc4cnc5ccccn45)c3)cc2C(F)(F)F)CC1] | <1000 |
| 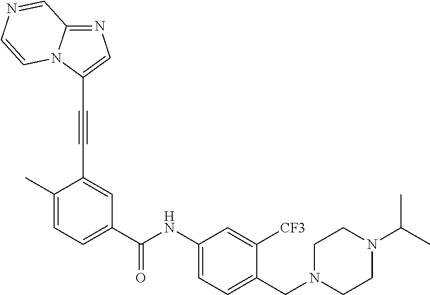 [SMILES: Cc1ccc(C(=O)Nc2ccc(CN3CCN(C(C)C)CC3)c(C(F)(F)F)c2)cc1C#Cc1cnc2cnccn12] | <1000 |
| 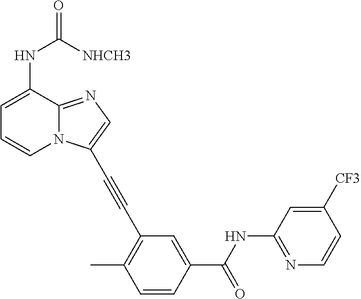 [SMILES: CNC(=O)Nc1cccn2c(C#Cc3cc(C(=O)Nc4cc(C(F)(F)F)ccn4)ccc3C)cnc12] | <1000 |
| 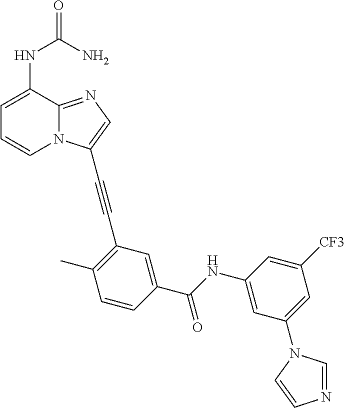 [SMILES: Cc1ccc(C(=O)Nc2cc(-n3ccnc3)cc(C(F)(F)F)c2)cc1C#Cc1cnc2c(NC(N)=O)cccn12] | <1000 |
| 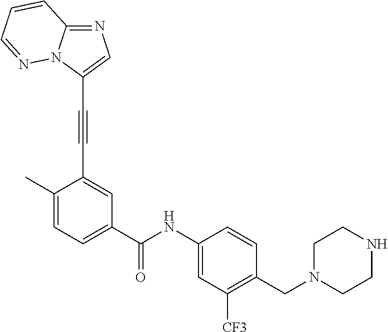 [SMILES: Cc1ccc(C(=O)Nc2ccc(CN3CCNCC3)c(C(F)(F)F)c2)cc1C#Cc1cnc2cccnn12] | <1000 |
| 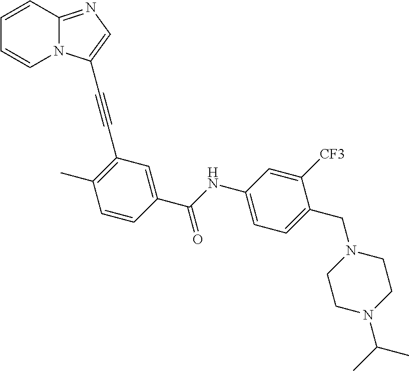 [SMILES: Cc1ccc(C(=O)Nc2ccc(CN3CCN(C(C)C)CC3)c(C(F)(F)F)c2)cc1C#Cc1cnc2ccccn12] | <1000 |
| 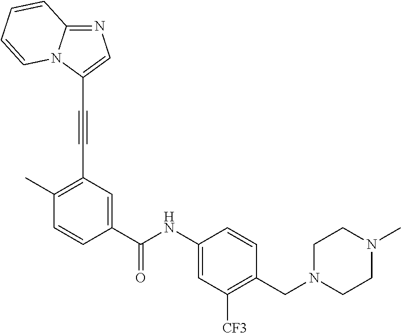 [SMILES: Cc1ccc(C(=O)Nc2ccc(CN3CCN(C)CC3)c(C(F)(F)F)c2)cc1C#Cc1cnc2ccccn12] | <1000 |
| 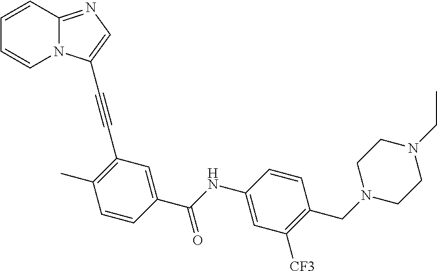 [SMILES: CCN1CCN(Cc2ccc(NC(=O)c3ccc(C)c(C#Cc4cnc5ccccn45)c3)cc2C(F)(F)F)CC1] | <1000 |
| 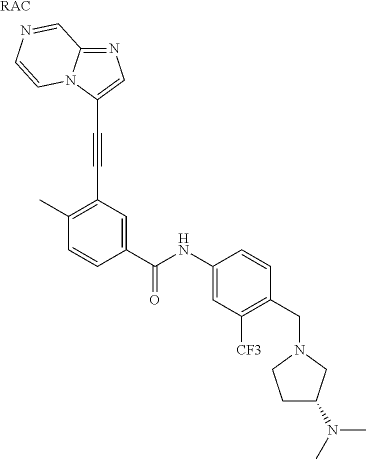 | <1000 |
| 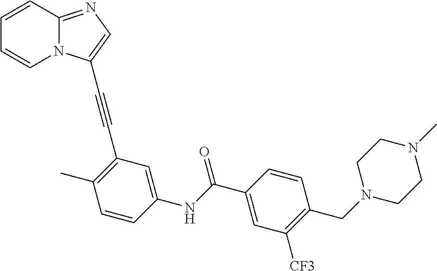 [SMILES: Cc1ccc(NC(=O)c2ccc(CN3CCN(C)CC3)c(C(F)(F)F)c2)cc1C#Cc1cnc2ccccn12] | <1000 |
| 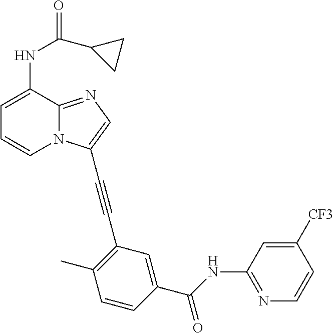 [SMILES: Cc1ccc(C(=O)Nc2cc(C(F)(F)F)ccn2)cc1C#Cc1cnc2c(NC(=O)C3CC3)cccn12] | <1000 |
| 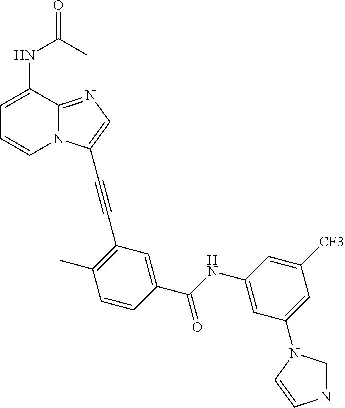 [SMILES: CC(=O)Nc1cccn2c(C#Cc3cc(C(=O)Nc4cc(N5C=CNC5)cc(C(F)(F)F)c4)ccc3C)cnc12] | <1000 |
| 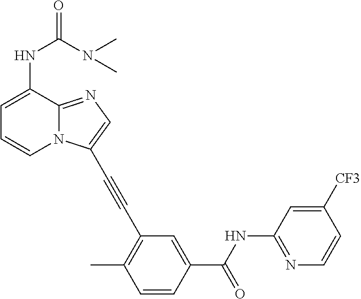 [SMILES: Cc1ccc(C(=O)Nc2cc(C(F)(F)F)ccn2)cc1C#Cc1cnc2c(NC(=O)N(C)C)cccn12] | <1000 |
| 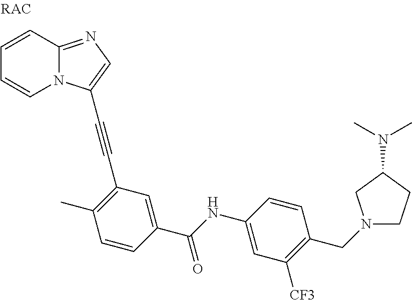 [SMILES: Cc1ccc(C(=O)Nc2ccc(CN3CC[C@@H](N(C)C)C3)c(C(F)(F)F)c2)cc1C#Cc1cnc2ccccn12] | <1000 |
| 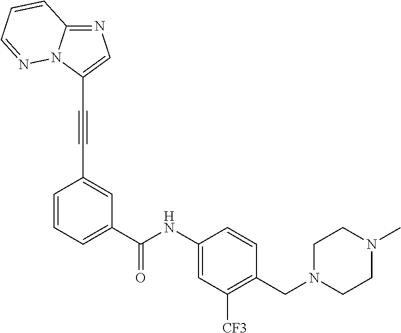 [SMILES: CN1CCN(Cc2ccc(NC(=O)c3cccc(C#Cc4cnc5cccnn45)c3)cc2C(F)(F)F)CC1] | <1000 |
| 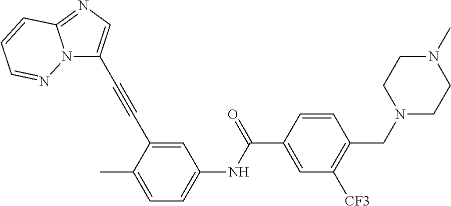 [SMILES: Cc1ccc(NC(=O)c2ccc(CN3CCN(C)CC3)c(C(F)(F)F)c2)cc1C#Cc1cnc2cccnn12] | <1000 |
| 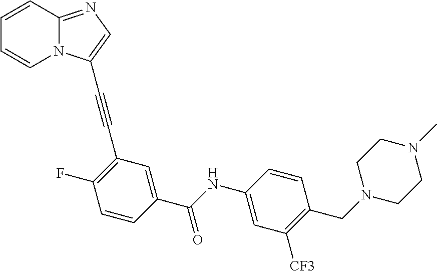 [SMILES: CN1CCN(Cc2ccc(NC(=O)c3ccc(F)c(C#Cc4cnc5ccccn45)c3)cc2C(F)(F)F)CC1] | <1000 |
| 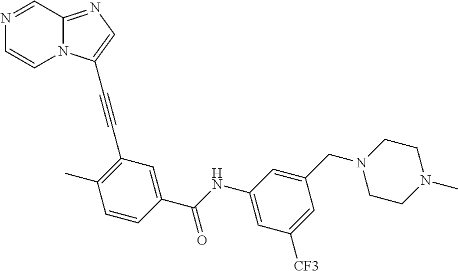 [SMILES: Cc1ccc(C(=O)Nc2cc(CN3CCN(C)CC3)cc(C(F)(F)F)c2)cc1C#Cc1cnc2cnccn12] | <1000 |
| 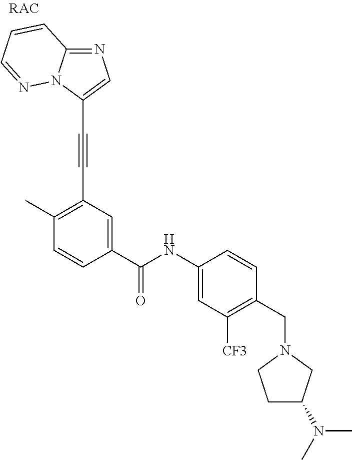 [SMILES: Cc1ccc(C(=O)Nc2ccc(CN3CC[C@@H](N(C)C)C3)c(C(F)(F)F)c2)cc1C#Cc1cnc2cccnn12] | <1000 |
| 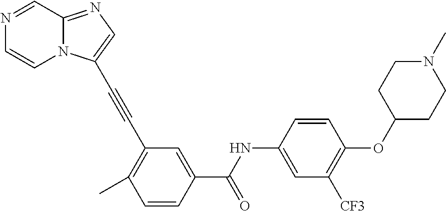 [SMILES: Cc1ccc(C(=O)Nc2ccc(OC3CCN(C)CC3)c(C(F)(F)F)c2)cc1C#Cc1cnc2cnccn12] | <1000 |
| 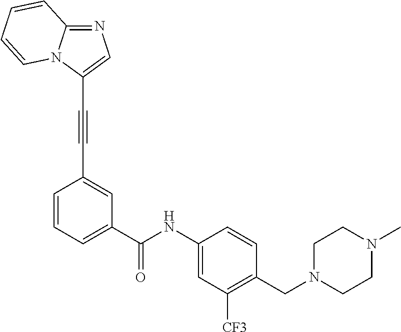 [SMILES: CN1CCN(Cc2ccc(NC(=O)c3cccc(C#Cc4cnc5ccccn45)c3)cc2C(F)(F)F)CC1] | <1000 |
| 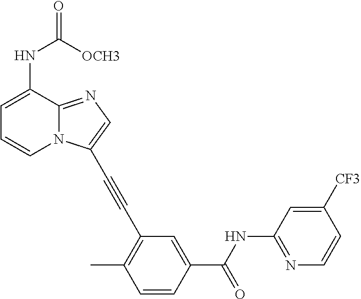 [SMILES: COC(=O)Nc1cccn2c(C#Cc3cc(C(=O)Nc4cc(C(F)(F)F)ccn4)ccc3C)cnc12] | <1000 |
| 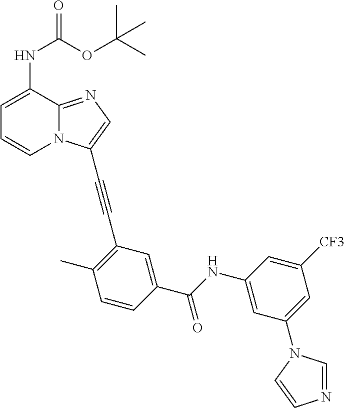 [SMILES: Cc1ccc(C(=O)Nc2cc(-n3ccnc3)cc(C(F)(F)F)c2)cc1C#Cc1cnc2c(NC(=O)OC(C)(C)C)cccn12] | <1000 |
| 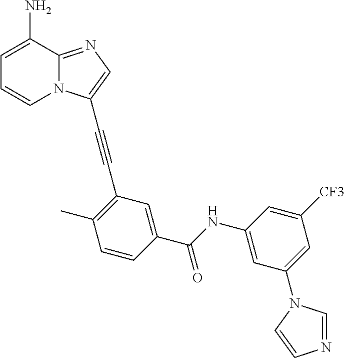 [SMILES: Cc1ccc(C(=O)Nc2cc(-n3ccnc3)cc(C(F)(F)F)c2)cc1C#Cc1cnc2c(N)cccn12] | <1000 |
| 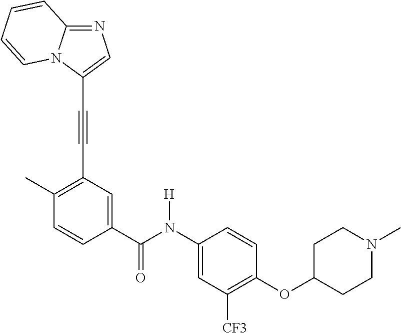 [SMILES: Cc1ccc(C(=O)Nc2ccc(OC3CCN(C)CC3)c(C(F)(F)F)c2)cc1C#Cc1cnc2ccccn12] | <1000 |
| 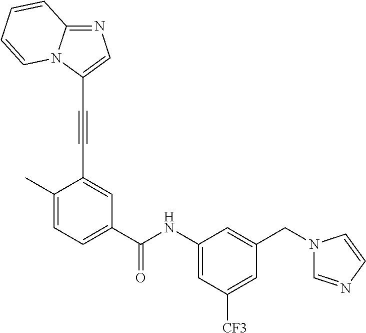 [SMILES: Cc1ccc(C(=O)Nc2cc(Cn3ccnc3)cc(C(F)(F)F)c2)cc1C#Cc1cnc2ccccn12] | <1000 |
| 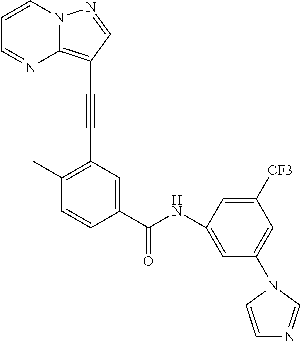 [SMILES: Cc1ccc(C(=O)Nc2cc(-n3ccnc3)cc(C(F)(F)F)c2)cc1C#Cc1cnn2cccnc12] | <1000 |
| 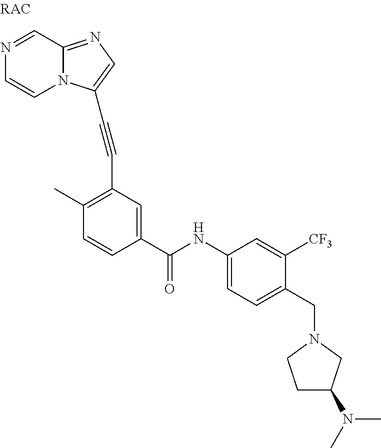 [SMILES: Cc1ccc(C(=O)Nc2ccc(CN3CC[C@H](N(C)C)C3)c(C(F)(F)F)c2)cc1C#Cc1cnc2cnccn12] | <1000 |
| 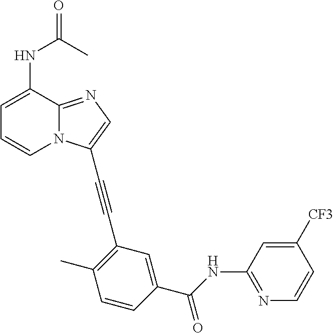 [SMILES: CC(=O)Nc1cccn2c(C#Cc3cc(C(=O)Nc4cc(C(F)(F)F)ccn4)ccc3C)cnc12] | <1000 |
| 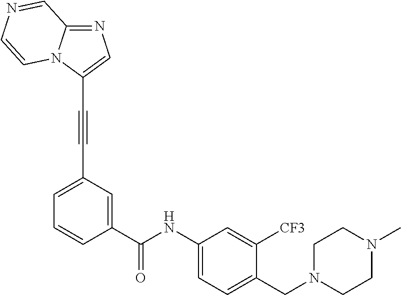 [SMILES: CN1CCN(Cc2ccc(NC(=O)c3cccc(C#Cc4cnc5cnccn45)c3)cc2C(F)(F)F)CC1] | <1000 |
| 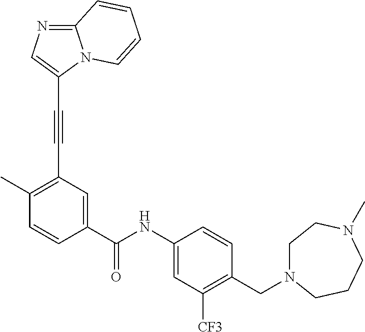 | <1000 |
| 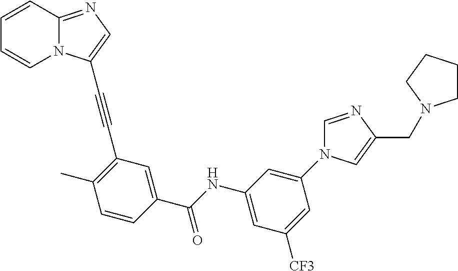 [SMILES: Cc1ccc(C(=O)Nc2cc(-n3cnc(CN4CCCC4)c3)cc(C(F)(F)F)c2)cc1C#Cc1cnc2ccccn12] | <1000 |
| 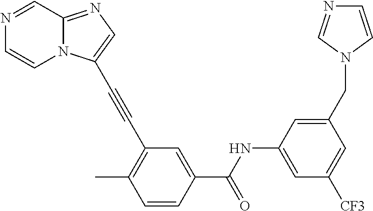 [SMILES: Cc1ccc(C(=O)Nc2cc(Cn3ccnc3)cc(C(F)(F)F)c2)cc1C#Cc1cnc2cnccn12] | <1000 |
| 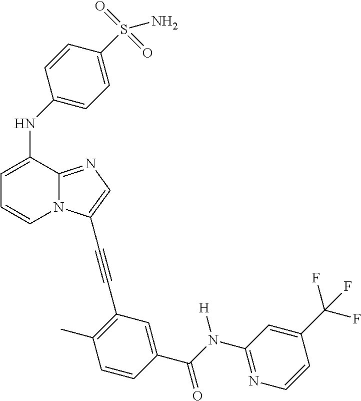 [SMILES: Cc1ccc(C(=O)Nc2cc(C(F)(F)F)ccn2)cc1C#Cc1cnc2c(Nc3ccc(S(N)(=O)=O)cc3)cccn12] | <1000 |
| 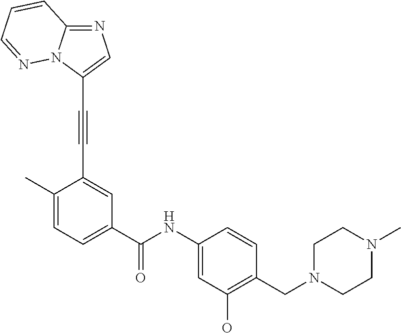 [SMILES: Cc1ccc(C(=O)Nc2ccc(CN3CCN(C)CC3)c(O)c2)cc1C#Cc1cnc2cccnn12] | <1000 |
| 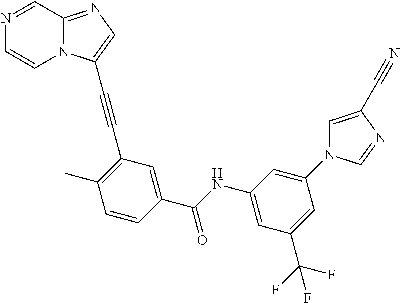 [SMILES: Cc1ccc(C(=O)Nc2cc(-n3cnc(C#N)c3)cc(C(F)(F)F)c2)cc1C#Cc1cnc2cnccn12] | <1000 |
| 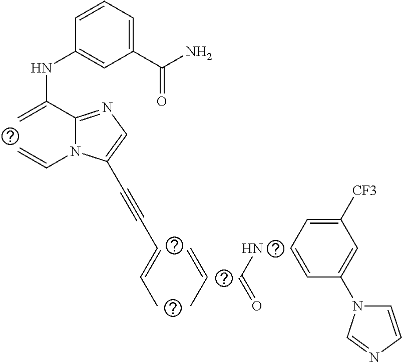 | <1000 |
| 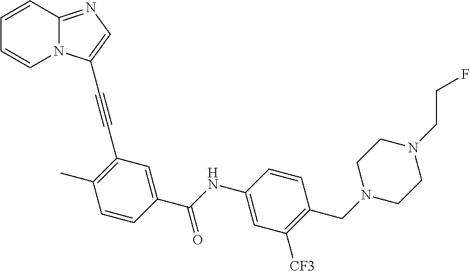 [SMILES: Cc1ccc(C(=O)Nc2ccc(CN3CCN(CCF)CC3)c(C(F)(F)F)c2)cc1C#Cc1cnc2ccccn12] | <1000 |
| 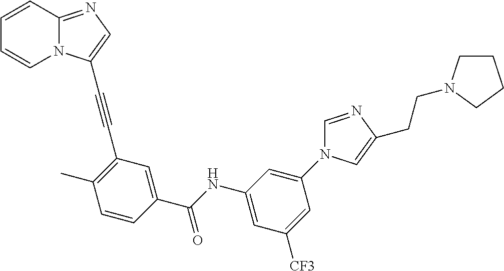 [SMILES: Cc1ccc(C(=O)Nc2cc(-n3cnc(CCN4CCCC4)c3)cc(C(F)(F)F)c2)cc1C#Cc1cnc2ccccn12] | <1000 |
| 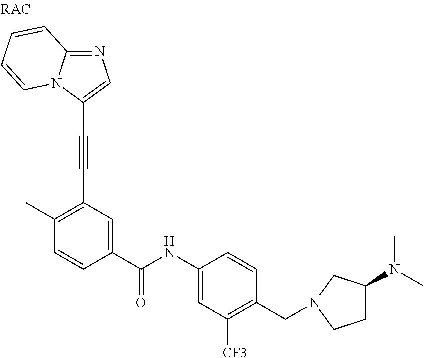 [SMILES: Cc1ccc(C(=O)Nc2ccc(CN3CC[C@H](N(C)C)C3)c(C(F)(F)F)c2)cc1C#Cc1cnc2ccccn12] | <1000 |
| 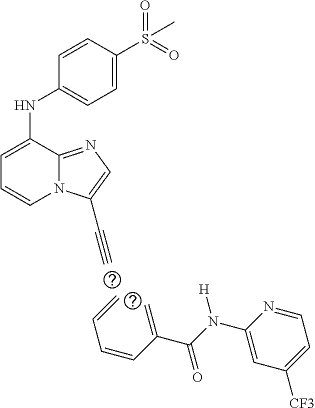 | <1000 |
| 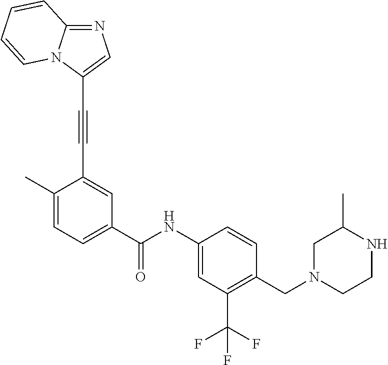 [SMILES: Cc1ccc(C(=O)Nc2ccc(CN3CCNC(C)C3)c(C(F)(F)F)c2)cc1C#Cc1cnc2ccccn12] | <1000 |
| 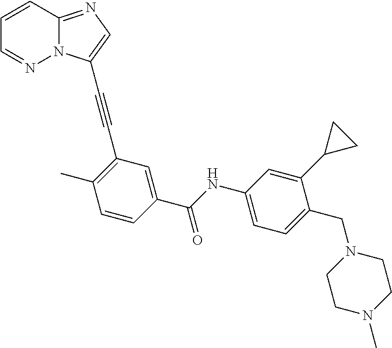 [SMILES: Cc1ccc(C(=O)Nc2ccc(CN3CCN(C)CC3)c(C3CC3)c2)cc1C#Cc1cnc2cccnn12] | <1000 |
| 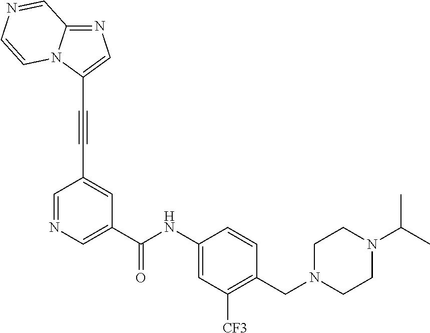 [SMILES: CC(C)N1CCN(Cc2ccc(NC(=O)c3cncc(C#Cc4cnc5cnccn45)c3)cc2C(F)(F)F)CC1] | <1000 |
| 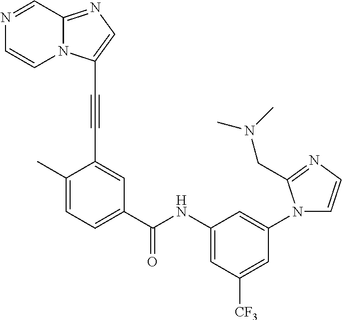 [SMILES: Cc1ccc(C(=O)Nc2cc(-n3ccnc3CN(C)C)cc(C(F)(F)F)c2)cc1C#Cc1cnc2cnccn12] | <1000 |
| 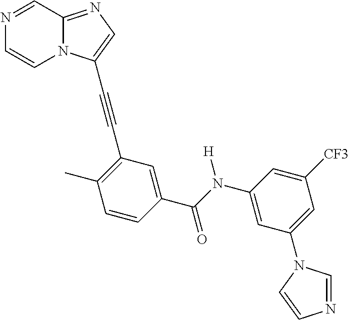 [SMILES: Cc1ccc(C(=O)Nc2cc(-n3ccnc3)cc(C(F)(F)F)c2)cc1C#Cc1cnc2cnccn12] | <1000 |
| 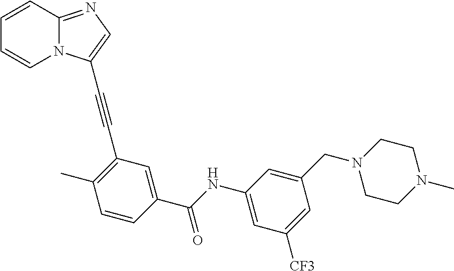 [SMILES: Cc1ccc(C(=O)Nc2cc(CN3CCN(C)CC3)cc(C(F)(F)F)c2)cc1C#Cc1cnc2ccccn12] | <1000 |
| 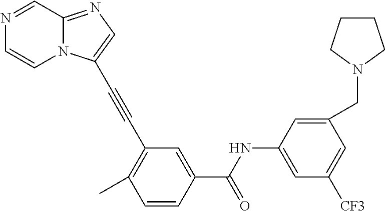 [SMILES: Cc1ccc(C(=O)Nc2cc(CN3CCCC3)cc(C(F)(F)F)c2)cc1C#Cc1cnc2cnccn12] | <1000 |
| 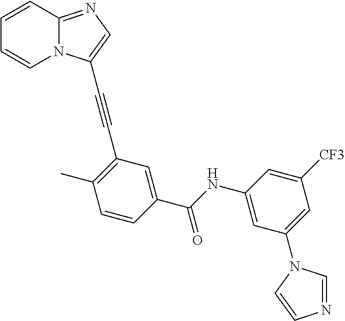 [SMILES: Cc1ccc(C(=O)Nc2cc(-n3ccnc3)cc(C(F)(F)F)c2)cc1C#Cc1cnc2ccccn12] | <1000 |
| 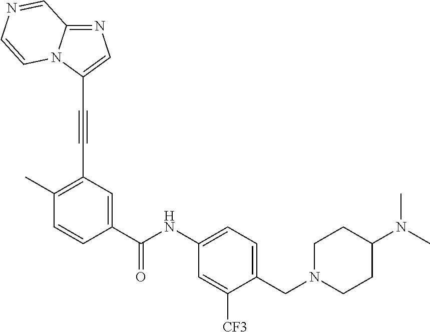 [SMILES: Cc1ccc(C(=O)Nc2ccc(CN3CCC(N(C)C)CC3)c(C(F)(F)F)c2)cc1C#Cc1cnc2cnccn12] | <1000 |
| 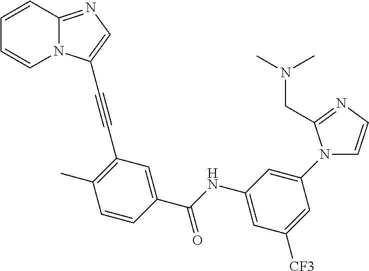 [SMILES: Cc1ccc(C(=O)Nc2cc(-n3ccnc3CN(C)C)cc(C(F)(F)F)c2)cc1C#Cc1cnc2ccccn12] | <1000 |
| 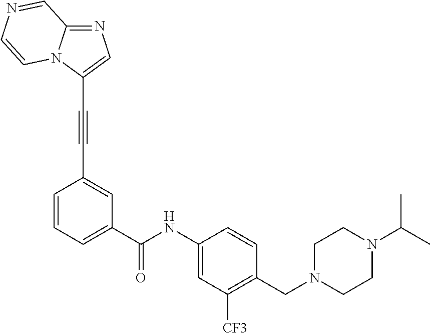 [SMILES: CC(C)N1CCN(Cc2ccc(NC(=O)c3cccc(C#Cc4cnc5cnccn45)c3)cc2C(F)(F)F)CC1] | <1000 |
| 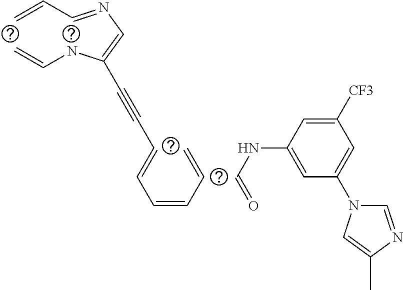 | <1000 |
| 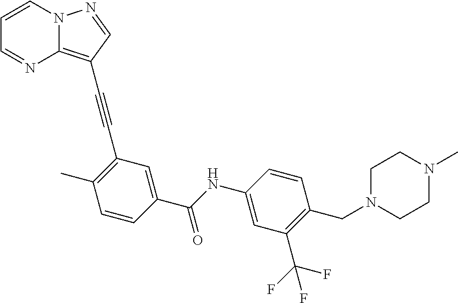 [SMILES: Cc1ccc(C(=O)Nc2ccc(CN3CCN(C)CC3)c(C(F)(F)F)c2)cc1C#Cc1cnn2cccnc12] | <1000 |
| 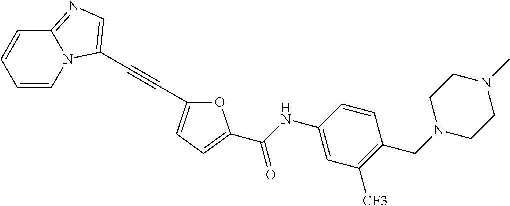 [SMILES: CN1CCN(Cc2ccc(NC(=O)c3ccc(C#Cc4cnc5ccccn45)o3)cc2C(F)(F)F)CC1] | <1000 |
| 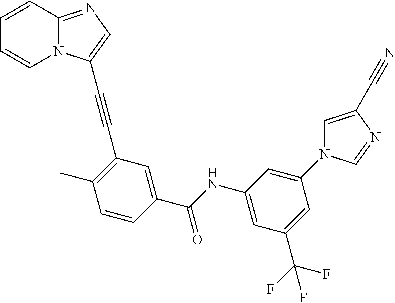 [SMILES: Cc1ccc(C(=O)Nc2cc(-n3cnc(C#N)c3)cc(C(F)(F)F)c2)cc1C#Cc1cnc2ccccn12] | <1000 |
| 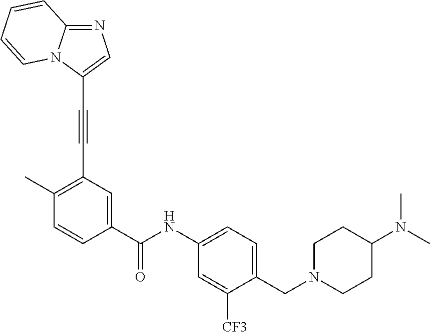 [SMILES: Cc1ccc(C(=O)Nc2ccc(CN3CCC(N(C)C)CC3)c(C(F)(F)F)c2)cc1C#Cc1cnc2ccccn12] | <1000 |
| 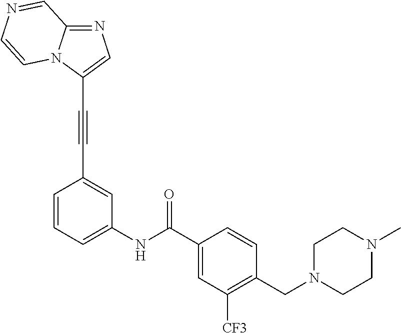 [SMILES: CN1CCN(Cc2ccc(C(=O)Nc3cccc(C#Cc4cnc5cnccn45)c3)cc2C(F)(F)F)CC1] | <1000 |
| 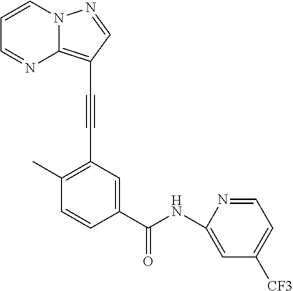 [SMILES: Cc1ccc(C(=O)Nc2cc(C(F)(F)F)ccn2)cc1C#Cc1cnn2cccnc12] | <1000 |
| 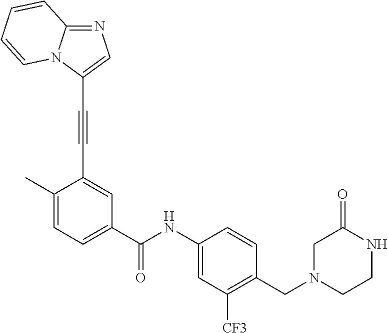 [SMILES: Cc1ccc(C(=O)Nc2ccc(CN3CCNC(=O)C3)c(C(F)(F)F)c2)cc1C#Cc1cnc2ccccn12] | <1000 |
| 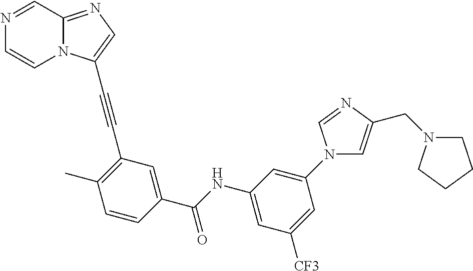 [SMILES: Cc1ccc(C(=O)Nc2cc(-n3cnc(CN4CCCC4)c3)cc(C(F)(F)F)c2)cc1C#Cc1cnc2cnccn12] | <1000 |
| 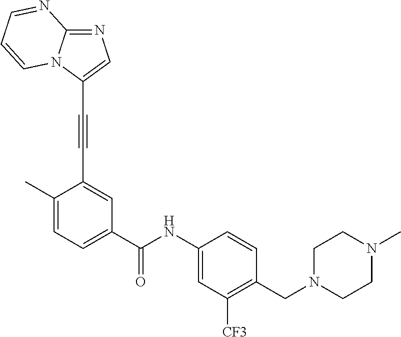 [SMILES: Cc1ccc(C(=O)Nc2ccc(CN3CCN(C)CC3)c(C(F)(F)F)c2)cc1C#Cc1cnc2ncccn12] | <1000 |
| 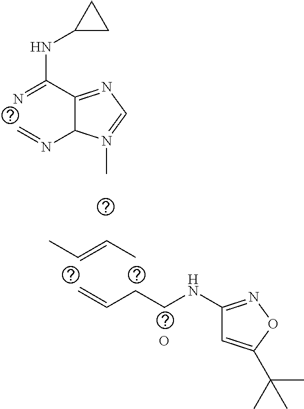 | <1000 |
| 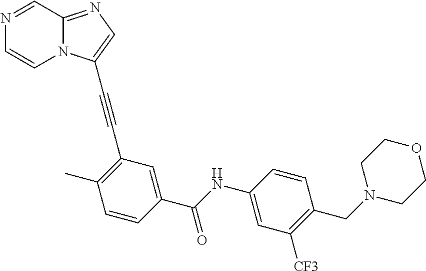 [SMILES: Cc1ccc(C(=O)Nc2ccc(CN3CCOCC3)c(C(F)(F)F)c2)cc1C#Cc1cnc2cnccn12] | <1000 |
| 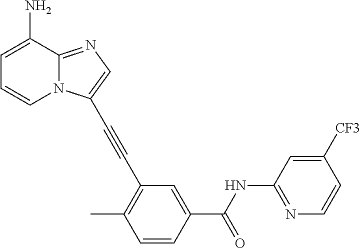 [SMILES: Cc1ccc(C(=O)Nc2cc(C(F)(F)F)ccn2)cc1C#Cc1cnc2c(N)cccn12] | <1000 |
| 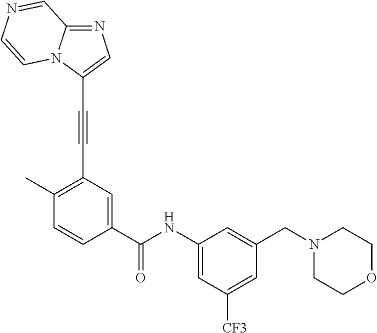 [SMILES: Cc1ccc(C(=O)Nc2cc(CN3CCOCC3)cc(C(F)(F)F)c2)cc1C#Cc1cnc2cnccn12] | <1000 |
| 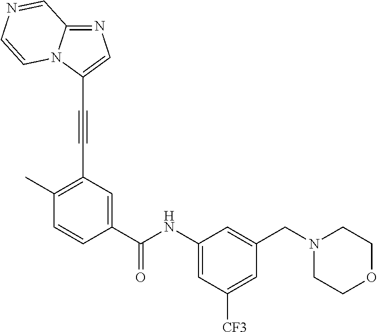 [SMILES: Cc1ccc(C(=O)Nc2cc(CN3CCOCC3)cc(C(F)(F)F)c2)cc1C#Cc1cnc2cnccn12] | <1000 |
| 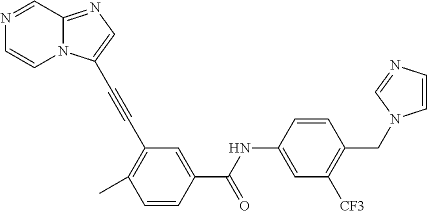 [SMILES: Cc1ccc(C(=O)Nc2ccc(Cn3ccnc3)c(C(F)(F)F)c2)cc1C#Cc1cnc2cnccn12] | <1000 |
| 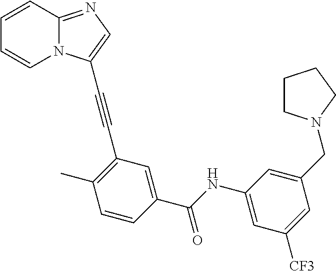 [SMILES: Cc1ccc(C(=O)Nc2cc(CN3CCCC3)cc(C(F)(F)F)c2)cc1C#Cc1cnc2ccccn12] | <1000 |
| 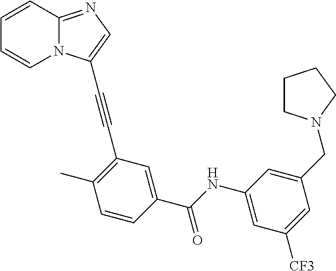 [SMILES: Cc1ccc(C(=O)Nc2cc(CN3CCCC3)cc(C(F)(F)F)c2)cc1C#Cc1cnc2ccccn12] | <1000 |
| 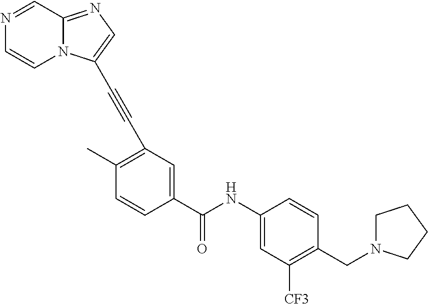 [SMILES: Cc1ccc(C(=O)Nc2ccc(CN3CCCC3)c(C(F)(F)F)c2)cc1C#Cc1cnc2cnccn12] | <1000 |
| 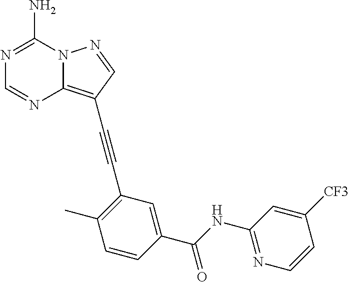 [SMILES: Cc1ccc(C(=O)Nc2cc(C(F)(F)F)ccn2)cc1C#Cc1cnn2c(N)ncnc12] | <1000 |
| 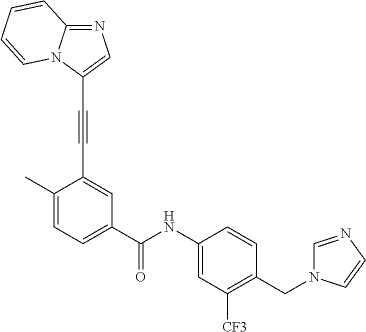 [SMILES: Cc1ccc(C(=O)Nc2ccc(Cn3ccnc3)c(C(F)(F)F)c2)cc1C#Cc1cnc2ccccn12] | <1000 |
| 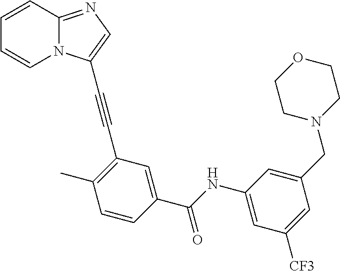 [SMILES: Cc1ccc(C(=O)Nc2cc(CN3CCOCC3)cc(C(F)(F)F)c2)cc1C#Cc1cnc2ccccn12] | <1000 |
| 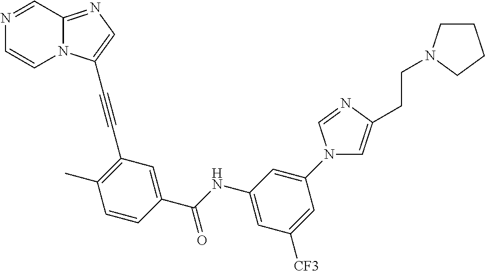 [SMILES: Cc1ccc(C(=O)Nc2cc(-n3cnc(CCN4CCCC4)c3)cc(C(F)(F)F)c2)cc1C#Cc1cnc2cnccn12] | <1000 |
| 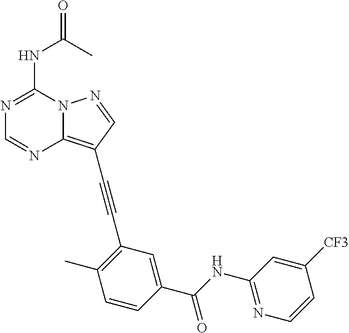 [SMILES: CC(=O)Nc1ncnc2c(C#Cc3cc(C(=O)Nc4cc(C(F)(F)F)ccn4)ccc3C)cnn12] | <1000 |
| 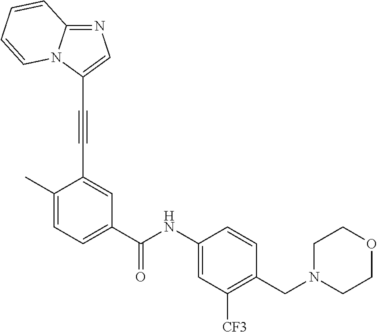 [SMILES: Cc1ccc(C(=O)Nc2ccc(CN3CCOCC3)c(C(F)(F)F)c2)cc1C#Cc1cnc2ccccn12] | <1000 |
| 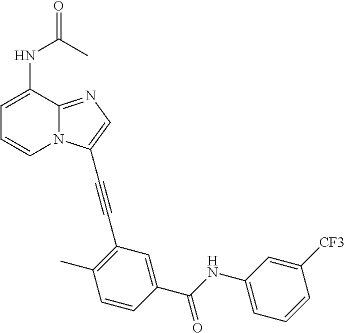 [SMILES: CC(=O)Nc1cccn2c(C#Cc3cc(C(=O)Nc4cccc(C(F)(F)F)c4)ccc3C)cnc12] | <1000 |
| 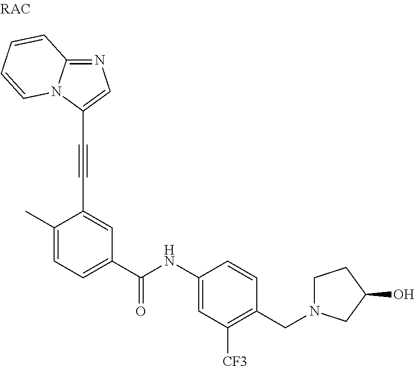 [SMILES: Cc1ccc(C(=O)Nc2ccc(CN3CC[C@@H](O)C3)c(C(F)(F)F)c2)cc1C#Cc1cnc2ccccn12] | <1000 |
| 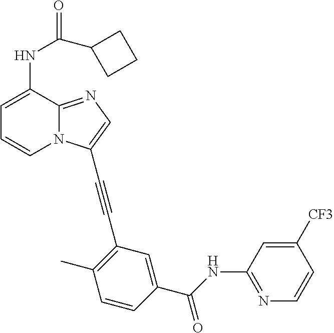 [SMILES: Cc1ccc(C(=O)Nc2cc(C(F)(F)F)ccn2)cc1C#Cc1cnc2c(NC(=O)C3CCC3)cccn12] | <1000 |
| 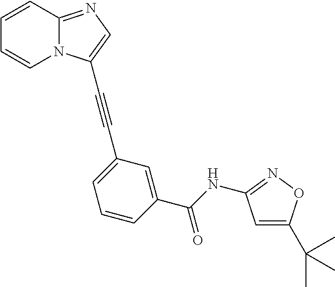 [SMILES: CC(C)(C)c1cc(NC(=O)c2cccc(C#Cc3cnc4ccccn34)c2)no1] | <1000 |

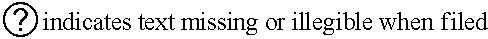

The compounds listed in the table below also showed inhibitory activity against various protein kinase of interest.

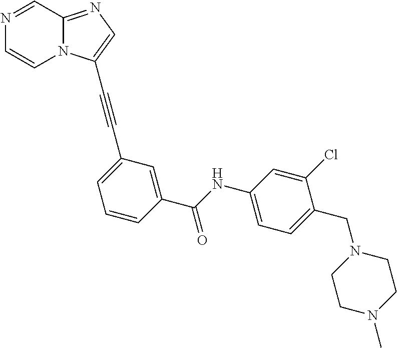 [SMILES: CN1CCN(Cc2ccc(NC(=O)c3cccc(C#Cc4cnc5cnccn45)c3)cc2Cl)CC1]

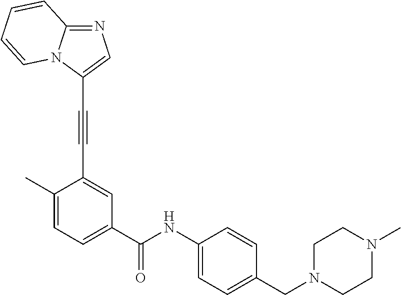 [SMILES: Cc1ccc(C(=O)Nc2ccc(CN3CCN(C)CC3)cc2)cc1C#Cc1cnc2ccccn12]

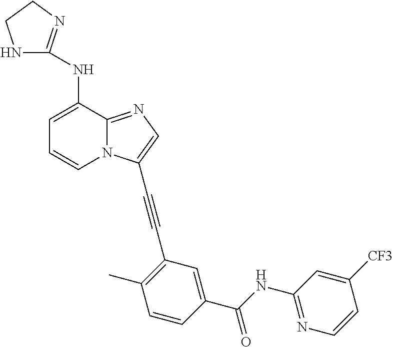 [SMILES: Cc1ccc(C(=O)Nc2cc(C(F)(F)F)ccn2)cc1C#Cc1cnc2c(NC3=NCCN3)cccn12]

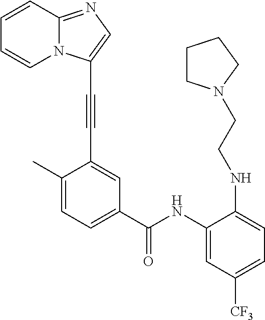 [SMILES: Cc1ccc(C(=O)Nc2cc(C(F)(F)F)ccc2NCCN2CCCC2)cc1C#Cc1cnc2ccccn12]

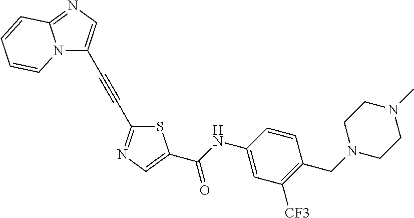 [SMILES: CN1CCN(Cc2ccc(NC(=O)c3cnc(C#Cc4cnc5ccccn45)s3)cc2C(F)(F)F)CC1]

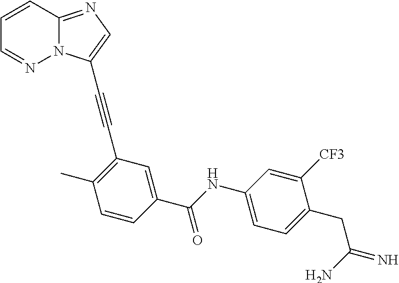 [SMILES: Cc1ccc(C(=O)Nc2ccc(CC(=N)N)c(C(F)(F)F)c2)cc1C#Cc1cnc2cccnn12]

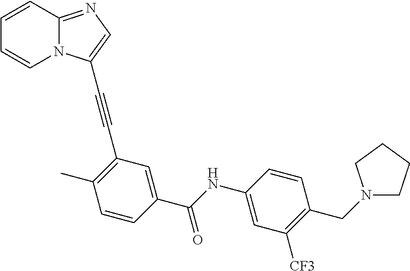 [SMILES: Cc1ccc(C(=O)Nc2ccc(CN3CCCC3)c(C(F)(F)F)c2)cc1C#Cc1cnc2ccccn12]

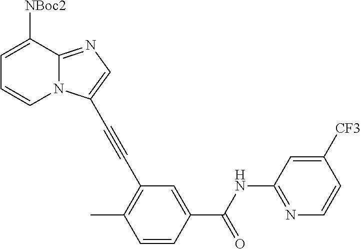

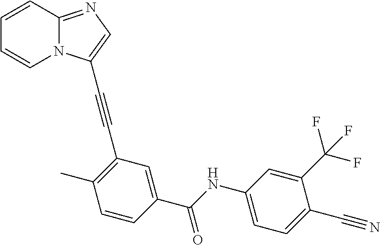 [SMILES: Cc1ccc(C(=O)Nc2ccc(C#N)c(C(F)(F)F)c2)cc1C#Cc1cnc2ccccn12]

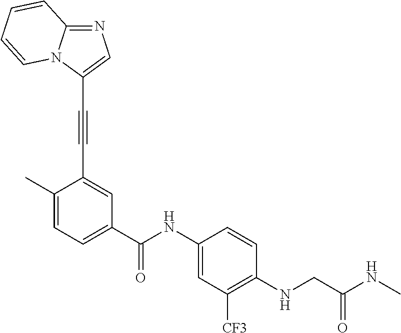 [SMILES: CNC(=O)CNc1ccc(NC(=O)c2ccc(C)c(C#Cc3cnc4ccccn34)c2)cc1C(F)(F)F]

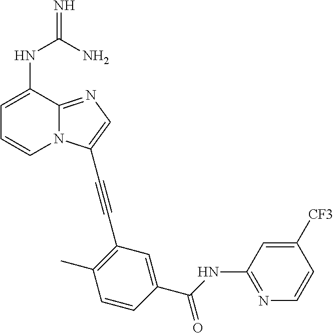 [SMILES: Cc1ccc(C(=O)Nc2cc(C(F)(F)F)ccn2)cc1C#Cc1cnc2c(NC(=N)N)cccn12]

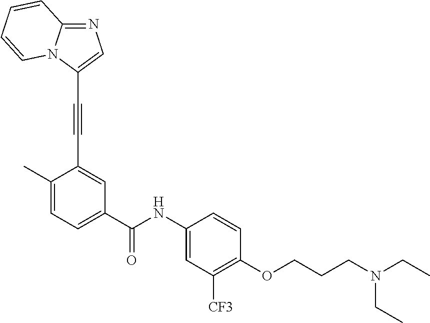 [SMILES: CCN(CC)CCCOc1ccc(NC(=O)c2ccc(C)c(C#Cc3cnc4ccccn34)c2)cc1C(F)(F)F]

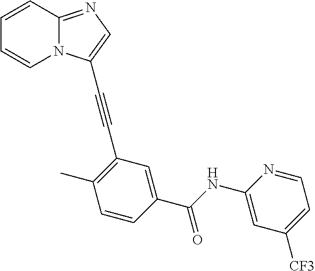 [SMILES: Cc1ccc(C(=O)Nc2cc(C(F)(F)F)ccn2)cc1C#Cc1cnc2ccccn12]

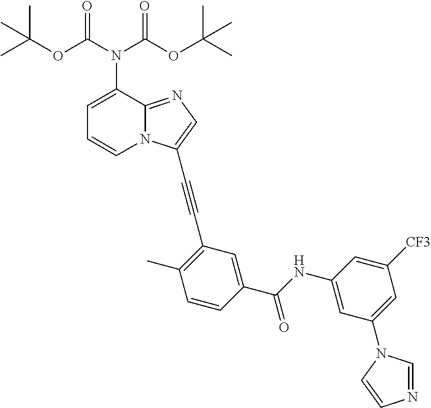 [SMILES: Cc1ccc(C(=O)Nc2cc(-n3ccnc3)cc(C(F)(F)F)c2)cc1C#Cc1cnc2c(N(C(=O)OC(C)(C)C)C(=O)OC(C)(C)C)cccn12]

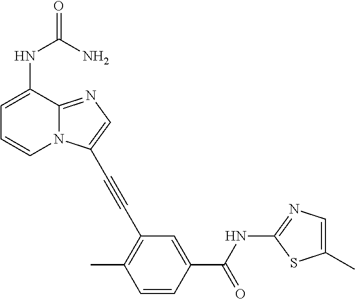 [SMILES: Cc1cnc(NC(=O)c2ccc(C)c(C#Cc3cnc4c(NC(N)=O)cccn34)c2)s1]

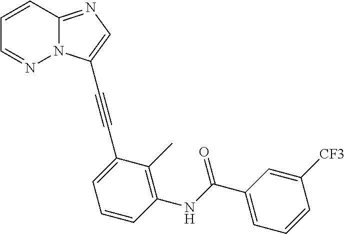 [SMILES: Cc1c(C#Cc2cnc3cccnn23)cccc1NC(=O)c1cccc(C(F)(F)F)c1]

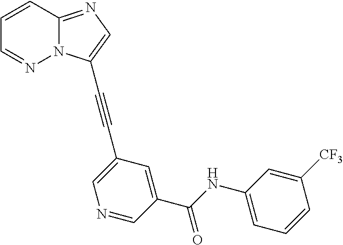 [SMILES: O=C(Nc1cccc(C(F)(F)F)c1)c1cncc(C#Cc2cnc3cccnn23)c1]

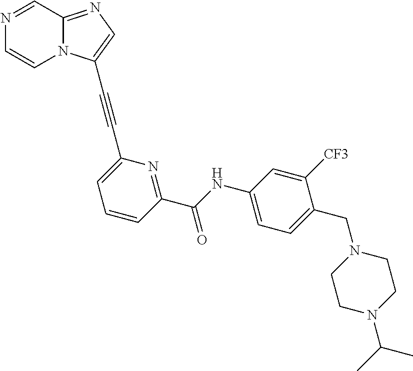 [SMILES: CC(C)N1CCN(Cc2ccc(NC(=O)c3cccc(C#Cc4cnc5cnccn45)n3)cc2C(F)(F)F)CC1]

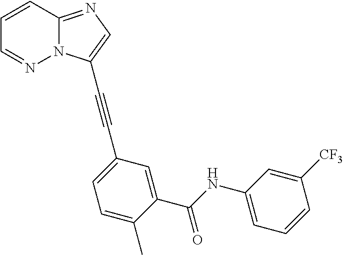 [SMILES: Cc1ccc(C#Cc2cnc3cccnn23)cc1C(=O)Nc1cccc(C(F)(F)F)c1]

Kinase Inhibition

More specifically, the compounds described herein are screened for kinase inhibition activity as follows. Kinases suitable for use in the following protocol include, but are not limited to those kinase identified in the definition of “targeted TKI” herein.

Kinases are expressed as either kinase domains or full length constructs fused to glutathione S-transferase (GST) or polyHistidine tagged fusion proteins in either E. coli or Baculovirus-High Five expression systems. They are purified to near homogeneity by affinity chromatography as previously described (Lehr et al., 1996; Gish et al., 1995). In some instances, kinases are co-expressed or mixed with purified or partially purified regulatory polypeptides prior to measurement of activity.

Kinase activity and inhibition can be measured by established protocols (see e.g., Braunwalder et al., 1996). In such cases, the transfer of 33P04 from ATP to the synthetic substrates poly(Glu, Tyr) 4:1 or poly(Arg, Ser) 3:1 attached to the bioactive surface of microtiter plates is taken as a measure of enzyme activity. After an incubation period, the amount of phosphate transferred is measured by first washing the plate with 0.5% phosphoric acid, adding liquid scintillant, and then counting in a liquid scintillation detector. The IC50 is determined by the concentration of compound that causes a 50% reduction in the amount of 33P incorporated onto the substrate bound to the plate.

In one method, the activated kinase is incubated with a biotinylated substrate peptide (containing tyr) with or without the presence of a compound of the invention. After the kinase assay incubation period, excess kinase inhibitor is added to kill the kinase reaction along with Europium-labeled anti-phosphotyrosine antibody (Eu-Ab) and Allophycocyanin-Streptavidin (SA-APC). The biotinylated substrate peptide (with or without phosphorylated Tyrosine) in solution binds to the SA-APC via Biotin-Avidin binding. The Eu-Ab binds only to substrate with phosphorylated tyrosine. When the solution is excited at 615 nm, there is an energy transfer from the Europium to the APC when they are in close proximity (i.e. attached to the same molecule of biotinylated and phosphorylated substrate peptide). The APC then fluoresces at a wavelength of 665 nm. Excitation and emission take place in a Wallac Victor² V plate reader where the plate is read fluorometrically and absorbances at 615 and 665 nm are recorded. These data are then processed by an Excel plate processor which calculates IC50s of test compounds by converting the fluorescence into amounts of phosphorylated substrate made and determining the concentration of test compound that would be required to inhibit the development of phosphorylated substrate by 50% (1050).

Other methods relying upon the transfer of phosphate to peptide or polypeptide substrate containing tyrosine, serine, threonine or histidine, alone, in combination with each other, or in combination with other amino acids, in solution or immobilized (i.e., solid phase) are also useful.

For example, transfer of phosphate to a peptide or polypeptide can also be detected using scintillation proximity, Fluorescence Polarization or homogeneous time-resolved fluorescence. Alternatively, kinase activity can be measured using antibody-based methods in which an antibody or polypeptide is used as a reagent to detect phosphorylated target polypeptide.

For additional background information on such assay methodologies, see e.g., Braunwalder et al., 1996, Anal. Biochem. 234(1):23; Cleaveland et al., 1990, Anal Biochem. 190(2):249 Gish et al. (1995). Protein Eng. 8(6):609 Kolb et al. (1998). Drug Discov. Toda V. 3:333 Lehr et al. (1996). Gene 169(2):27527-87 Seethala et al. (1998). Anal Biochem. 255(2):257 Wu et al. (2000).

Targeted TKI Activity:

The in vitro potency and selectivity of ponatinib was assessed in kinase assays with multiple recombinant kinase domains and peptide substrates in accordance with the procedures described in O'Hare, et al (cited herein). The resulting data are shown in the table immediately below:

### Kinase Inhibition Profile of Ponatinib for Native ABL, ABL^{T315I} and Selected Kinases

| Kinase | IC₅₀ (nM) |
|---|---|
| ABL | 0.37 |
| ABL^{T315I} | 2.0 |
| ABL^{Q252H} | 0.44 |
| ABL^{Y253F} | 0.30 |
| ABL^{M351T} | 0.30 |
| ABL^{H396P} | 0.34 |
| c SRC | 5.4 |
| LYN | 0.24 |
| c KIT | 12.5 |
| VEGFR2 | 1.5 |
| FGFR1 | 2.2 |
| PDGFRα | 1.1 |
| IR | >1000 |
| IGF 1R | >1000 |
| Aurora A | >1000 |
| CDK2/Cyclin E | >1000 |

It is evident from the data above that ponatinib demonstrated activity against various kinases, including Abl, PDGFR, src and c-kit in the low nanomolar range as determined by its IC50. More specifically, in addition to inhibiting Abl (IC₅₀: 0.37 nM) and Abl^{T315I}, (IC₅₀: 2.0 nM), ponatinib was determined to be a potent inhibitor of src (IC₅₀: 5.4 nM), PDGFR (IC₅₀: 1.1 nM) and c-kit (IC₅₀: 12.5 nM).

To determine the selectivity of ponatinib, it was subjected to a broader in vitro kinase assay panel in accordance with the procedures described in O'Hare, et al (cited herein). The resulting data are shown in the table immediately below:

### Ponatinib Kinase Panel Screening Data

TABLE S1
AP24534 Kinase Panel Screening Data

| IC₅₀ < 10 nM | | IC₅₀ < 50 nM | | IC₅₀ ≦ 250 nM | | IC₅₀ > 250 nM | |
|---|---|---|---|---|---|---|---|
| Kinase | IC₅₀ (nM) | Kinase | IC₅₀ (nM) | Kinase | IC₅₀ (nM) | Kinase | IC₅₀ (nM) |
| ABL | 0.37 | BMX | 47.2 | BRK | 50.6 | AKT2 | >1000 |
| ABL^{Q252H} | 0.44 | CSK | 12.7 | EGFR^{L858R} | 211 | ALK | >1000 |
| ABL^{Y253F} | 0.3 | DDR2 | 16.1 | EPHA1 | 143 | Aurora A | >1000 |
| ABL^{T3} [symbol] 5 [symbol] | 2 | EPHB4 | 10.2 | ERBB4 | 176 | Aurora B | 543 |
| ABL^{M351T} | 0.3 | FGFR3 | 18.2 | JAK2 | 169 | Aurora C | >1000 |
| ABL^{H396P} | 0.34 | FLT3 | 12.6 | JAK3 | 91.1 | AXL | >1000 |
| ARG | 0.76 | JAK1 | 32.2 | KIT^{V654A} | 77.8 | BTK | 849 |
| BLK | 6.1 | c-KIT | 12.5 | KIT^{O816V} | 152 | BTK^{E41K} | >1000 |
| EPHA2 | 2.1 | KIT^{D816H} | 16 | TYK2 | 177 | CDK2/CyclinE | >1000 |
| EPHA3 | 6.7 | PDGFRα^{O842V} | 15.6 | | | CTK | >1000 |
| EPHA4 | 1.1 | PYK2 | 35.1 | | | EGFR | >1000 |
| EPHA5 | 0.69 | TIE2 | 14.3 | | | EGFR^{L861Q} | 536 |
| EPHA7 | 8.5 | TRKA | 11.4 | | | EGFR^{T790V} | >1000 |
| EPHA8 | 2.5 | TRKB | 15.1 | | | ERBB2 | >1000 |
| EPHB1 | 1.2 | TRKC | 13.2 | | | FAK | >1000 |
| EPHB2 | 0.63 | | | | | FER | 560 |
| EPHB3 | 1.1 | | | | | FES | 768 |
| FGFR1 | 2.23 | | | | | FLT3^{O835Y} | 948 |
| FGFR1^{V561M} | 7.3 | | | | | IGF-1R | >1000 |
| FGFR2 | 1.6 | | | | | IR | >1000 |
| FGFR2^{N549H} | 0.45 | | | | | IRR | >1000 |
| FGFR4 | 7.7 | | | | | ITK | >1000 |
| FGR | 0.45 | | | | | c-MER | 406 |
| FMS | 8.6 | | | | | c-MET | >1000 |
| FRK | 1.3 | | | | | mTOR | >1000 |
| FYN | 0.36 | | | | | MUSK | 694 |
| HCK | 0.11 | | | | | PI3Kα | >1000 |
| KIT^{V550G} | 0.41 | | | | | PKA | 613 |
| LCK | 0.28 | | | | | PKC8 | >1000 |
| LYN | 0.24 | | | | | RON | >1000 |
| LYNB | 0.21 | | | | | ROS | >1000 |
| PDGFRα | 1.1 | | | | | SRC^{T341M} | >1000 |
| PDGFRα^{V561D} | 0.84 | | | | | SYK | >1000 |
| PDGFRα^{T674I} | 3 | | | | | TEC | >1000 |
| PDGFRβ | 7.7 | | | | | TYK1 | >1000 |
| RET | 0.16 | | | | | TYRO3 | >1000 |
| RET^{V804L} | 3.7 | | | | | ZAP70 | >1000 |
| RET^{V804M} | 1.4 | | | | | | |
| c-SRC | 5.4 | | | | | | |
| VEGFR1 | 3.7 | | | | | | |
| VEGFR2 | 1.5 | | | | | | |
| VEGFR3 | 2.3 | | | | | | |
| YES | 0.89 | | | | | | |

[symbol]

indicates data missing or illegible when filed

It is evident from the data in the table immediately above that ponatinib is not only a potent inhibitor of the kinases of interest for the methods disclosed herein (such as PDGFR, src, c-kit) but also a selective kinase inhibitor as apparent from its relative lack of activity against the kinases identified in the far right column (e.g., ALK, RON, etc.). This data coupled with the unexpected finding that ponatinib is able to cross the blood-brain barrier and accumulate there is fortuitous for the development of ponatinib for the treatment of neurodegenerative disease.

Cell-Based Assays

Certain compounds of this invention have also demonstrated cytotoxic or growth inhibitory effects on tumor and other cancer cell lines and thus may be useful in the treatment of cancer and other cell proliferative diseases. Compounds are assayed for anti-tumor activity using in vivo and in vitro assays which are well known to those skilled in the art. Generally, initial screens of compounds to identify candidate anti-cancer drugs are performed in cellular assays. Compounds identified as having anti-proliferative activity in such cell-based assays can then be subsequently assayed in whole organisms for anti-tumor activity and toxicity. Generally speaking, cell-based screens can be performed more rapidly and cost-effectively relative to assays that use whole organisms. For purposes of this invention, the terms “anti-tumor” and “anti-cancer” activity are used interchangeably.

An example of cell-based assay is shown as below. The cell lines used in the assay are Ba/F3, a murine pro-B cell line, which have been stably transfected with full-length wild-type Bcr-Abl or Bcr-Abl with various kinase domain point mutations (including T351I, Y253F, E255K, H396P, M351T etc.) constructs. Parental Ba/F3 cell line is used as control. These cell lines were obtained from Brian J. Druker (Howard Hughes Medical Institute, Oregon Health and Science University, Portland, Oreg., USA). Ba/F3 cell expressing Bcr-Abl or Bcr-Abl mutants were maintained in PRMI 1640 growth medium with 200 μM L-glutamine, 10% FCS, penicillin (200 U/ml), and streptomycin (200 μg/ml). Parental Ba/F3 cells were culture in the same medium supplemented with 10 ng/ml IL-3.

Parental Ba/F3 cells (supplemented with IL-3) or Ba/F3 cells expressing WT or mutant Bcr-Abl are plated in duplicate at 1×10⁴ cells/well in 96-well plates with the compounds in different concentrations in the media. The compounds are first dissolved and diluted in DMSO by preparation of 4-fold dilution; next equal volumes of compounds with DMSO are transferred to medium and then transferred to cell plates. The final compound concentrations start from 10 μM to 6 nM. DMSO at same percentage is used as control. After compound was incubated with cells for 3 days, the numbers of active cells are measured using CellTiter 96 AQueous One Solution Cell Proliferation assay kit following the kit instruction. Basically, the tetrazolium salts are added to the incubated cultured cells to allow enzymatic conversion to the detectable product by active cells. Cells are processed, and the optical density of the cells is determined to measure the amount of formazan derivatives. Mean+/− SD are generated from duplicated wells and reported as the percentage absorbance of control. IC50s are calculated in best-fit curves using Microsoft Excel-fit software.

Ponatinib Distribution into Brain Tissue

Ponatinib was formulated in 25 mM pH 2.75 citrate buffer and was orally administered (single dose) to rats at 5 mg/kg (and 5 mL/kg). At prescribed time points (1, 2, 4, 6, 24, and 48 hrs.), animals were anesthetized via carbon dioxide asphyxiation and whole blood samples were collected from the retro orbital sinus plexus. After blood collection, the rats were euthanized and the brains were removed by blunt dissection. The brain was rinsed, blotted dry and placed in a 50 mL conical centrifuge tube and frozen on dry ice. Once completely frozen, the brain was removed from the tube and weighed. The brain was stored at than −20° C. until analysis. Blood samples were stored at 4° C., if necessary, before being centrifuged at 15,000 rpm for 15 minutes. Plasma was removed and stored at −80° C. prior to analysis. The samples were analyzed by LC/MS/MS.

In the initial 2 hours after dosing, the plasma concentrations (ng/mL) were higher than brain concentrations (ng/gm), and Tₘₐₓ was same for brain and plasma at 4 hours. This indicates that the brain uptake has similar pharmacokinetics to the plasma pharmacokinetics and that ponatinib reaches brain tissue without much delay. The following additional data were obtained:

TABLE 1
Pharmacokinetics

| | | | Brain/Blood |
|---|---|---|---|
| | Blood | Brain | Ratio |
| Cmax (ng/mL) | 174.9 | 394.4 | 2.26 |
| Tmax (hr) | 4 | 4 | |
| Half-life (hr) | 6.65 | 10.32 | |
| AUC 0-inf | 2287 | 6384 | 2.79 |
| (hr · ng/mL) | | | |

These results indicate that in these experiments, exposure to ponatinib was 2.79 times greater in brain relative to blood on an area under the curve (AUC) basis and 2.26 times greater on a the basis of maximum concentration observed (Cmax). The observed elimination half-life was also longer in brain than in blood. These results are consistent with a previous study in which, after seven consecutive oral doses of ponatinib to female rats (10 mg/kg), the terminal 24-hr brain/plasma ratios were 2.27. Thus, therapy with ponatinib, or a salt thereof, results in ponatinib concentration in brain tissue that is more than twice that measured in the serum.

### Clinical Study

A phase 1 clinical trial was conducted to assess the safety of ponatinib hydrochloride in patients with refractory hematological cancers. This multi-center, sequential dose-escalation study was designed to determine the safety and tolerability of oral ponatinib, as well as its pharmacokinetics (behavior of the drug in patients) and its pharmacodynamics (the effects of the drug on patients' cells).

Subjects received the following dose levels: 2 mg (3 subjects), 4 mg (6 subjects), 8 mg (7 subjects), 15 mg (8 subjects), 30 mg (7 subjects), 45 mg (13 subjects), and 60 mg (13 subjects). In those subjects, 45 mg was identified as the maximum tolerated dose (MTD) for further investigation. Intra-patient dose escalation was permitted.

Preliminary safety data showed the following: for the 2 to 30 mg cohorts: no dose limiting toxicities (DLTs) were observed; for the 45 mg cohort: a reversible rash was seen with one patient; and for the 60 mg cohort: four patients developed reversible pancreatic related DLT (pancreatitis). The most common drug-related adverse events of any grade (AE) were thrombocytopenia (25%), anemia, lipase increase, nausea, and rash (12% each), and arthralgia, fatigue, and pancreatitis (11% each).

Pharmacokinetic data demonstrated that the half-life of ponatinib is 19-45 hours. At doses 30 mg, the half-life is 18 hours. The Cₘₐₓ on day 1 at the 30 mg dose was approximately 55 nM. After repeated dosing, 1.5 to 3-fold accumulation was observed in evaluable patients.

Pharmacokinetic data for patients receiving 60 mg of ponatinib daily is provided in Table 2.

TABLE 2
Profile of ponatinib orally administered at 60 mg (ng/mL)

| Period (hr) | Subject | 0 | 0.5 | 1 | 2 | 4 | 6 | 8 | 24 |
|---|---|---|---|---|---|---|---|---|---|
| 1 | A | BQL | 0.31 | 6.21 | 15.6 | 46.9 | 71.4 | 80.2 | 43.1 |
| | B | BQL | 2.92 | 8.35 | 12.2 | 31 | 29.5 | 22.7 | 17.9 |
| | C | BQL | 3.95 | 27 | 48.5 | 73 | 60.6 | 41.8 | 33.2 |
| | D | BQL | 11.6 | 27.8 | 56 | 151 | 151 | 135 | 51.6 |
| | E | BQL | 3.25 | 13.8 | 63.3 | 79.2 | 78.5 | 67 | 28.2 |
| | F | BQL | 22.1 | 39.7 | 56.6 | 65.6 | 56.4 | 46.9 | 22.3 |
| | G | BQL | BQL | 0.59 | 4.94 | 35.4 | 52.7 | 49.8 | 26.2 |
| | Mean | | 7.355 | 17.635 | 36.734 | 68.871 | 71.443 | 63.343 | 31.786 |
| 2 | A | 82 | 77.6 | 79.1 | 76.7 | 108 | 138 | 137 | 80.7 |
| | B | 30.1 | 36.8 | | 57.1 | 109 | 87.1 | 70.2 | 35.8 |
| | C | 61.5 | 67.4 | 78.5 | 94.8 | 94.7 | 85.3 | 72.1 | 47.6 |
| | D | 13.1 | 14.6 | 17.9 | 33.8 | 54.3 | 50.8 | 41 | 19.1 |
| | Mean | 46.675 | 49.1 | 58.5 | 65.6 | 91.5 | 90.3 | 80.075 | 45.8 |

The mean steady state trough level when dosing daily at 60 mg (the level at 24 hour post dosing following one 28-day cycle) is about 45 ng/mL, which corresponds to a circulating plasma concentration of about 90 nM. With doses of 30 mg or higher, trough levels surpassed a circulating plasma concentration of 40 nM (21 ng/mL).

Conclusions: This study showed ponatinib to be well tolerated and led to the selection of 45 mg daily dosing for leukemia patients in subsequent clinical study.

We have now found that ponatinib has a particularly favorable combination of properties permitting it to accumulate in brain tissue to pharmacologically useful levels, and in fact, at significantly higher levels than seen in serum, both in rodent and non-human primate studies (not shown). Those properties include the ability to cross the blood brain barrier to gain entry to the brain, relative freedom from removal from brain by virtue of being a poor substrate for the PGP efflux pump, and relative freedom from sequestration in protein-bound form, consistent with its favorable kinetics observed in protein binding experiments.

We have also found that the concentration of ponatinib in the brain tissue of the subjects receiving 30 mg of ponatinib can significantly exceed the concentration needed to increase the protective function of the gene product, parkin, in brain cells, in subjects with a neurodegenerative condition.

### OTHER EMBODIMENTS

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

1. A method for treating or preventing a neurodegenerative condition in a subject in need thereof comprising administering to the subject an effective amount of a targeted TKI, wherein the targeted TKI is a compound of Formula I: 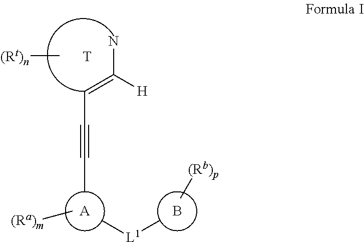
or a tautomer, or an individual isomer or a mixture of isomers thereof wherein:
Ring T is a 5-membered heteroaryl ring containing 1 or 2 nitrogens with the remaining ring atoms being carbon, substituted on at least two ring atoms with R^{t} groups, at least two of which being located on adjacent ring atoms, and, together with the atoms to which they are attached, forming a saturated, partially saturated or unsaturated 5- or 6-membered ring (Ring E), containing 0-3 heteroatoms selected from O, N, and S and being optionally substituted with 1-4 R^{e} groups;
Ring A is a 5- or 6-membered aryl or heteroaryl ring and is optionally substituted with 1-4 R^{a} groups;
Ring B is a 5- or 6-membered aryl or heteroaryl ring;
L¹ is selected from NR¹C(O), C(O)NR¹, NR¹C(O)O, NR¹C(O)NR¹, and OC(O)NR¹;
each occurrence of R^{a}, R^{b} and R^{t} is independently selected from the group consisting of halo, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;
R^{e}, at each occurrence, is independently selected from the group consisting of halo, ═O, —CN, —NO₂, —R⁴, —OR², —NR²R³, NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;
R¹, R² and R³ are independently selected from H, alkyl, alkenyl, alkynyl, cycloalkyl, cycloalkenyl, cycloalkynyl, aryl, heterocyclic and heteroaryl;
alternatively, R² and R³, taken together with the atom to which they are attached, form a 5- or 6-membered saturated, partially saturated or unsaturated ring, which can be optionally substituted and which contains 0-2 heteroatoms selected from N, O and S(O)ᵣ;
each occurrence of R⁴ is independently selected from alkyl, alkenyl, alkynyl, cycloalkyl, cycloalkenyl, cycloalkynyl, aryl, heterocyclic and heteroaryl;
each of the alkyl, alkenyl, alkynyl, cycloalkyl, cycloalkenyl, cycloalkynyl, aryl, heterocyclic and heteroaryl moieties is optionally substituted;
m is 0, 1, 2, 3 or 4;
n is 2 or 3;
p is 0, 1, 2, 3, 4 or 5; and,
r is 0, 1 or 2;
or a pharmaceutically acceptable salt, solvate or hydrate thereof.

2. A method according to claim 1, wherein in the compound of Formula I, Ring T is: 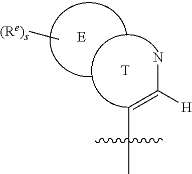
wherein Ring E is a 5- or 6-membered unsaturated ring comprising 0-3 heteroatoms selected from O, N, and S, and s is 0, 1, 2, 3 or 4.

3. A method according to claim 1, wherein in the compound of Formula I, Ring T is a bicyclic heteroaryl ring selected from: 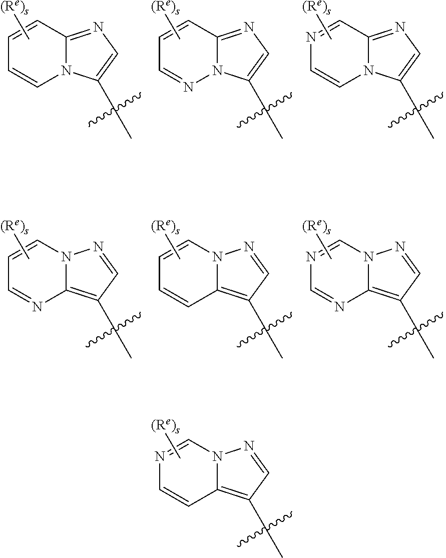
and s is 0, 1, 2, 3 or 4.

4. A method according to claim 1, wherein the targeted TKI is a compound of Formula II: 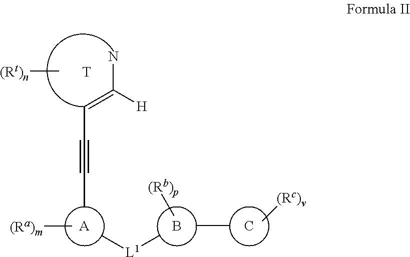
wherein:
Ring C is a 5- or 6-membered heterocyclic or heteroaryl ring, comprising carbon atoms and 1-3 heteroatoms independently selected from O, N and S(O)ᵣ;
R^{c}, at each occurrence, is independently selected from halo, ═O, —CN, —NO₂, —R⁴, —OR², —NR²R³, —C(O)YR², —OC(O)YR², —NR²C(O)YR², —Si(R²)₃, —SC(O)YR², —NR²C(═S)YR², —OC(═S)YR², —C(═S)YR², —YC(═NR³)YR², —YP(═O)(YR⁴)(YR⁴), —NR²SO₂R², —S(O)ᵣR², —SO₂NR²R³ and —NR²SO₂NR²R³, wherein each Y is independently a bond, —O—, —S— or —NR3-; and,
v is 0, 1, 2, 3, 4 or 5.

5. A method according to claim 4, wherein Ring T is: 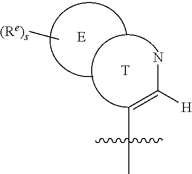
wherein Ring E is a 5- or 6-membered unsaturated ring comprising 0-3 heteroatoms selected from O, N, and S, and s is 0, 1, 2, 3 or 4.

6. A method according to claim 5, wherein Rings A and B are aryl.

7. A method according to claim 5, wherein Ring C is imidazolyl.

8. A method according to claim 7, wherein the targeted TKI is a compound selected from Formulae IIa, IIb, or IIc:

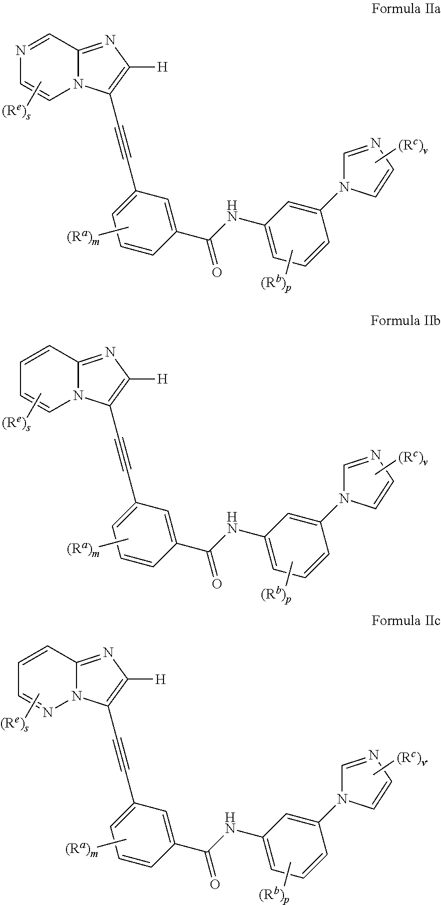

9. A method according to claim 8, wherein s is 0; m, p and v are 1; R^{a} and R^{c} are methyl; and R^{b} is CF₃.

10. A method according to claim 1, wherein the targeted TKI is a compound of Formula III: 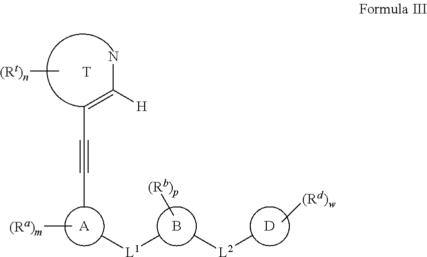
wherein:
Ring D represents a 5-, 6-heterocyclic or heteroaryl ring comprising carbon atoms and 1-3 heteroatoms independently selected from O, N and S(O)ᵣ;
L² is (CH₂)_{z}, O(CH₂)ₓ, NR³(CH₂)ₓ, S(CH₂)ₓ or (CH₂)ₓNR³C(O)(CH₂)ₓ in either direction;
R^{d}, at each occurrence, is selected from the group consisting of H, halo, ═O, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR3-;
R¹, R² and R³ are independently selected from H, alkyl, alkenyl, alkynyl, cycloalkyl, cycloalkenyl, cycloalkynyl, aryl, heterocyclic and heteroaryl;
alternatively, R² and R³, taken together with the atom to which they are attached, form a 5- or 6-membered saturated, partially saturated or unsaturated ring, which can be optionally substituted and which contains 0-2 heteroatoms selected from N, O and S(O)ᵣ;
each occurrence of R⁴ is independently selected from alkyl, alkenyl, alkynyl, cycloalkyl, cycloalkenyl, cycloalkynyl, aryl, heterocyclic and heteroaryl;
each of the alkyl, alkenyl, alkynyl, cycloalkyl, cycloalkenyl, cycloalkynyl, aryl, heterocyclic and heteroaryl moieties is optionally substituted;
p is 0, 1, 2, 3 or 4;
w is 0, 1, 2, 3, 4 or 5;
x is 0, 1, 2 or 3; and,
z is 1, 2, 3 or 4.

11. A method according to claim 10, wherein Ring T has the following structure: 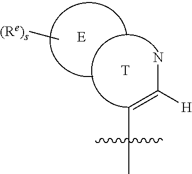
wherein Ring E is a 5- or 6-membered unsaturated ring comprising 0-3 heteroatoms selected from O, N, and S, and s is 0, 1, 2, 3 or 4.

12. A method according to claim 11, wherein Rings A and B are aryl.

13. A method according to claim 11, wherein Ring T is a bicyclic heteroaryl ring selected from: 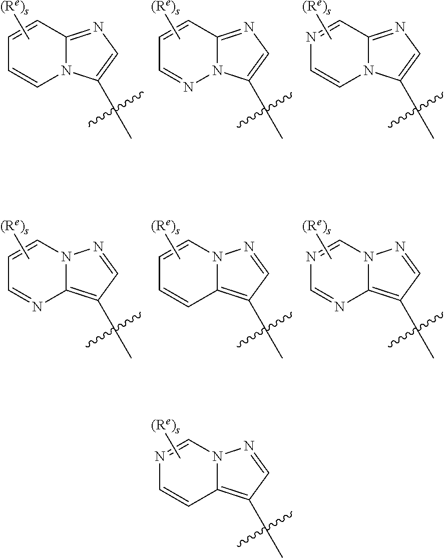
and s is 0, 1, 2, 3 or 4.

14. A method according to claim 13, wherein Ring D is piperazinyl and L² is CH₂.

15. A method according to claim 14 wherein the targeted TKI is a compound selected from Formulae IIIa, IIIb, and IIIc:

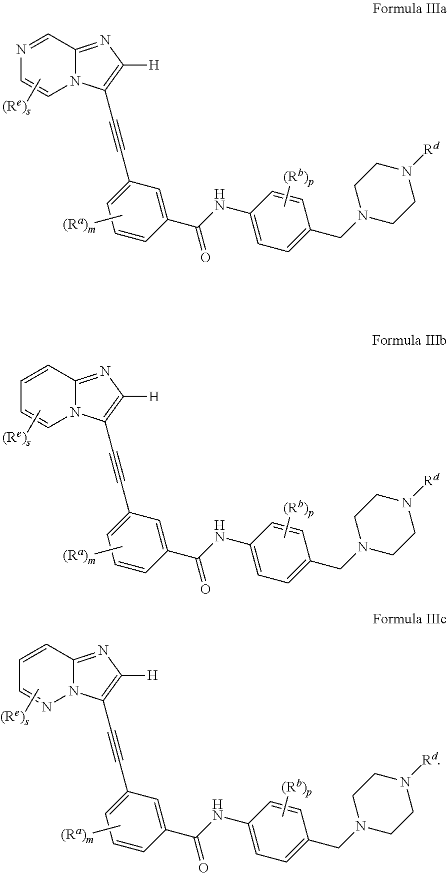

16. A method according to claim 15 wherein s is 0, m is 1, p is 1, R^{a} is methyl, R^{b} is CF₃, and R^{d} is methyl or —CH₂CH₂OH.

17. A method according to claim 1, wherein the targeted TKI is a compound selected from the group consisting of:
N-(3-(1H-imidazol-1-yl)-5-(trifluoromethyl)phenyl)-3-(imidazo[1,2-a]pyrazin-3-ylethynyl)-4-methylbenzamide;
3-(Imidazo[1,2-a]pyrazin-3-ylethynyl)-4-methyl-N-(4-((4-methylpiperazin-1-yl)methyl)-3-(trifluoromethyl)phenyl)benzamide;
N-(3-(2-((dimethylamino)methyl)-1H-imidazol-1-yl)-5-(trifluoromethyl)phenyl)-3-(imidazo[1,2-a]pyrazin-3-ylethynyl)-4-methylbenzamide;
3-(Imidazo[1,2-a]pyridin-3-ylethynyl)-4-methyl-N-(3-(4-methyl-1H-imidazol-1-yl)-5-(trifluoromethyl)phenyl)benzamide;
N-(3-(1H-imidazol-1-yl)-5-(trifluoromethyl)phenyl)-3-(imidazo[1,2-a]pyridin-3-ylethynyl)-4-methylbenzamide;
3-(Imidazo[1,2-a]pyridin-3-ylethynyl)-4-methyl-N-(4-(trifluoromethyl)pyridin-2-yl)benzamide;
N-(5-tert-butylisoxazol-3-yl)-3-(imidazo[1,2-a]pyridin-3-ylethynyl)-4-methylbenzamide;
3-(Imidazo[1,2-a]pyridin-3-ylethynyl)-4-methyl-N-(4-((4-methylpiperazin-1-yl)methyl)-3-(trifluoromethyl)phenyl)benzamide;
N-(3-(2-((dimethylamino)methyl)-1H-imidazol-1-yl)-5-(trifluoromethyl)phenyl)-3-(imidazo[1,2-a]pyridin-3-ylethynyl)-4-methylbenzamide;
3-((8-Acetamidoimidazo[1,2-a]pyridin-3-yl)ethynyl)-4-methyl-N-(4-(trifluoromethyl)pyridin-2-yl)benzamide;
N-(3-(1H-imidazol-1-yl)-5-(trifluoromethyl)phenyl)-3-((8-acetamidoimidazo[1,2-a]pyridin-3-yl)ethynyl)-4-methylbenzamide;
4-Methyl-3-((8-(4-(methylsulfonyl)phenylamino)imidazo[1,2-a]pyridin-3-yl)ethynyl)-N-(4-(trifluoromethyl)pyridin-2-yl)benzamide;
4-methyl-3-((8-(4-sulfamoylphenylamino)imidazo[1,2-a]pyridin-3-yl)ethynyl)-N-(4-(trifluoromethyl)pyridin-2-yl)benzamide;
(R)—N-(4-((3-(Dimethylamino)pyrrolidin-1-yl)methyl)-3-(trifluoromethyl)phenyl)-3-(imidazo[1,2-b]pyridazin-3-ylethynyl)-4-methylbenzamide;
N-(3-(Imidazo[1,2-b]pyridazin-3-ylethynyl)-4-methylphenyl)-4-((4-methylpiperazin-1-yl)methyl)-3-(trifluoromethyl)benzamide;
3-(Imidazo[1,2-b]pyridazin-3-ylethynyl)-4-methyl-N-(4-((4-methylpiperazin-1-yl)methyl)-3-(trifluoromethyl)phenyl)benzamide;
N-(3-Chloro-4-(methylpiperazin-1-yl)methyl)phenyl)-3-(imidazo[1,2-b]pyridazin-3-ylethynyl)-4-methylbenzamide;
N-(3-Cyclopropyl-4-((4-methylpiperazin-1-yl)methyl)phenyl)-3-(imidazo[1,2-b]pyridazin-3-ylethynyl)-4-methylbenzamide;
3-(Imidazo[1,2-b]pyridazin-3-ylethynyl)-N-(4-((4-methylpiperazin-1-yl)methyl)-3-(trifluoromethyl)phenyl)benzamide;
N-(4-((4-(2-Hydroxyethyl)piperazin-1-yl)methyl)-3-(trifluoromethyl)phenyl)-3-(imidazo[1,2-b]pyridazin-3-ylethynyl)-4-methylbenzamide; and
3-(Imidazo[1,2-b]pyridazin-3-ylethynyl)-4-methyl-N-(4-(piperazin-1-ylmethyl)-3-(trifluoromethyl)phenyl)benzamide,
or a pharmaceutically acceptable salt thereof.

18. A method according to claim 17, wherein the targeted TKI is 3-(Imidazo[1,2-b]pyridazin-3-ylethynyl)-4-methyl-N-(4-((4-methylpiperazin-1-yl)methyl)-3-(trifluoromethyl)phenyl)benzamide or a pharmaceutically acceptable salt thereof.

19. A method according to claim 1, wherein the neurodegenerative condition is associated with mitochondrial dysfunction and is selected from the group consisting of Friedrich's ataxia, amyotrophic lateral sclerosis, mitochondrial myopathy, encephalopathy, lactacidosis, stroke (MELAS), myoclonic epilepsy with ragged red fibers (MERFF), epilepsy, and Huntington's Disease.

20. A method according to claim 1, wherein the neurodegenerative condition is a tau pathology and is selected from the group consisting of Alzheimer's disease, progressive supranuclear palsy, Pick's disease, corticobasal degeneration, and fronto-temporal dementia linked to chromosome 17 with parkinsonism.

21. A method accordingly to claim 1, wherein the neurodegenerative condition is multiple sclerosis.

22. A method according to claim 1, wherein the targeted TKI, or a pharmaceutically acceptable salt thereof, is administered orally or intravenously.

23. A method according to claim 1, wherein the effective amount of the targeted TKI, or a pharmaceutically acceptable salt thereof, is about 5 mg to about 80 mg.

24. A method according to claim 1, wherein the targeted TKI, or a pharmaceutically acceptable salt thereof, is administered to the subject more than one day a week or on average 4 to 7 times every 7 day period.

25. A method according to claim 24, wherein the targeted TKI, or a pharmaceutically acceptable salt thereof, is administered to the subject daily.

26. A method according to claim 25, wherein an average daily dose of 5±2 mg, 8±2 mg, 12±3 mg, 15±3 mg, 20±4 mg, 25±5 mg, 30±6 mg, 40±8 mg, 45±9 mg, 50±10 mg, or 55±11 mg of the targeted TKI, or a pharmaceutically acceptable salt thereof, is administered to the subject.

27. A method for treating or preventing Alzheimer's disease in a subject in need thereof comprising administering to the subject a targeted TKI in an amount sufficient to inhibit γ-secretase in the brain of the subject, wherein the targeted TKI is a compound of Formula I: 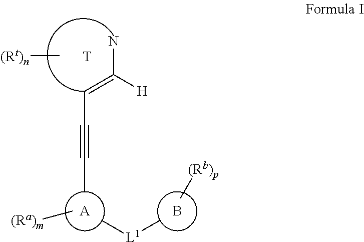
or a tautomer, or an individual isomer or a mixture of isomers thereof wherein:
Ring T is a 5-membered heteroaryl ring containing 1 or 2 nitrogens with the remaining ring atoms being carbon, substituted on at least two ring atoms with R^{t} groups, at least two of which being located on adjacent ring atoms, and, together with the atoms to which they are attached, forming a saturated, partially saturated or unsaturated 5- or 6-membered ring (Ring E), containing 0-3 heteroatoms selected from O, N, and S and being optionally substituted with 1-4 R^{e} groups;
Ring A is a 5- or 6-membered aryl or heteroaryl ring and is optionally substituted with 1-4 R^{a} groups;
Ring B is a 5- or 6-membered aryl or heteroaryl ring;
L¹ is selected from NR¹C(O), C(O)NR¹, NR¹C(O)O, NR¹C(O)NR¹, and OC(O)NR¹;
each occurrence of R^{a}, R^{b} and R^{t} is independently selected from the group consisting of halo, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;
R^{e}, at each occurrence, is independently selected from the group consisting of halo, ═O, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, NR²SO₂R², —S(O)ᵣR², —SO₂NR²R³ and —NR²SO₂NR²R³, wherein each Y is independently a bond, —O—, —S— or —NR³—;
R¹, R² and R³ are independently selected from H, alkyl, alkenyl, alkynyl, cycloalkyl, cycloalkenyl, cycloalkynyl, aryl, heterocyclic and heteroaryl;
alternatively, R² and R³, taken together with the atom to which they are attached, form a 5- or 6-membered saturated, partially saturated or unsaturated ring, which can be optionally substituted and which contains 0-2 heteroatoms selected from N, O and S(O)ᵣ;
each occurrence of R⁴ is independently selected from alkyl, alkenyl, alkynyl, cycloalkyl, cycloalkenyl, cycloalkynyl, aryl, heterocyclic and heteroaryl;
each of the alkyl, alkenyl, alkynyl, cycloalkyl, cycloalkenyl, cycloalkynyl, aryl, heterocyclic and heteroaryl moieties is optionally substituted;
m is 0, 1, 2, 3 or 4;
n is 2 or 3;
p is 0, 1, 2, 3, 4 or 5; and,
r is 0, 1 or 2;
or a pharmaceutically acceptable salt, solvate or hydrate thereof.

28. A pharmaceutical composition for treating or preventing a neurodegenerative condition in a subject in need thereof comprising an effective amount of a targeted TKI, wherein the targeted TKI is a compound of Formula I: 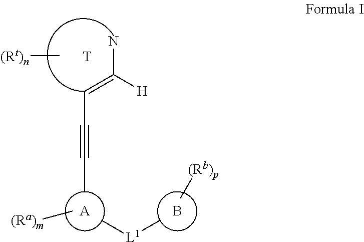
or a tautomer, or an individual isomer or a mixture of isomers thereof wherein:
Ring T is a 5-membered heteroaryl ring containing 1 or 2 nitrogens with the remaining ring atoms being carbon, substituted on at least two ring atoms with R^{t} groups, at least two of which being located on adjacent ring atoms, and, together with the atoms to which they are attached, forming a saturated, partially saturated or unsaturated 5- or 6-membered ring (Ring E), containing 0-3 heteroatoms selected from O, N, and S and being optionally substituted with 1-4 R^{e} groups;
Ring A is a 5- or 6-membered aryl or heteroaryl ring and is optionally substituted with 1-4 R^{a} groups;
Ring B is a 5- or 6-membered aryl or heteroaryl ring;
L¹ is selected from NR¹C(O), C(O)NR¹, NR¹C(O)O, NR¹C(O)NR¹, and OC(O)NR¹;
each occurrence of R^{a}, R^{b} and R^{t} is independently selected from the group consisting of halo, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;
R^{e}, at each occurrence, is independently selected from the group consisting of halo, ═O, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;
R¹, R² and R³ are independently selected from H, alkyl, alkenyl, alkynyl, cycloalkyl, cycloalkenyl, cycloalkynyl, aryl, heterocyclic and heteroaryl;
alternatively, R² and R³, taken together with the atom to which they are attached, form a 5- or 6-membered saturated, partially saturated or unsaturated ring, which can be optionally substituted and which contains 0-2 heteroatoms selected from N, O and S(O)ᵣ;
each occurrence of R⁴ is independently selected from alkyl, alkenyl, alkynyl, cycloalkyl, cycloalkenyl, cycloalkynyl, aryl, heterocyclic and heteroaryl;
each of the alkyl, alkenyl, alkynyl, cycloalkyl, cycloalkenyl, cycloalkynyl, aryl, heterocyclic and heteroaryl moieties is optionally substituted;
m is 0, 1, 2, 3 or 4;
n is 2 or 3;
p is 0, 1, 2, 3, 4 or 5; and,
r is 0, 1 or 2;
or a pharmaceutically acceptable salt, solvate or hydrate thereof; and
a pharmaceutically acceptable carrier.
